(19) United States
(12) Patent Application Publication
(10) Pub. No.: US 2016/0375041 A1
(43) Pub. Date: Dec. 29, 2016

(54) INHIBITION OF ISOPRENOID BIOSYNTHETIC PATHWAYS TO TREAT AUTOIMMUNE DISORDERS

(71) Applicant: David A. TABACZYNSKI, Cohasset, MA (US)

(72) Inventor: David A. TABACZYNSKI, Cohasset, MA (US)

(21) Appl. No.: 15/039,888

(22) PCT Filed: Dec. 1, 2014
(86) PCT No.: PCT/US2014/067911

§ 371 Date: May 27, 2016

Related U.S. Application Data
(60) Provisional application No. 61/910,598, filed on Dec. 2, 2013.
(60) Provisional application No. 62/019,516, filed on Jul. 1, 2014.

(51) Int. Cl. A61K 31/662; A61K 31/663; A61K 31/56; A61K 31/65; A61K 31/18; A61K 31/16; A61K 31/519; A61K 45/06

(52) U.S. Cl. CPC A61K 31/662; A61K 31/519; A61K 31/663; A61K 45/06; A61K 31/65; A61K 31/18; A61K 31/16; A61K 31/56

(57) ABSTRACT

The invention provides methods and pharmaceutical compositions that can treat autoimmune disease by reducing the production of pyrophosphate intermediates produced during the biosynthesis of isoprenoids. The pyrophosphate compounds being inhibited are normally produced through the mevalonate and non-mevalonate pathways of the host vertebrate organisms and their symbiotic and pathogenic microorganisms. The methods involve administering to a patient an inhibitor of the mevalonate-dependent pathway, an inhibitor of the non-mevalonate pathway, or combination of such inhibitors.

### CROSS REFERENCE TO RELATED APPLICATIONS

This application claims the benefit of and priority to U.S. Provisional Patent Application Ser. No. 61/910,598, filed Dec. 2, 2013, and U.S. Provisional Patent Application Ser. No. 62/019,516, filed Jul. 1, 2014, the contents of each of which are hereby incorporated by reference.

### FIELD OF THE INVENTION

This disclosure relates to pharmaceutical compositions that can reduce the production of pyrophosphate intermediates produced during the biosynthesis of isoprenoids to treat a number of autoimmune diseases.

### BACKGROUND

Pyrophosphates produced as intermediates during isoprenoid biosynthesis are key regulatory and stimulatory molecules in the inflammatory process associated with autoimmunity. In particular, isopentenyl pyrophosphate (IPP) and (E)-4-Hydroxy-3-methyl-but-2-enyl pyrophosphate (HMBPP) interact with particular γδ T Cell Receptors (TCR) known to drive inflammation that is related to autoimmunity.

The main sources of these two key stimulatory pyrophosphates are as intermediates from the isoprenoid biosynthetic pathways. In particular, these intermediates are the MVA pathway branch (also known as the mevalonate or HMG-CoA Reductase pathway) and MEP pathway branch (also known as the non-mevalonate, 2-C-methyl-D-erythritol 4-phosphate, 1-deoxy-D-xylulose 5-phosphate, or DOXP pathway) of isoprenoid biosynthesis. IPP is made in both the MVA and MEP pathways, whereas HMBPP is made exclusively in the MEP pathway.

Presence of IPP and HMBPP results in stimulation, differentiation, and proliferation of γδ (gamma delta) lymphoid cells. These pyrophosphates act as non-peptidic phospho-antigens (PAg) to stimulate an innate-adaptive hybrid immune response that is associated with many autoimmune diseases. Specifically, this inflammation is regulated by RORγ transcriptional control (retinoic acid receptor-related orphan receptor gamma or RAR-related orphan receptor gamma).

In particular HMBPP and IPP have been shown to regulate this process through the Vγ9Vδ2 version of the γδ TCR. Ultimately it is stimulation through this Vγ9Vδ2 TCR and other γδ TCRs that activate RORγ controlled inflammatory cytokines. Notably, it is this RORγ transcriptional control of Interleukin 17 (IL-17) and Tumor Necrosis Factor Alpha (TNFα) production that promotes autoimmune like inflammation.

The mechanism involved is further defined by several cell receptors that interact with or help present pyrophosphates to the γδ T cells. In particular, IPP and HMBPP have been shown to interact with two proteins called butyrophilin 3A1 (BTN3A1) and ecto-F1F0-ATPase (F1F0). These two membrane associated proteins are integral in the Vγ9Vδ2 TCR mediated inflammatory process. BTN3A1 acts to repress γδ TCR stimulation through a direct interaction with and repression of F1F0. When pyrophosphates such as IPP and HMBPP bind to BTN3A1 they eliminate its repressive activity and allow F1F0 to function properly and possibly play a role in direct stimulation of the γδ TCR.

While the direct display of pyrophosphates by F1F0 or BTN3A1 to the Vγ9Vδ2 TCR is not fully elucidated at this point, the evidence for direct phospho-antigen (PAg) presentation exists. Whether F1F0 acting as a presentation receptor or whether BTN3A1 plays a much bigger role in PAg presentation (in addition to its F1F0 repressor role) remains to be seen. Regardless of the method of presentation, the role of pyrophosphates in γδ TCR stimulation and the downstream effects leading to IL-17 and TNFα are clear. As such, a therapy that focuses on eliminating pyrophosphate production can have a major influence on reducing the particular immune response associated with autoimmunity.

The pyrophosphate compounds of note are produced through the mevalonate and non-mevalonate pathways of isoprenoid biosynthesis found in either the host (vertebrate) organism or associated symbiotic and pathogenic microorganisms. While the MVA pathway is found in vertebrates as well as many microorganisms, the MEP pathway (and the associated HMBPP compound) is exclusive to microorganisms. Given that the microbial derived HMBPP is over 10,000 more potent than any other known, naturally-occurring pyrophosphate, we find a mechanistic rationale for a well-known correlation between various microbial infections and particular autoimmune diseases.

While inhibition of isoprenoid biosynthesis can reduce internal pyrophosphate levels and associated inflammation there are other methods to achieve a similar effect. For example, many anti-microbials will reduce HMBPP pyrophosphate synthesis by reducing the microbial burden in the host. With this in mind, inhibition of microorganisms with an emphasis on isoprenoid biosynthesis mechanisms can be considered. Additionally, almost any anti-microbial compound will result in a coincidential reduction in MEP derived pyrophosphates with many having additive or synergistic effects when combined with isoprenoid pathway inhibitors.

However it should be noted that many antimicrobials and even some isoprenoid pathway inhibitors can elicit ajarisch-herxheimer type reaction due to a release of innate immune system triggers during cellular or microbial death. Furthermore continued production of pyrophosphates during microbial eradication can also occur. For this reason, any isoprenoid pathway inhibition or antimicrobial therapeutics can be used in conjunction with an anti-inflammatory therapeutic to prevent an acute immune stimulation during therapy.

The key to this method is to stop pyrophosphate production by any means given how these pyrophosphates drive the specific inflammation associated with autoimmune diseases.

### SUMMARY

The present disclosure provides methods and pharmaceutical compositions designed to reduce pyrophosphate drive inflammation in a patient suffering from an autoimmune disorder to treat the autoimmune disorder.

This disclosure identifies exemplary compounds that can be used in a method to treat autoimmune disorders, such as through a mechanism to inhibit pyrophosphate production within the patient. That inhibition can be directed at the patients' own metabolic pathways as well as pathways associated with symbiotic or pathogenic microorganism (the host/patient microbiome).

The method comprises administering to the subject a therapeutically effective amount of one or more pyrophosphate lowering compounds described herein. In particular, one aspect is a method of administering an isoprenoid pathway (terpenoid backbone pathway) inhibitor to reduce inflammation associated with autoimmunity.

This can be achieved through the use of compounds that inhibit any number of steps within the isoprenoid biosynthetic pathways. With many potential targets for inhibition, the most promising are those of the 1-deoxy-D-xylulose 5-phosphate synthase (also known as the DOXP synthase or Dxs), the 1-deoxy-D-xylulose 5-phosphate reductase (also known as the DOXP reductase, Dxr, or IspC), the 2-C-methyl-D-erythritol 2,4-cyclodiphosphate synthase (also known as MEcPP synthase, IspF, or MDS), the farnesyl diphosphate synthase (also known as FPPS or FDPS), and the 3-hydroxy-3-methyl-glutaryl-CoA reductase (also known as HMG-CoA reductase or HMGCR).

As a method to ensure inhibition of these pathways, inhibiting the biosynthesis of several key immune stimulating pyrophosphates such as (E)-4-hydroxy-3-methyl-but-2-enyl pyrophosphate (also known as HMBpp), isopentenyl pyrophosphate (also known as IPP), or farnesyl pyrophosphate is also described.

Another aspect of this method is to further describe specific well understood compounds that can achieve the same goal of isoprenoid biosynthesis inhibition. Therefore amelioration of autoimmune conditions and inflammation through the use of specific compounds such as fosmidomycin, fosmidomycin derivatives, thiazolo (3,2-a) pyrimidines, bisphosphonates, statins or some combination thereof is described.

Another aspect of the disclosure provides a method of treating a subject suffering from an autoimmune disease in any manner that can reduce production of the key stimulating pyrophosphates of isopentenyl pyrophosphate (IPP), (E)-4-hydroxy-3-methyl-but-2-enyl pyrophosphate (HMBpp), or farnesyl pyrophosphate. This can be achieved through the use of isoprenoid pathway inhibitors as well as anti-microbial agents. The anti-microbial agents ultimately reduce the number of MEP pathway branch (also known as the non-mevalonate, 2-C-methyl-D-erythritol 4-phosphate pathway, 1-deoxy-D-xylulose 5-phosphate pathway, or DOXP) producing microbes and use this indirect mechanistic approach.

As previously described in the previous method, this method of ameliorating autoimmune conditions and inflammation can be achieved through the use of specific compounds such as fosmidomycin, fosmidomycin derivatives, thiazolo (3,2-a) pyrimidines, bisphosphonates, statins or some combination thereof.

Another aspect of the disclosure provides a method of treating an autoimmune disorder by inhibiting the MEP pathway (also known as the non-mevalonate, 2-C-methyl-D-erythritol 4-phosphate pathway, 1-deoxy-D-xylulose 5-phosphate pathway, or DOXP). Given that the MEP pathway is one of two precursor branches of isoprenoid biosynthesis many of the same enzymes steps to be inhibitor are described. For example inhibition of 1-deoxy-D-xylulose 5-phosphate synthase (also known as the DOXP synthase or Dxs), 1-deoxy-D-xylulose 5-phosphate reductase (also known as the DOXP reductase, Dxr, or IspC), 2-C-methyl-D-erythritol 2,4-cyclodiphosphate synthase (also known as MEcPP synthase, IspF, or MDS), or farnesyl diphosphate synthase (also known as FPPS or FDPS) is described.

As with prior methods described herein, any compound that inhibits the biosynthesis of several key immune stimulating pyrophosphates of (E)-4-hydroxy-3-methyl-but-2-enyl pyrophosphate (also known as HMBpp), isopentenyl pyrophosphate (also known as IPP), or farnesyl pyrophosphate can be used.

There are two pathway branches for the isoprenoid pathway precursors and sharing of intermediates between the pathway branches of the MEP pathway and the MVA pathway (also known as the mevalonate or HMG-CoA Reductase pathway) has been known to occur. Therefore consideration of inhibition of both pathways using targets common to both pathways or through therapeutic combinations that target each pathway independently is described. Therefore another aspect of the disclosure provides a method of any preceding method where the addition of a MVA pathway inhibitor is used to treat an autoimmune disorder or related inflammation. For example, the MVA pathway inhibitor would inhibit 3-hydroxy-3-methyl-glutaryl-CoA reductase (also known as HMG-CoA reductase or HMGCR) or farnesyl diphosphate synthase (also known as FPPS or FDPS) found in his pathway branch.

Additionally, given that there is a synergistic effect between two or more isoprenoid pathway inhibitors in treating microbial infections such as parasites, the synergistic effect for multiple isoprenoid inhibitors for pyrophosphate reduction and ultimate prevention and treatment of autoimmune disease and inflammation is considered. For this reason the use of any other treatment previously described combined with an MEP pathway inhibitor is described as an important aspect. This includes any number of combinations such as two MEP pathway inhibitors, an antimicrobial compound and a MEP pathway inhibitor, or even the use of three compounds where the third compound is a MEP pathway inhibitor (and further conceivable combinations based on methods described herein).

Another important aspect is the method where an anti-microbial agent can be used in a similar combination method to treat a patient suffering from an autoimmune disease. This is described as two or more compounds that are previously described where one of the compounds is an antimicrobial. Antimicrobials can be of: any class; including antibacterials, anti-fungals, anti-mycobacterials, anti-parasitics, anti-protozoals, or anti-helmintics, any function; including anti-protein synthesis, anti-DNA replication, anti-RNA transcription, anti-RNA translation, anti-protein transferase, anti-membrane synthesis, or any class; including aminoglycosides, tetracyclines, oxazolidinones, amphenicols, pleuromutilins, macrolides, lincosamides, streptogrammins, penicillins, penems, carbapenems, cephalosporins, cephamycins, monobactams, antfolates, quinolones, nitro-imidazoles, nitrofurans, rifamycins, azoles, allylamines, hydrazides, aminoquinolines, 4-methanolquinolines, artemisinins, or sulfonamides.

In several cases, these isoprenoid pathway inhibitors and anti-microbials can exacerbate inflammation and aggravate autoimmune symptoms. This is done through the act of innate immune triggers such as PAMPs (pathogen associated molecular patterns) that act at TLRs (Toll Like Receptors) or even the acute but transient up-regulation of certain pyrophosphates intended to be inhibited. For this reason a method for the additional use of an anti-inflammatory is described as another aspect of this disclosure.

Anti-inflammatory agents considered are NSAIDS (non-steroidal anti-inflammatory drugs), corticosteroids, and small molecules and antibodies targeted to inflammatory cytokines and receptors (such as IL-17, TNFα, IL-6, IL-23, CTLA-4, CD-28, or S1P).

Given that these isoprenoids perform many metabolic functions as precursors to important cellular metabolites such as ubiquinone and cholesterol or as sources for prenyl groups in protein prenylation (and subsequent membrane localization of prenylated molecules) several other isoprenoid related functions can also be targeted. This is due to the fact that some compounds do not fit neatly into the categorization of isoprenoid pathway inhibitor, MEP pathway inhibitor, MVA pathway inhibitor, anti-microbial agent, or anti-inflammatory agent. They can only be described as another method to treat autoimmune diseases or inflammatory disorders and be used in combination with the methods previously discussed.

Therefore, an additional aspect of this disclosure includes the method of any preceding claim, further comprising administering an inhibitor of protein farnesyl transferase (also known as FTase). In a similar fashion with an anti-prenylation mechanism, another aspect of this disclosure includes the method of any preceding claim, further comprising administering an inhibitor of protein geranylgeranyl transferase (also known as GGTase). An additional aspect of this disclosure includes the method of any preceding claim, further comprising administering an inhibitor of squalene synthase (also known as SQS or farnesyl-diphosphate farnesyl transferase). Specific inhibitory compounds of these three enzymes include manumycin A, lonafarnib, tipifarnib, FTI-276, or FTI-277 (for FTase inhibition), zaragozic acid, TAK-475, or RPR 107393 (for SQS inhibition), and GGTI-298 (for GGTase inhibition).

An aspect of this disclosure is a method of any preceding claim, wherein the autoimmune disease or inflammatory disorder is any of the following diseases; ankylosing spondylitis, arthritis, rheumatoid arthritis, osteoarthritis, Chagas disease, dermatomyositis, diabetes mellitus type 1, endometriosis, Goodpasture's syndrome, Graves' disease, Guillain-Barré syndrome, Hashimoto's thyroiditis disease, Hidradenitis suppurativa, Kawasaki disease, IgA nephropathy, Idiopathic thrombocytopenic purpura, inflammatory bowel disease, Celiac's disease, Crohn's disease, eosinophilic gastroenteritis, ulcerative colitis, collagenous colitis, lymphocytic colitis, ischemic colitis, diversion colitis, Behcet's syndrome, infective colitis, indeterminate colitis interstitial cystitis, lupus, systemic lupus erythematosus, discoid lupus, drug-induced lupus, neonatal lupus, mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, temporal arteritis (also known as giant cell arteritis), vasculitis, vitiligo, Wegener's granulomatosis, alopecia areata, sarcoidosis, Addison's disease, or autoimmune hemolytic anemia.

Another aspect of the invention provides a method of treating a subject suffering from an autoimmune disease of inflammatory disorder. The method comprises administering to the subject a therapeutically effective amount of one or more compounds described herein, e.g., a compound of any preceding method.

Additional aspects of this disclosure describes the methods of administration where the combination of compounds is formulated into one pharmaceutical compound or is a combination of two or more agents delivered at the same time or at different times through various delivery methods.

Further aspects of this disclosure describe the compounds described in any preceding method using a pharmaceutically acceptable salt or solvate and may include a pharmaceutically acceptable carrier.

### BRIEF DESCRIPTION OF THE DRAWINGS

FIG. 1 depicts isoprenoid biosynthetic pathways.

FIG. 2 depicts an inflammatory pyrophosphate mechanism.

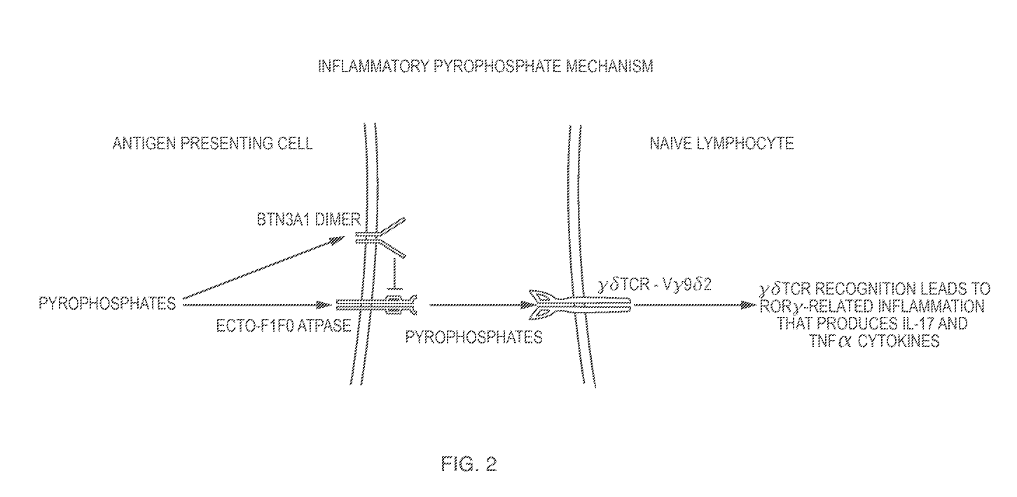

### DETAILED DESCRIPTION OF THE INVENTION

This invention provides methods and pharmaceutical compositions and formulations designed to treat autoimmune diseases. In certain embodiments, the methods involve administering to a patient in need thereof suffering from an autoimmune disease an isoprenoid pathway inhibitor. In certain embodiments, the isoprenoid pathway inhibitor is an inhibitor of the mevalonate pathway. In certain embodiments, the isoprenoid pathway inhibitor is an inhibitor of the methyl-erythritol phosphate pathway.

The use of compounds that manipulate pyrophosphate production within the isoprenoid biosynthetic pathways of both humans and microorganisms can now be identified in light of recently elucidated pathways of inflammatory signaling. Specifically, the role of pyrophosphates in triggering RORγ controlled transcription and translation of the inflammatory IL-17 and TNFα cytokines known to influence autoimmune disease is now being uncovered.

Furthermore, many of these agents when used in the methods described herein can act as anti-microbial agents. And conversely, the use of anti-microbial agents that can reduce the viability or prevalence of symbiotic or pathogenic microorganisms associated with the host can subsequently reduce pyrophosphates and autoimmune related inflammation.

Methods for accomplishing this therapeutic effect for autoimmune disorders and diseases are described. Some methods are defined by pharmaceuticals derived from one agent, while other methods require combinations of agents. As used herein, the term “effective amount” refers to the amount of a compound sufficient to effect beneficial or desired results (a therapeutic, ameliorative, inhibitory or preventative result). As used herein, the term “treating” includes any effect, e.g., lessening, reducing, modulating, ameliorating or eliminating, that results in the improvement of the condition, disease, disorder, and the like, or ameliorating a symptom thereof.

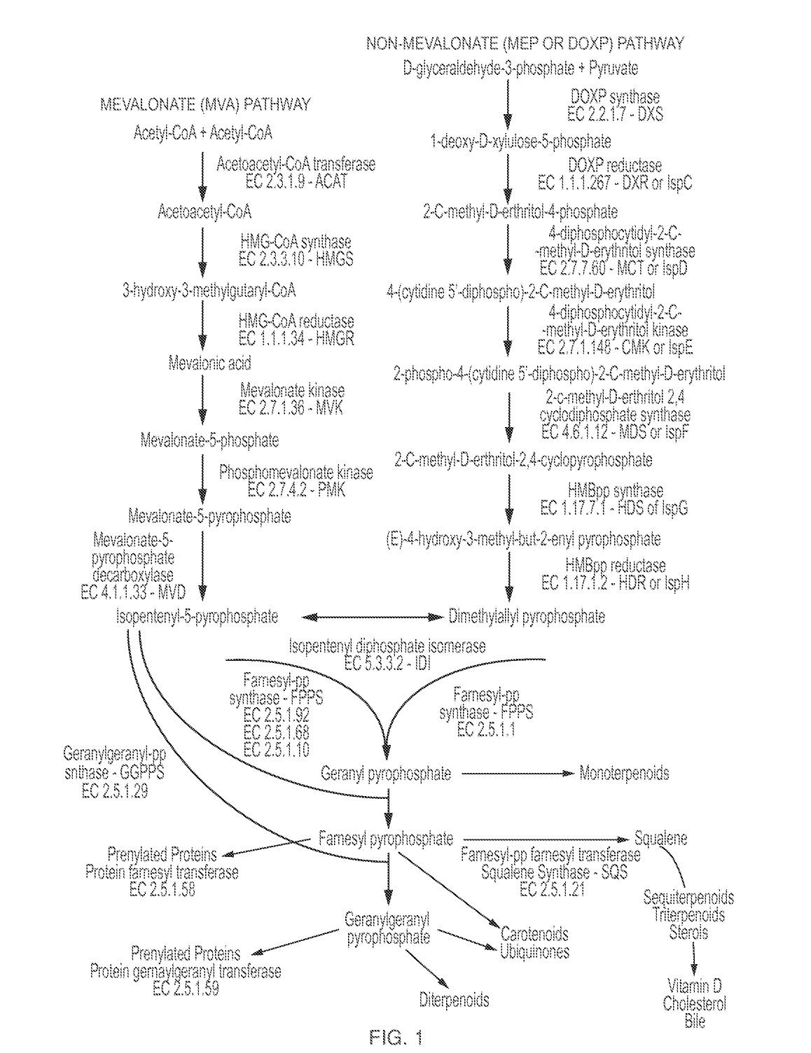

One aspect of this disclosure is a method of treating an autoimmune disorder, comprising administering to a patient in need thereof a therapeutically effective amount of an isoprenoid (also known as terpenoid backbone) pathway inhibitor to treat the autoimmune disorder. There are many enzymes of this pathway and its two branches as can be seen in FIG. 1.

The most well understood enzymes of these pathway branches include 1-deoxy-D-xylulose 5-phosphate synthase (also known as the DOXP synthase or Dxs), 1-deoxy-D-xylulose 5-phosphate reductase (also known as the DOXP reductase, Dxr, or IspC), 2-C-methyl-D-erythritol 2,4-cyclodiphosphate synthase (also known as MEcPP synthase, IspF, or MDS), farnesyl diphosphate synthase (also known as FPPS or FDPS), and 3-hydroxy-3-methyl-glutaryl-CoA reductase (also known as HMG-CoA reductase or HMGCR). These well understood enzymatic steps have known inhibitors that are already approved medicines or compounds that have advanced into clinical stage testing but have yet to be used or suggested for use in preventing or treating autoimmune diseases. Therefore one aspect of this disclosure is the use of inhibitors for these enzymatic steps as well as other isoprenoid pathway inhibitors. Ideally, pyrophosphate production is stopped early in each pathway, before production of any stimulating pyrophosphates can be achieved. Below are lists of inhibitors for many of the isoprenoid biosynthetic steps.

### Exemplary Inhibitors of the Isoprenoid Biosynthetic Pathway Include:

EC 2.2.1.7—DOXP Synthase—DXS Enzymatic Step Inhibitors
“2,3-diphospho-D-glyceric acid”
2-fluoropyruvate
“2-methyl-3,5-diphenyl-6-propylpyrazolo[1,5-a]pyrimidin-7(4H)-one”
“2-methyl-5-naphthalen-2-yl-3-phenylpyrazolo[1,5-a]pyrimidin-7(4H)-one”
“3,5-bis(4-methoxyphenyl)-2-methylpyrazolo[1,5-a]pyrimidin-7(4H)-one”
“3,5-diphenyl-2-(trifluoromethyl)pyrazolo[1,5-a]pyrimidin-7(4H)-one”
“3-(4-bromophenyl)-5-(2-methoxyphenyl)-2-methylpyrazolo[1,5-a]pyrimidin-7(4H)-one”
“3-(4-bromophenyl)-5-methyl-2-(trifluoromethyl)pyrazolo[1,5-a]pyrimidin-7(4H)-one”
“3-(4-chlorophenyl)-2-ethyl-5-(4-methoxyphenyl)pyrazolo[1,5-a]pyrimidin-7(4H)-one”
“3-(4-chlorophenyl)-2-methyl-5-phenylpyrazolo[1,5-a]pyrimidin-7(4H)-one”
“3-(4-chlorophenyl)-2-methyl-5-[[(1-phenyl-1H-tetrazol-5-yl)sulfanyl]methyl]pyrazolo[1,5-a]pyrimidin-7(4H)-one”
“3-(4-chlorophenyl)-5-(4-methoxyphenyl)-2-methylpyrazolo[1,5-a]pyrimidin-7(4H)-one”
“3-(4-chlorophenyl)-5-(methoxymethyl)-2-methylpyrazolo[1,5-a]pyrimidin-7(4H)-one”
“3-(4-chlorophenyl)-5-(methoxymethyl)pyrazolo[1,5-a]pyrimidin-7(4H)-one”
“3-(4-chlorophenyl)-5-methyl-2-(trifluoromethyl)pyrazolo[1,5-a]pyrimidin-7(4H)-one”
“3-(4-chlorophenyl)-5-methylpyrazolo[1,5-a]pyrimidin-7(4H)-one”
“3-(4-chlorophenyl)-5-[[(4-chlorophenyl)sulfanyl]methyl]-2-methylpyrazolo[1,5-a]pyrimidin-7(4H)-one”
“3-(4-fluorophenyl)-5-(4-methoxyphenyl)-2-methylpyrazolo[1,5-a]pyrimidin-7(4H)-one”
“3-(4-methoxyphenyl)-2-methyl-5-phenylpyrazolo[1,5-a]pyrimidin-7(4H)-one”
“3-(4-methoxyphenyl)-5-phenyl-2-(trifluoromethyl)pyrazolo[1,5-a]pyrimidin-7(4H)-one”
3-Fluoropyruvate
“5-(chloromethyl)-3-phenylpyrazolo[1,5-a]pyrimidin-7(4H)-one”
“5-benzyl-3-(4-chlorophenyl)-2-(trifluoromethyl)pyrazolo[1,5-a]pyrimidin-7(4H)-one”
“5-benzyl-3-(4-chlorophenyl)pyrazolo[1,5-a]pyrimidin-7(4H)-one”
“5-benzyl-3-phenyl-2-(trifluoromethyl)pyrazolo[1,5-a]pyrimidin-7(4H)-one”
“5-[[(4-chlorophenyl)sulfanyl]methyl]-2-methyl-3-phenylpyrazolo[1,5-a]pyrimidin-7(4H)-one”
“6-benzyl-3-(4-chlorophenyl)-5-methyl-2-(trifluoromethyl)pyrazolo[1,5-a]pyrimidin-7(4H)-one”
beta-fluoropyruvate
beta-Glycerophosphate
D-3-Phosphoglyceric acid
D-glyceraldehyde
D-glyceraldehyde 3-phosphate
DL-alpha-glycerophosphate
“ethyl (5-methyl-7-oxo-3-phenyl-4,7-dihydropyrazolo[1,5-a]pyrimidin-6-yl)acetate”
Fluoropyruvate
phosphonoacetohydroxamate
phosphonopropionohydroxamate
pyruvate
“methyl [3-(4-bromophenyl)-7-oxo-2-(trifluoromethyl)-4,7-dihydropyrazolo[1,5-a]pyrimidin-5-yl]acetate”

EC 1.1.1.267—DOXP Reductase—DXR or IspC Enzymatic Step Inhibitors
“(2R,3R)-2,3,4-trihydroxybutyl dihydrogen phosphate”
“(2R,3R)-4-amino-2,3-dihydroxybutyl dihydrogen phosphate”
“(2R,3S)-2,3-dihydroxy-4-(hydroxyamino)-4-oxobutyl dihydrogen phosphate”
“(2R,3S)-4-amino-2,3-dihydroxy-4-oxobutyl dihydrogen phosphate”
“(2S,3R)-2,3-dihydroxy-4-phosphonooxybutyric acid”
“(2S,3R)-dihydroxybutyramide 4-phosphate”
“(2S,3R)-methyl 2,3-dihydroxy-4-phosphonooxybutyrate”
(3-(hydroxy[(pentafluorophenyl)carbonyl]amino)propyl)phosphonic acid
(3-[hydroxy(5-oxohexanoyl)amino]propyl)phosphonic acid
(3-[hydroxy(6-phenylhexanoyl)amino]propyl)phosphonic acid
(3-[hydroxy(hexadecanoyl)amino]propyl)phosphonic acid
(3S)-hydroxypentan-2-one 5-phosphate
“(3S,4R)-3,4-dihydroxy-4-methyl-5-oxohexylphosphonic acid”
(4S)-hydroxypentan-2-one 5-phosphate
“1,1,1-trifluoro-1-deoxy-D-xylulose 5-phosphoric acid”
“1,1-difluoro-1-deoxy-D-xylulose 5-phosphoric acid”
“1,2-dideoxy-D-hexulose 6-phosphate”
“1,2-dideoxy-D-threo-3-hexulose 6-phosphate”
1-deoxy-D-xylulose 5-phosphate
1-deoxy-L-ribulose 5-phosphate
1-hydroxy-5-phenylpyridin-2(1H)-one
3-(hydroxy([(2-phenylbutanoyl)amino]acetyl)amino)propylphosphonic acid
3-(hydroxy([(3-methylbutanoyl)amino]acetyl)amino)propylphosphonic acid
3-(hydroxy([(4-phenoxybutanoyl)amino]acetyl)amino)propylphosphonic acid
3-([(1H-indol-3-yl)acetyl]amino)propylphosphonic acid
3-([2-(methoxycarbonyl)benzoyl]amino)propylphosphonic acid
3-([3-(1H-indol-3-yl)propanoyl]amino)propylphosphonic acid
3-([4-(1H-indol-3-yl)butanoyl]amino)propylphosphonic acid
3-fluoro-1-deoxy-D-xylulose-5-phosphate
“3-[(([(3,4-dimethoxyphenyl)acetyl]amino)acetyl)(hydroxy)amino]propylphosphonic acid”
3-[(2-hydroxybenzoyl)amino]propylphosphonic acid
“3-[(3,4-diethoxybenzoyl)amino]propylphosphonic acid”
“3-[(3,4-dimethoxybenzoyl)amino]propylphosphonic acid”
3-[(4-methylpentanoyl)amino]propylphosphonic acid
3-[(4-phenoxybenzoyl)amino]propylphosphonic acid
3-[([(cyclopropylcarbonyl)amino]acetyl)(hydroxy)amino]propylphosphonic acid
3-[hydroxy(([3-(trifluoromethoxy)benzoyl]amino)acetyl)amino]propylphosphonic acid
3-[hydroxy(([4-(1H-indol-3-yl)butanoyl]amino)acetyl)amino]propylphosphonic acid
4-(N-formyl-N-hydroxy-amino)-butyric acid
“4-benzylbenzene-1,2-diol”
4-fluoro-1-deoxy-D-xylulose-5-phosphate
“biphenyl-3,4-diol”
fosfoxacin
fosmidomycin
fosmidomycin analogues
FR 900098
“phosphate mono-((2S,3S)-3-fluoromethyl-2,4-dihydroxy-3-methyl-butyl) ester”
phosphoric acid mono-[2-(N-acetyl-N-hydroxy-amino)-ethyl]-ester
Pyrimethamine
sulfamic acid 2-(N-formyl-N-hydroxy-amino)-ethyl ester
“[(1-isoquinolinylamino)methylene]-1,1-bisphosphonate”
[3-(acetyl(hydroxy)amino)propyl]phosphonic acid 3-methylbutyl ester
[3-(acetyl(hydroxy)amino)propyl]phosphonic acid mono(2-naphthalen-2-yl-ethyl) ester
[3-(acetyl(hydroxy)amino)propyl]phosphonic acid mono-n-butyl ester
[3-(acetyl(hydroxy)amino)propyl]phosphonic acid mono-n-propyl ester
[3-(acetyl(hydroxy)amino)propyl]phosphonic acid monomethyl ester
[3-(acetyl(hydroxy)amino)propyl]phosphonic acid monophenethyl ester
[3-(acetyl(hydroxy)amino)propyl]phosphonic monoethyl ester
[3-(N-acetyl-N-methyl-amino)propyl]-phosphonic acid
[3-(N-formyl-N-methyl-amino)-propyl]-phosphonic acid
[4-(hydroxyamino)-4-oxobutyl]phosphonic acid
“[[(5-chloro-2-pyridinyl)amino]methylene]-1,1-bisphosphonate”
methyl jasmonate

EC 2.7.7.60—CDPME Synthase—CMS, MCT, or IspD Enzymatic Step Inhibitors
cytidine triphosphate

EC 2.7.1.148—CDPMEP Kinase—CMK or IspE Enzymatic Step Inhibitors
“ethyl [4-amino-2-oxo-5-(3-[[(2,2,2-trifluoroethyl)sulfonyl]amino]prop-1-en-1-yl)pyrimidin-1 (2H)-yl]acetate”
“N-[3-(4-amino-1-benzyl-2-oxo-1,2-dihydropyrimidin-5-yl)prop-2-en-1-yl]ethanesulfonamide”
“N-[3-[4-amino-2-oxo-1-(1H-pyrazol-5-ylmethyl)-1,2-dihydropyrimidin-5-yl]prop-2-en-1-yl]ethanesulfonamide”
“N-[3-[4-amino-2-oxo-1-(tetrahydro-2-thienyl)-1,2-dihydropyrimidin-5-yl]prop-2-en-1-yl]-1,1,1-trifluoromethanesulfonamide”
“N-[3-[4-amino-2-oxo-1-(tetrahydro-2-thienyl)-1,2-dihydropyrimidin-5-yl]prop-2-en-1-yl]-2,2,2-trifluoroethanesulfonamide”
“N-[3-[4-amino-2-oxo-1-(tetrahydro-2-thienyl)-1,2-dihydropyrimidin-5-yl]prop-2-en-1-yl]benzenesulfonamide”
“N-[3-[4-amino-2-oxo-1-(tetrahydro-2-thienyl)-1,2-dihydropyrimidin-5-yl]prop-2-en-1-yl]cyclopropanesulfonamide”
“N-[3-[4-amino-2-oxo-1-(tetrahydro-2-thienyl)-1,2-dihydropyrimidin-5-yl]prop-2-en-1-yl]ethanesulfonamide”
“N-[3-[4-amino-2-oxo-1-(tetrahydro-2-thienyl)-1,2-dihydropyrimidin-5-yl]prop-2-en-1-yl]methanesulfonamide”
“N-[3-[4-amino-2-oxo-1-(tetrahydro-2-thienyl)-1,2-dihydropyrimidin-5-yl]prop-2-en-1-yl]propane-1-sulfonamide”
“N-[3-[4-amino-2-oxo-1-(tetrahydrofuran-2-ylmethyl)-1,2-dihydropyrimidin-5-yl]prop-2-en-1-yl]-2,2,2-trifluoroethanesulfonamide”

EC 4.6.1.12—MEcPP Synthase—MCS, MDS, or IspF Enzymatic Step Inhibitors
“(+)-(S)-ethyl (2Z)-5-(1-benzofuran-2-yl)-2-(3,5-dibromo-4-hydroxybenzylidene)-7-methyl-3-oxo-2,3-dihydro-5H-[1,3]thiazolo[3,2-a]pyrimidine-6-carboxylate”
“(+/−)-(2Z)-2-(3,5-dibromo-4-hydroxybenzylidene)-5,7-dimethyl-3-oxo-2,3-dihydro-5H-[1,3]thiazolo[3,2-a]pyrimidine-6-carboxylic acid”
“(+/−)-(2Z)-2-(3,5-dibromo-4-hydroxybenzylidene)-7-methyl-3-oxo-5-(2-thienyl)-2,3-dihydro-5H-[1,3]thiazolo[3,2-a]pyrimidine-6-carboxylic acid”
“(+/−)-benzyl (2Z)-2-(3,5-dibromo-4-hydroxybenzylidene)-7-methyl-3-oxo-5-(2-thienyl)-2,3-dihydro-5H-[1,3]thiazolo[3,2-a]pyrimidine-6-carboxylate”
“(+/−)-benzyl 6-methyl-4-(2-thienyl)-2-thioxo-1,2,3,4-tetra-hydropyrimidine-5-carboxylate”
“(+/−)-ethyl (2Z)-2-(3,5-dibromo-4-hydroxybenzylidene)-5-(4-methoxyphenyl)-7-methyl-3-oxo-2,3-dihydro-5H-[1,3]thiazolo[3,2-a]pyrimidine-6-carboxylate”
“(+/−)-ethyl (2Z)-2-(3,5-dibromo-4-hydroxybenzylidene)-7-methyl-3-oxo-5-(2-thienyl)-2,3-dihydro-5H-[1,3]thiazolo[3,2-a]pyrimidine-6-carboxylate”
“(+/−)-ethyl (2Z)-2-(3-bromo-4-hydroxybenzylidene)-7-methyl-3-oxo-5-(2-thienyl)-2,3-dihydro-5H-[1,3]thiazolo[3,2-a]pyrimidine-6-carboxylate”
“(+/−)-ethyl (2Z)-2-(4-hydroxybenzylidene)-7-methyl-3-oxo-5-(2-thienyl)-2,3-dihydro-5H-[1,3]thiazolo[3,2-a]pyrimidine-6-carboxylate”
“(+/−)-ethyl (2Z)-2-[4-(acetyloxy)-3,5-dibromobenzylidene]-5-(1-benzofuran-2-yl)-7-methyl-3-oxo-2,3-dihydro-5H-[1,3]thiazolo-[3,2-a]pyrimidine-6-carboxylate”
“(+/−)-ethyl (2Z)-5-(1-benzofuran-2-yl)-2-(3,5-dibromo-4-hydroxybenzylidene)-7-methyl-3-oxo-2,3-dihydro-5H-[1,3]thiazolo[3,2-a]pyrimidine-6-carboxylate”
“(+/−)-ethyl 4-(4-methoxyphenyl)-6-methyl-2-thioxo-1,2,3,4-tetrahydro-pyrimidine-5-carboxylate”
“(−)-(R)-ethyl (2Z)-5-(1-benzofuran-2-yl)-2-(3,5-dibromo-4-hydroxybenzylidene)-7-methyl-3-oxo-2,3-dihydro-5H-[1,3]thiazolo[3,2-a]pyrimidine-6-carboxylate”
2-amino-N-hydroxy-3-(1-H-indol-3-yl)-propionimidic acid
4-amino-1-(3-O-phosphono-a-D-lyxofuranosyl)pyrimidin-2(1H)-one
“4-amino-1-[(2S,3aS,4S,6R,6aR)-2-hydroxy-6-(hydroxymethyl)-2-oxidotetrahydrofuro[3,4-d][1,3,2]dioxaphosphol-4-yl]pyrimidin-2(1H)-one”
“4-amino-1-[(4aR,6R,7R,7aS)-2,7-dihydroxy-2-oxidotetrahydro-4H-furo[3,2-d][1,3,2]dioxaphosphinin-6-yl]pyrimidin-2(1H)-one” 5-fluorocytidine
Cidofovir
Cytosine arabinoside monophosphate
N-([4-[(4-amino-2-oxopyrimidin-1(2H)-yl)methyl]-1-naphthyl]methyl)-4-chlorobenzamide
N-([4-[(4-amino-2-oxopyrimidin-1(2H)-yl)methyl]-1-naphthyl]methyl)benzamide
N-[4-[(6-aminopyridin-3-yl)amino]-3-methylbenzyl]-4-(trifluoromethyl)benzamide

EC 1.1.1.34—HMG CoA Reductase—HMGR Enzymatic Step Inhibitors—Statins
Atorvastatin or “(3R,5R)-7-[2-(4-fluorophenyl)-3-phenyl-4-(phenylcarbamoyl)-5-propan-2-ylpyrrol-1-yl]-3,5-dihydroxyheptanoic acid”
Cerivastatin or “(3R,5S,6E)-7-[4-(4-fluorophenyl)-5-(methoxymethyl)-2,6-bis(propan-2-yl)pyridin-3-yl]-3,5-dihydroxyhept-6-enoic acid”
Fluvastatin or “(3R,5S,6E)-7-[3-(4-fluorophenyl)-1-(propan-2-yl)-1H-indol-2-yl]-3,5-dihydroxyhept-6-enoic acid”
Lovastatin or “(1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl (2S)-2-methylbutanoate”
Mevastatin or “(1S,7R,8S,8aR)-8-{2-[(2R,4R)-4-hydroxy-6-oxotetrahydro-2H-pyran-2-yl]ethyl}-7-methyl-1,2,3,7,8,8a-hexahydronaphthalen-1-yl (2S)-2-methylbutanoate”
Pitavastatin or “(3R,5S,6E)-7-[2-cyclopropyl-4-(4-fluorophenyl)quinolin-3-yl]-3,5-dihydroxyhept-6-enoic acid”
Pravastatin or “(3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid”
Rosuvastatin or “(3R,5S,6E)-7-[4-(4-fluorophenyl)-2-(N-methylmethanesulfonamido)-6-(propan-2-yl)pyrimidin-5-yl]-3,5-dihydroxyhept-6-enoic acid”
Simvastatin or “(1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl 2,2-dimethylbutanoate”

EC 2.5.1.1, 2.5.1.10, 2.5.1.68, and 2.5.1.92—Farnesyl Diphosphate Synthase—FPPS Enzymatic Step Inhibitors—Bisphosphonates
Etidronate or “(1-hydroxyethan-1,1-diyl)bis(phosphonic acid)”
Clodronate or “(dichloro-phosphono-methyl)phosphonic acid”
Tiludronate or “{[(4-Chlorophenyl)thio]methylene}bis(phosphonic acid)”
Pamidronate or “(3-amino-1-hydroxypropane-1,1-diyl)bis(phosphonic acid)”
Neridronate or “(6-Amino-1-hydroxyhexane-1,1-diyl)bis(phosphonic acid)”
Olpadronate or “[3-(dimethylamino)-1-hydroxypropane-1,1-diyl]bis(phosphonic acid)”
Alendronate or “sodium [4-amino-1-hydroxy-1-(hydroxy-oxido-phosphoryl)-butyl]phosphonic acid trihydrate”
Ibandronate or “{1-hydroxy-3-[methyl(pentyl)amino]propane-1,1-diyl}bis(phosphonic acid)”
Risedronate or “(1-hydroxy-1-phosphono-2-pyridin-3-yl-ethyl)phosphonic acid”
Zoledronate or “[1-hydroxy-2-(1H-imidazol-1-yl)ethane-1,1-diyl]bis(phosphonic acid)”

Another aspect of this disclosure is based on the knowledge that inhibition of several early steps in these pathways have the ability to inhibit biosynthesis of (E)-4-Hydroxy-3-methyl-but-2-enyl pyrophosphate (also known as HMBpp) and Isopentenyl pyrophosphate (also known as IPP). Inhibition of any steps which block production of farnesyl pyrophosphate shows inhibition of both the MVA (Mevalonate) and MEP (Non-mevalonate) pathway branches and is also an aspect of this disclosure. This is an important aspect because it ensures that intermediates are not shared between pathways and it reduces intermediates used in protein prenylation. The loss of protein prenylation reduces viability of stressed cells implicated in autoimmune-like inflammation as well as microbes that could be producing MEP pathway intermediates.

In a preferred embodiment, fosmidomycin (3-(formyl hydroxy amino)propyl phosphonic acid) and its derivatives as well as other DXR inhibitors are used to regulate the MEP pathway. Exemplary compounds for use in inhibiting isoprenoid pathways are provided below:

Specific examples and their chemical structures are as follows:
Fosmidomycin, also known as 3-(Formyl-hydroxy-amino)propylphosphonic acid, having the following formula:

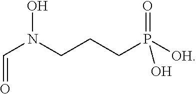 [SMILES: O=CN(O)CCCP(=O)(O)O]

A fosmidomycin derivative represented by the following formula:

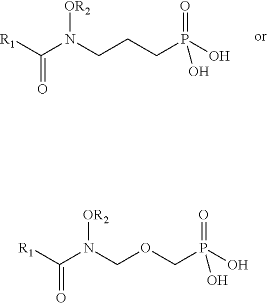

where R₁ or R₂ are independently for each occurrence:
   hydrogen
   alkyl (e.g., methyl, ethyl, propyl, and butyl)
   carboxy-substituted alkyl (e.g., a radical of butyric acid)
   optionally substituted aryl (e.g., phenyl, toluenyl, isopropyl phenyl, xylenyl, napthalenyl, biphenyl, 2-Methyl Napthalenyl, 4-Phenyltoluenyl, hydroxyl-phenyl, -phenyl-CO₂H)
   optionally substituted heteroaryl (e.g., pyridinyl, pyrimidinyl, and quinolinyl)
   guanidinyl
   acetamidinyl
Thiazolo (3,2-a) pyrimidines based on the following structure

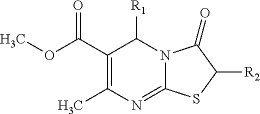

(where R₁ or R₂ can be any alkyl, hydroxyalkyl, aralkyl, heteroaralkyl, alkenyl, cyclo alkyl, aryl, heteroaryl, heterocyclic, heterocycloalkyl, amine, alkoxyl, oxo, ether, aromatic, polyaromatic, heterocyclic aromatic, guanidine, carboxamide, or amino group with further substituted halogen, organic, or inorganic molecules.
Bisphosphonates which have the basic structure

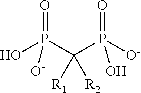

wherein R₁ and R₂ represented independently alkyl, hydroxyalkyl, aralkyl, heteroaralkyl, alkenyl, cycloalkyl, aryl, heteroaryl, heterocyclic, heterocycloalkyl, amine, alkoxyl, ether, aromatic, polyaromatic, heterocyclic aromatic, guanidine, carboxamide, or amino group with further substituted halogens, organic, or inorganic molecules; or R₁ and R₂ are taken together to form an oxo group.
   Additional specific bisphosphonic acid compounds include, for example:
Etidronate or “(1-hydroxyethan-1,1-diyl)bis(phosphonic acid)”
Clodronate or “(dichloro-phosphono-methyl)phosphonic acid”
Tiludronate or “{[(4-Chlorophenyl)thio]methylene}bis(phosphonic acid)”
Pamidronate or “(3-amino-1-hydroxypropane-1,1-diyl)bis(phosphonic acid)”
Neridronate or “(6-Amino-1-hydroxyhexane-1,1-diyl)bis(phosphonic acid)”
Olpadronate or “[3-(dimethylamino)-1-hydroxypropane-1,1-diyl]bis(phosphonic acid)”
Alendronate or “sodium [4-amino-1-hydroxy-1-(hydroxy-oxido-phosphoryl)-butyl]phosphonic acid trihydrate”
Ibandronate or “{1-hydroxy-3-[methyl(pentyl)amino]propane-1,1-diyl}bis(phosphonic acid)”
Risedronate or “(1-hydroxy-1-phosphono-2-pyridin-3-yl-ethyl)phosphonic acid”
Zoledronate or “[1-hydroxy-2-(1H-imidazol-1-yl)ethane-1,1-diyl]bis(phosphonic acid)” Statins
Atorvastatin or “(3R,5R)-7-[2-(4-fluorophenyl)-3-phenyl-4-(phenylcarbamoyl)-5-propan-2-ylpyrrol-1-yl]-3,5-dihydroxyheptanoic acid”
Cerivastatin or “(3R,5S,6E)-7-[4-(4-fluorophenyl)-5-(methoxymethyl)-2,6-bis(propan-2-yl)pyridin-3-yl]-3,5-dihydroxyhept-6-enoic acid”
Fluvastatin or “(3R,5S,6E)-7-[3-(4-fluorophenyl)-1-(propan-2-yl)-1H-indol-2-yl]-3,5-dihydroxyhept-6-enoic acid”
Lovastatin or “(1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl (2S)-2-methylbutanoate”
Mevastatin or “(1S,7R,8S,8aR)-8-{2-[(2R,4R)-4-hydroxy-6-oxotetrahydro-2H-pyran-2-yl]ethyl}-7-methyl-1,2,3,7,8,8a-hexahydronaphthalen-1-yl (2S)-2-methylbutanoate”
Pitavastatin or “(3R,5S,6E)-7-[2-cyclopropyl-4-(4-fluorophenyl)quinolin-3-yl]-3,5-dihydroxyhept-6-enoic acid”
Pravastatin or “(3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid”
Rosuvastatin or “(3R,5S,6E)-7-[4-(4-fluorophenyl)-2-(N-methylmethanesulfonamido)-6-(propan-2-yl)pyrimidin-5-yl]-3,5-dihydroxyhept-6-enoic acid”
Simvastatin or “(1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl 2,2-dimethylbutanoate”

Another aspect of this disclosure is a method of reducing the amount of a pyrophosphate selected from Isopentenyl Pyrophosphate (IPP), (E)-4-Hydroxy-3-methyl-but-2-enyl pyrophosphate (HMBpp), or Farnesyl Pyrophosphate in a patient suffering from an autoimmune disorder, comprising administering to a patient in need thereof an effective amount of an agent that directly or indirectly reduces the amount of a pyrophosphate selected from Isopentenyl Pyrophosphate (IPP), (E)-4-Hydroxy-3-methyl-but-2-enyl pyrophosphate (HMBpp), or Farnesyl Pyrophosphate in the patient.

This aspect describes a method focusing on the actual reduction of the immune stimulating pyrophosphates. Like the methods above it includes various pathway isoprenoid pathway inhibitors and specific compounds or structures (such as fosmidomycin, fosmidomycin derivatives, thiazolo (3,2-a) pyrimidines, bisphosphonates, or statins).

Another aspect of this disclosure describes a method of treating an autoimmune disorder, comprising administering to a patient in need thereof a therapeutically effective amount of a MEP pathway (also known as the non-mevalonate, 2-C-methyl-D-erythritol 4-phosphate pathway, 1-deoxy-D-xylulose 5-phosphate pathway, or DOXP) inhibitor to treat the autoimmune disorder.

The focus of this can be any number of enzyme targets, with the most promising being the 1-deoxy-D-xylulose 5-phosphate synthase (also known as the DOXP synthase or Dxs), the 1-deoxy-D-xylulose 5-phosphate reductase (also known as the DOXP reductase, Dxr, or IspC), the 2-C-methyl-D-erythritol 2,4-cyclodiphosphate synthase (also known as MEcPP synthase, IspF, or MDS), the farnesyl diphosphate synthase (also known as FPPS or FDPS). Any of these targets will help prevent the production key pyrophosphates (HMBPP, IPP, and Farnesyl pyrophosphate) that are associated with autoimmune related inflammation.

There are synergistic effects of multiple isoprenoid pathway inhibitors in reducing pyrophosphates as well as producing an anti-microbial effect. Therefore another aspect of this disclosure is a method where MVA inhibitors are used in combination with any previously mentioned therapeutic is described. In preferred embodiments, the MVA pathway steps focus on inhibition of the 3-hydroxy-3-methyl-glutaryl-CoA reductase (also known as HMG-CoA reductase or HMGCR) or the farnesyl diphosphate synthase (also known as FPPS or FDPS).

Furthermore, additional compounds with synergistic effects to be considered are additional MEP pathway inhibitors such as the 1-deoxy-D-xylulose 5-phosphate synthase (also known as the DOXP synthase or Dxs), the 1-deoxy-D-xylulose 5-phosphate reductase (also known as the DOXP reductase, Dxr, or IspC), the 2-C-methyl-D-erythritol 2,4-cyclodiphosphate synthase (also known as MEcPP synthase, IspF, or MDS), the farnesyl diphosphate synthase (also known as FPPS or FDPS). This provides many potential therapeutic options to reduce isoprenoid biosynthesis and production of pyrophosphate intermediates, such as dual MEP pathway inhibitors.

As mentioned above, another preferred embodiment would be the use of anti-microbial agents in combination with any of the aforementioned compositions. As with the use of MEP pathway inhibitors in treating microbial infections, the synergistic effect with classic antibiotics can be another method to squelch production of isoprenoid biosynthesis or pyrophosphate production produced by infectious microorganisms.

Antimicrobials that are considered are anti-amoeba, anti-protozoal, anti-bacterial, or anti-fungal in nature. Following along the classic designation of antimicrobials by ATC (Anatomical Therapeutic hemical classification system maintained by WHO (the World Health Organization). Based on this view for combination with anti-microbials, the list includes:

Antimicrobials (with Associated ATC Codes)
J01A Tetracyclines, J01AA Tetracyclines; J01AA01 Demeclocycline, J01AA02 Doxycycline, J01AA03 Chlortetracycline, J01AA04 Lymecycline, J01AA05 Metacycline, J01AA06 Oxytetracycline, J01AA07 Tetracycline, J01AA08 Minocycline, J01AA09 Rolitetracycline, J01AA10 Penimepicycline, J01AA11 Clomocycline, J01AA12 Tigecycline
J01B Amphenicols, J01BA Amphenicols; J01BA01 Chloramphenicol, J01BA02 Thiamphenicol
J01C Beta-lactam antibacterials, penicillins
J01CA Penicillins with extended spectrum; J01CA01 Ampicillin, J01CA02 Pivampicillin, J01CA03 Carbenicillin, J01CA04 Amoxicillin, J01CA05 Carindacillin, J01CA06 Bacampicillin, J01CA07 Epicillin, J01CA08 Pivmecillinam, J01CA09 Azlocillin, J01CA10 Mezlocillin, J01CA11 Mecillinam, J01CA12 Piperacillin, J01CA13 Ticarcillin, J01CA14 Metampicillin, J01CA15 Talampicillin, J01CA16 Sulbenicillin, J01CA17 Temocillin, J01CA18 Hetacillin, J01CA19 Aspoxicillin
J01CE Beta-lactamase-sensitive penicillins; J01CE01 Benzylpenicillin, J01CE02 Phenoxymethylpenicillin, J01CE03 Propicillin, J01CE04 Azidocillin, J01CE05 Pheneticillin, J01CE06 Penamecillin, J01CE07 Clometocillin, J01CE08 Benzathine benzylpenicillin, J01CE09 Procaine benzylpenicillin, J01CE10 Benzathine phenoxymethylpenicillin
J01CF Beta-lactamase-resistant penicillins; J01CF01 Dicloxacillin, J01CF02 Cloxacillin, J01CF03 Methicillin, J01CF04 Oxacillin, J01CF05 Flucloxacillin, J01CF06 Nafcillin
J01CG Beta-lactamase inhibitors; J01CG01 Sulbactam, J01CG02 Tazobactam
J01D Other beta-lactam antibacterials
J01DB First-generation cephalosporins; J01DB01 Cefalexin, J01DB02 Cefaloridine, J01DB03 Cefalotin, J01DB04 Cefazolin, J01DB05 Cefadroxil, J01DB06 Cefazedone, J01DB07 Cefatrizine, J01DB08 Cefapirin, J01DB09 Cefradine, J01DB10 Cefacetrile, J01DB11 Cefroxadine, J01DB12 Ceftezole
J01DC Second-generation cephalosporins; J01DC01 Cefoxitin, J01DC02 Cefuroxime, J01DC03 Cefamandole, J01DC04 Cefaclor, J01DC05 Cefotetan, J01DC06 Cefonicide, J01DC07 Cefotiam, J01DC08 Loracarbef, J01DC09 Cefmetazole, J01DC10 Cefprozil, J01DC11 Ceforanide, J01DC12 Cefminox, J01DC13 Cefbuperazone, J01DC14 Flomoxef
J01DD Third-generation cephalosporins; J01DD01 Cefotaxime, J01DD02 Ceftazidime, J01DD03 Cefsulodin, J01DD04 Ceftriaxone, J01DD05 Cefmenoxime, J01DD06 Latamoxef, J01DD07 Ceftizoxime, J01DD08 Cefixime, J01DD09 Cefodizime, J01DD10 Cefetamet, J01DD11 Cefpiramide, J01DD12 Cefoperazone, J01DD13 Cefpodoxime, J01DD14 Ceftibuten, J01DD15 Cefdinir, J01DD16 Cefditoren, J01DD17 Cefcapene
J01DE Fourth-generation cephalosporins; J01DE01 Cefepime, J01DE02 Cefpirome, J01DE03 Cefozopran
J01DF Monobactams; J01DF01 Aztreonam, J01DF02 Carumonam
J01DH Carbapenems; J01DH02 Meropenem, J01DH03 Ertapenem, J01DH04 Doripenem, J01DH05 Biapenem
J01DI Other cephalosporins and penems; J01DI01 Ceftobiprole medocaril, J01DI02 Ceftaroline fosamil, J01DI03 Faropenem
J01E Sulfonamides and trimethoprim
J01EA Trimethoprim and derivatives; J01EA01 Trimethoprim, J01EA02 Brodimoprim, J01EA03 Iclaprim
J01EB Short-acting sulfonamides; J01EB01 Sulfaisodimidine, J01EB02 Sulfamethizole, J01EB03 Sulfadimidine, J01EB04 Sulfapyridine, J01EB05 Sulfafurazole, J01EB06 Sulfanilamide, J01EB07 Sulfathiazole, J01EB08 Sulfathiourea
J01EC Intermediate-acting sulfonamides; J01EC01 Sulfamethoxazole, J01EC02 Sulfadiazine, J01EC03 Sulfamoxole
J01ED Long-acting sulfonamides; J01ED01 Sulfadimethoxine, J01ED02 Sulfalene, J01ED03 Sulfametomidine, J01ED04 Sulfametoxydiazine, J01ED05 Sulfamethoxypyridazine, J01ED06 Sulfaperin, J01ED07 Sulfamerazine, J01ED08 Sulfaphenazole, J01ED09 Sulfamazon
“J01F Macrolides, lincosamides and streptogramins”
J01FA Macrolides; J01FA01 Erythromycin, J01FA02 Spiramycin, J01FA03 Midecamycin, J01FA05 Oleandomycin, J01FA06 Roxithromycin, J01FA07 Josamycin, J01FA08 Troleandomycin, J01FA09 Clarithromycin, J01FA10 Azithromycin, J01FA11 Miocamycin, J01FA12 Rokitamycin, J01FA13 Dirithromycin, J01FA14 Flurithromycin, J01FA15 Telithromycin
J01FF Lincosamides; J01FF01 Clindamycin, J01FF02 Lincomycin
J01FG Streptogramins; J01FG01 Pristinamycin, J01FG02 Quinupristin/dalfopristin
J01G Aminoglycoside antibacterials
J01GA Streptomycins; J01GA01 Streptomycin, J01GA02 Streptoduocin
J01 GB Other aminoglycosides; J01GB01 Tobramycin, J01GB03 Gentamicin, J01GB04 Kanamycin, J01GB05 Neomycin, J01GB06 Amikacin, J01GB07 Netilmicin, J01GB08 Sisomicin, J01GB09 Dibekacin, J01GB10 Ribostamycin, J01GB11 Isepamicin, J01GB12 Arbekacin, J01GB13 Bekanamycin
J01M Quinolone antibacterials
J01MA Fluoroquinolones; J01MA01 Ofloxacin, J01MA02 Ciprofloxacin, J01MA03 Pefloxacin, J01MA04 Enoxacin, J01MA05 Temafloxacin, J01MA06 Norfloxacin, J01MA07 Lomefloxacin, J01MA08 Fleroxacin, J01MA09 Sparfloxacin, J01MA10 Rufloxacin, J01MA1 Grepafloxacin, J01MA12 Levofloxacin, J01MA13 Trovafloxacin, J01MA14 Moxifloxacin, J01MA15 Gemifloxacin, J01MA16 Gatifloxacin, J01MA17 Prulifloxacin, J01MA18 Pazufloxacin, J01MA19 Garenoxacin, J01MA21 Sitafloxacin
J01MB Other quinolones; J01MB01 Rosoxacin, J01MB02 Nalidixic acid, J01MB03 Piromidic acid, J01MB04 Pipemidic acid, J01MB05 Oxolinic acid, J01MB06 Cinoxacin, J01MB07 Flumequine
J01X Other antibacterials
J01XA Glycopeptide antibacterials; J01XA01 Vancomycin, J01XA02 Teicoplanin, J01XA03 Telavancin, J01XA04 Dalbavancin, J01XA05 Oritavancin
J01XB Polymyxins; J01XB01 Colistin, J01XB02 Polymyxin B
J01XC Steroid antibacterials; J01XC01 Fusidic acid
J01XD Imidazole derivatives; J01XD01 Metronidazole, J01XD02 Tinidazole, J01XD03 Ornidazole
J01XE Nitrofuran derivatives; J01XE01 Nitrofurantoin, J01XE02 Nifurtoinol
J01XX Other antibacterials; J01XX01 Fosfomycin, J01XX02 Xibornol, J01XX03 Clofoctol, J01XX04 Spectinomycin, J01XX05 Methenamine, J01XX06 Mandelic acid, J01XX07 Nitroxoline, J01XX08 Linezolid, J01XX09 Daptomycin, J01XX10 Bacitracin
J02A Antimycotics for systemic use
J02AA Antibiotics; J02AA01 Amphotericin B, J02AA02 Hachimycin
J02AB Imidazole derivatives; J02AB01 Miconazole, J02AB02 Ketoconazole, QJ02AB90 Clotrimazole
J02AC Triazole derivatives; J02AC01 Fluconazole, J02AC02 Itraconazole, J02AC03 Voriconazole, J02AC04 Posaconazole
J02AX Other antimycotics for systemic use; J02AX01 Flucytosine, J02AX04 Caspofungin, J02AX05 Micafungin, J02AX06 Anidulafungin
J04A Drugs for treatment of tuberculosis
J04AA Aminosalicylic acid and derivatives; J04AA01 Aminosalicylic acid, J04AA02 Sodium aminosalicylate, J04AA03 Calcium aminosalicylate
J04AB Antibiotics; J04AB01 Cycloserine, J04AB02 Rifampicin, J04AB03 Rifamycin, J04AB04 Rifabutin, J04AB05 Rifapentin, J04AB30 Capreomycin
J04AC Hydrazides; J04AC01 Isoniazid
J04AD Thiocarbamide derivatives; J04AD01 Protionamide, J04AD02 Tiocarlide, J04AD03 Ethionamide
J04AK Other drugs for treatment of tuberculosis; J04AK01 Pyrazinamide, J04AK02 Ethambutol, J04AK03 Terizidone, J04AK04 Morinamide
J04B Drugs for treatment of lepra
J04BA Drugs for treatment of lepra; J04BA01 Clofazimine, J04BA02 Dapsone, J04BA03 Aldesulfone sodium
P01A Agents against amoebiasis and other protozoal diseases
P01AA Hydroxyquinoline derivatives; P01AA01 Broxyquinoline, P01AA02 Clioquinol, P01AA04 Chlorquinaldol, P01AA05 Tilbroquinol
P01AB Nitroimidazole derivatives; P01AB01 Metronidazole, P01AB02 Tinidazole, P01AB03 Ornidazole
P01AB04 Azanidazole, P01AB05 Propenidazole, P01AB06 Nimorazole, P01AB07 Secnidazole
P01AC Dichloroacetamide derivatives; P01AC01 Diloxanide, P01AC02 Clefamide, P01AC03 Etofamide, P01AC04 Teclozan
P01AR Arsenic compounds; P01AR01 Arsthinol, P01AR02 Difetarsone, P01AR03 Glycobiarsol
P01AX Other agents against amoebiasis and other protozoal diseases; P01AX01 Chiniofon, P01AX02 Emetine, P01AX04 Phanquinone, P01AX05 Mepacrine, P01AX06 Atovaquone, P01AX07 Trimetrexate, P01AX08 Tenonitrozole, P01AX09 Dehydroemetine, P01AX10 Fumagillin
P01B Antimalarials
P01BA Aminoquinolines; P01BA01 Chloroquine, P01BA02 Hydroxychloroquine, P01BA03 Primaquine, P01BA06 Amodiaquine
P01BB Biguanides; P01BB01 Proguanil, P01BB02 Cycloguanil embonate
P01BC Methanolquinolines; P01BC01 Quinine, P01BC02 Mefloquine
P01BD Diaminopyrimidines; P01BD01 Pyrimethamine
P01BE Artemisinin and derivatives; P01BE01 Artemisinin, P01BE02 Artemether, P01BE03 Artesunate, P01BE04 Artemotil, P01BE05 Artenimol
P01BX Other antimalarials; P01BX01 Halofantrine
P01C Agents against leishmaniasis and trypanosomiasis
P01CA Nitroimidazole derivatives; P01CA02 Benznidazole
P01CB Antimony compounds; P01CB01 Meglumine antimonite, P01CB02 Sodium stibogluconate
P01CC Nitrofuran derivatives; P01CC01 Nifurtimox, P01CC02 Nitrofural
P01CD Arsenic compounds; P01CD01 Melarsoprol, P01CD02 Acetarsol
P01CX Other agents against leishmaniasis and trypanosomiasis; P01CX01 Pentamidine isethionate, P01CX02 Suramin sodium, P01CX03 Eflornithine

Another aspect of this disclosure describes a method of any preceding claim, further comprising administering an anti-inflammatory agent. This would prevent any inflammatory conditions arising from the anti-microbial nature of isoprenoid pathway inhibitors or other previously described therapeutics. Key inflammatory classes would be those that target autoimmune related inflammation such as the IL-17 or TNFα cytokines. However, other inflammatory cytokines that would signal a Jarisch-Herxheimer type of reaction are also considered for anti-inflammatory targeting. These include targeting of IL-6, IL-23, CD-28. Many of the anti-inflammatories can be antibody type therapies (MAb).

Anti-Inflammatory Agents (with Associated ATC Codes)
L04A Immunosuppressants;
L04AA Selective immunosuppressants; L04AA02 Muromonab-CD3, L04AA03 Antilymphocyte immunoglobulin (horse), L04AA04 Antithymocyte immunoglobulin (rabbit), L04AA06 Mycophenolic acid, L04AA10 Sirolimus, L04AA13 Leflunomide, L04AA15 Alefacept, L04AA18 Everolimus, L04AA19 Gusperimus, L04AA21 Efalizumab, L04AA22 Abetimus, L04AA23 Natalizumab, L04AA24 Abatacept, L04AA25 Eculizumab, L04AA26 Belimumab, L04AA27 Fingolimod, L04AA28 Belatacept, L04AA29 Tofacitinib
L04AB Tumor necrosis factor alpha (TNF-\grave{α}) inhibitors; L04AB01 Etanercept, L04AB02 Infliximab, L04AB03 Afelimomab, L04AB04 Adalimumab, L04AB05 Certolizumab pegol, L04AB06 Golimumab
L04AC Interleukin inhibitors; L04AC01 Daclizumab, L04AC02 Basiliximab, L04AC03 Anakinra, L04AC04 Rilonacept, L04AC05 Ustekinumab, L04AC06 Mepolizumab, L04AC07 Tocilizumab, L04AC08 Canakinumab, L04AC09 Briakinumab, L04AC10 Secukinumab
L04AD Calcineurin inhibitors; L04AD01 Ciclosporin, L04AD02 Tacrolimus, L04AD03 Voclosporin
L04AX Other immunosuppressants; L04AX01 Azathioprine, L04AX02 Thalidomide, L04AX03 Methotrexate, L04AX04 Lenalidomide, L04AX05 Pirfenidone, H02A Corticosteroids for systemic use, H02AA Mineralocorticoids, H02AA01 Aldosterone, H02AA02 Fludrocortisone, H02AA03 Desoxycortone
H02AB Glucocorticoids; H02AB01 Betamethasone, H02AB02 Dexamethasone, H02AB03 Fluocortolone
H02AB04 Methylprednisolone, H02AB05 Paramethasone, H02AB06 Prednisolone, H02AB07 Prednisone
H02AB08 Triamcinolone, H02AB09 Hydrocortisone, H02AB10 Cortisone, H02AB11 Prednylidene
H02AB12 Rimexolone, H02AB13 Deflazacort, H02AB14 Cloprednol, H02AB15 Meprednisone, H02AB17 Cortivazol
H02C Antiadrenal preparations
H02CA Anticorticosteroids; H02CA01 Trilostane
N06AX12 Bupropion

Another aspect is a method of any preceding claim, further comprising administering an inhibitor of the protein farnesyl transferase (also known as FTase) enzyme or enzymatic step that diverges from of the isoprenoid biosynthetic pathway. This is important for RAS prenylation and membrane association of particular proteins. Inhibiting this plays a major role in cellular processes that are controlled by isoprenoids. It also plays a role in inhibiting growth and functionality of many microorganisms, so inhibition can ultimately, although indirectly, inhibit pyrophosphate production.

In particular, protein farnesyl transfer inhibitors include:

EC 2.5.1.58—Farnesyl Diphosphate Transferase—FT Enzymatic Step Inhibitors
(+)-6-(camphorquinone-10-sulfonamido)-hexanoic acid
(−)-6-(camphorquinone-10-sulfonamido)-hexanoic acid
“(1aR,2S,3aS,6aR,7aR)-2,6-dimethyl-1a-[(1E)-3-oxobut-1-en-1-yl]octahydro-5H-oxireno[4,5]cyclohepta[1,2-b]furan-5-one”
“(1aR,2S,3 aS,6aR,7aR)-2-methyl-6-methylidene-1a-[(1E)-3-oxobut-1-en-1-yl]octahydro-5H-oxireno[4,5]cyclohepta[1,2-b]furan-5-one”
“(1 aS,2S,3aS,6aR,7aS)-2,6-dimethyl-1a-[(1E)-3-oxobut-1-en-1-yl]octahydro-5H-oxireno[4,5]cyclohepta[1,2-b]furan-5-one”
“(1 aS,2S,3aS,6aR,7aS)-2-methyl-6-methylidene-1a-[(1E)-3-oxobut-1-en-1-yl]octahydro-5H-oxireno[4,5]cyclohepta[1,2-b]furan-5-one”
“(1R,4aR,5R,7S)-5-[[(1R,4aR,8aS)-8a-(5,7-dihydroxy-4-oxo-4H-chromen-2-yl)-6-methoxy-5,8-dioxo-1,4,4a,5,8,8a-hexahydronaphthalen-1-yl]methyl]-7-hydroxy-1,4a-dimethyl-6-methylidenedecahydronaphthalene-1-carbaldehyde”
“(1R,4aR,5R,7S)-5-[[(1R,4aR,8aS)-8a-(5,7-dihydroxy-4-oxo-4H-chromen-2-yl)-6-methoxy-5,8-dioxo-1,4,4a,5,8,8a-hexahydronaphthalen-1-yl]methyl]-7-hydroxy-1,4a-dimethyl-6-methylidenedecahydronaphthalene-1-carboxylic acid”
“(2E)-2-[(2E,6E)-3,7,11-trimethyldodeca-2,6,10-trien-1-ylidene]butanedioic acid”
“(2E)-3-[(3aR,7S,8aS)-3,7-dimethyl-2-oxo-3,3a,4,7,8,8a-hexahydro-2H-cyclohepta[b]furan-6-yl]-1-methylprop-2-en-1-yl acetate”
“(2E,4E,8E)-5,9,13-trimethyltetradeca-2,4,8,12-tetraenoic acid”
“(2Z)-3-(1H-imidazol-2-yl)-5-methylhexa-2,4-dienoic acid”
“(2Z)-5-methyl-3-(1-methyl-1H-imidazol-2-yl)hexa-2,4-dienoic acid”
“(2Z,4E)-5,9-dimethyl-3-(1-methyl-1H-imidazol-2-yl)deca-2,4,8-trienoic acid”
“(2Z,4E,8E)-5,9,13-trimethyl-3-(1-methyl-1H-imidazol-2-yl)tetradeca-2,4,8,12-tetraenoic acid”
“(2Z,6E)-3-(3-methylbut-2-enyl)-7,11-dimethyldodeca-2,6,10-triene monophosphate”
“(2Z,6E)-3-allyl-7,11-dimethyldodeca-2,6,10-trien-1-yl 5-nitrofurfuryl N-methyl-N-(4-chlorobutyl)phosphoramidate”
“(2Z,6E)-3-allyl-7,11-dimethyldodeca-2,6,10-triene monophosphate”
“(2Z,6E)-3-tert-butyl-7,11-dimethyldodeca-2,6,10-trien-1-yl 5-nitrofurfuryl N-methyl-N-(4-chlorobutyl)phosphoramidate”
“(2Z,6E)-3-tert-butyl-7,11-dimethyldodeca-2,6,10-triene monophosphate”
“(2Z,6E,10E)-3-(3-methyl-1-but-2-enyl)-7,11-dimethyldodeca-2,6,10-trien-1-yl 5-nitrofurfuryl N-methyl-N-(4-chlorobutyl)phosphoramidate”
“(3aR,7S,8aS)-3,7-dimethyl-6-[(1E)-3-oxobut-1-en-1-yl]-3,3a,4,7,8,8a-hexahydro-2H-cyclohepta[b]furan-2-one”
“(3aR,7S,8aS)-6-[(1E)-3-hydroxybut-1-en-1-yl]-3,7-dimethyl-3,3a,4,7,8,8a-hexahydro-2H-cyclohepta[b]furan-2-one”
“(3aR,7S,8aS)-7-methyl-3-methylidene-6-[(1E)-3-oxobut-1-en-1-yl]-3,3a,4,7,8,8a-hexahydro-2H-cyclohepta[b]furan-2-one”
“(3aR,8S,9aS)-6-acetyl-3,8-dimethyl-3,3a,4,8,9,9a-hexahydro-2H-cyclohepta[1,2-b:4,5-b′]difuran-2-one”
“(3R,3aR,7S,8aS)-3,7-dimethyl-6-(3-oxobutyl)-3,3a,4,7,8,8a-hexahydro-2H-cyclohepta[b]furan-2-one”
“(3R,3aR,7S,8aS)-6-(3-hydroxybutyl)-3,7-dimethyl-3,3a,4,7,8,8a-hexahydro-2H-cyclohepta[b]furan-2-one”
“(4aS,5R,8aR)-4a-(5,7-dihydroxy-4-oxo-4H-chromen-2-yl)-2-methoxy-5-[[(1S,3S,7S,8aS)-1,3,7-trihydroxy-5,5,8a-trimethyl-2-methylidenedecahydronaphthalen-1-yl]methyl]-4a,5,8,8a-tetrahydronaphthalene-1,4-dione”
“(4aS,5R,8aR)-4a-(5,7-dihydroxy-4-oxo-4H-chromen-2-yl)-2-methoxy-6-methyl-5-[[(1R,8aS)-5,5,8a-trimethyl-2-methylidene-3-oxodecahydronaphthalen-1-yl]methyl]-4a,5,8,8a-tetrahydronaphthalene-1,4-dione”
“(4aS,5R,8aR)-4a-(5,7-dihydroxy-4-oxo-4H-chromen-2-yl)-5-[[(1R,3S,5R,8aS)-3-hydroxy-5-(hydroxymethyl)-5,8a-dimethyl-2-methylidenedecahydronaphthalen-1-yl]methyl]-2-methoxy-4a,5,8,8a-tetrahydronaphthalene-1,4-dione”
“(4aS,5R,8aR)-4a-(5,7-dihydroxy-4-oxo-4H-chromen-2-yl)-5-[[(1R,3S,7S,8aS)-3,7-dihydroxy-5,5,8a-trimethyl-2-methylidenedecahydronaphthalen-1-yl]methyl]-2-methoxy-4a,5,8,8a-tetrahydronaphthalene-1,4-dione”
“(4aS,5R,8aR)-4a-(5,7-dihydroxy-4-oxo-4H-chromen-2-yl)-5-[[(1R,3S,8aS)-3-hydroxy-5,5,8a-trimethyl-2-methylidenedecahydronaphthalen-1-yl]methyl]-2-methoxy-4a,5,8,8a-tetrahydronaphthalene-1,4-dione”
“(4aS,5R,8aR)-4a-(5-hydroxy-7-methoxy-4-oxo-4H-chromen-2-yl)-2-methoxy-6-methyl-5-[[(1R,8aS)-5,5,8a-trimethyl-2-methylidene-3-oxodecahydronaphthalen-1-yl]methyl]-4a,5,8,8a-tetrahydronaphthalene-1,4-dione”
“(4aS,5S,8aR)-4a-(5,7-dihydroxy-4-oxo-4H-chromen-2-yl)-5-[[(1R,2R,7S,8aS)-2,7-dihydroxy-2,5,5,8a-tetramethyldecahydronaphthalen-1-yl]methyl]-6-hydroxy-2-methoxy-4a,5,8,8a-tetrahydronaphthalene-1,4-dione”
“(4E)-5,9-dimethyl-3-(1-methyl-1H-imidazol-2-yl)deca-4,8-dienoic acid”
“(4E,8E)-2-(ethoxycarbonyl)-5,9,13-trimethyl-2-[3-(1-methyl-1H-imidazol-2-yl)propyl]tetradeca-4,8,12-trienoic acid”
“(4E,8E)-5,9,13-trimethyl-3-(1-methyl-1H-imidazol-2-yl)tetradeca-4,8,12-trienoic acid”
“(4E,8E)-5,9,13-trimethyl-N-(phenylsulfonyl)tetradeca-4,8,12-trienamide”
“(4E,8E)-5,9,13-trimethyltetradeca-4,8,12-trienamide”
“(4E,8E)-5,9,13-trimethyltetradeca-4,8,12-trienoic acid”
“(4E,8E)-N-hydroxy-5,9,13-trimethyltetradeca-4,8,12-trienamide”
“(6E,10E)-7,11,15-trimethyl-3-oxohexadeca-6,10,14-trienoic acid”
“(7E,11E)-8,12,16-trimethyl-4-oxoheptadeca-7,11,15-trienoic acid”
“(E,E)-8-O-(3-benzoylbenzyl)-3,7-dimethyl-2,6-octadiene 1-diphosphate”
“(E,E)-8-O-(4-benzoylbenzyl)-3,7-dimethyl-2,6-octadiene 1-diphosphate”
“(S)—N-(4-(3,4-dichlorophenoxy)benzyl)-6-(1H-indol-3-yl)piperazine-2,5-dione”
“(S)—N-(4-(3-chlorophenoxy)benzyl)-6-(1H-indol-3-yl)piperazine-2,5-dione”
“1,4,8,10-tetrahydroxy-6-[(1E)-3-[(3S,8aS)-3-hydroxy-5,5,8a-trimethyl-2-methylidenedecahydronaphthalen-1-yl]-1-methylprop-1-en-1-yl]-5,6-dihydro-7H-benzo[c]xanthen-7-one”
“1,5-dimethyl-1H-imidazole-4-sulfonic acid (5-bromo-2-fluorobenzyl)-[6-cyano-1-(3-methyl-3H-imidazol-4-ylmethyl)-1,2,3,4-tetrahydro-quinolin-3-yl]-amide”
“1,5-dimethyl-1H-imidazole-4-sulfonic acid [6-cyano-1-(3-methyl-3H-imidazol-4-ylmethyl)-1,2,3,4-tetrahydro-quinolin-3-yl]-(4-ethanesulfonyl-benzyl)-amide”
“1,5-dimethyl-1H-imidazole-4-sulfonic acid [6-cyano-1-(3-methyl-3H-imidazol-4-ylmethyl)-1,2,3,4-tetrahydro-quinolin-3-yl]-(4-methanesulfonyl-benzyl)-amide”
“1-(1H-imidazol-5-ylmethyl)-7-pyridin-4-yl-4-[[2-(trifluoromethoxy)phenyl]carbonyl]-2,3,4,5-tetrahydro-1H-1,4-benzodiazepine”
“1-(2-chlorophenyl)-N-[6-cyano-1-[(1-methyl-1H-imidazol-5-yl)methyl]-1,2,3,4-tetrahydroquinolin-3-yl]-N-[4-(methylsulfonyl)benzyl]methanesulfonamide”
“1-methyl-1H-imidazole-4-sulfonic acid (2-bromo-allyl)-[1-(3-methyl-3H-imidazol-4-ylmethyl)-6-phenyl-1,2,3,4-tetrahydroquinolin-3-yl]-amide”
“1-methyl-1H-imidazole-4-sulfonic acid (2-bromo-allyl)-[6-cyano-1-(3H-imidazol-4-ylmethyl)-1,2,3,4-tetrahydro-quinolin-3-yl]-amide”
“1-methyl-1H-imidazole-4-sulfonic acid (4-benzenesulfonylbenzyl)-[6-cyano-1-(3-methyl-3H-imidazol-4-ylmethyl)-1,2,3,4-tetrahydro-quinolin-3-yl]-amide”
“1-methyl-1H-imidazole-4-sulfonic acid (4-methanesulfonylbenzyl)-[1-(3-methyl-3H-imidazol-4-ylmethyl)-6-phenyl-1,2,3,4-tetrahydroquinolin-3-yl]-amide”
“1-methyl-1H-imidazole-4-sulfonic acid benzyl-[6-cyano-1-(3H-imidazol-4-ylmethyl)-1,2,3,4-tetrahydro-quinolin-3-yl]-amide”
“1-methyl-1H-imidazole-4-sulfonic acid ethyl-[1-(3-methyl-3H-imidazol-4-ylmethyl)-6-phenyl-1,2,3,4-tetrahydro-quinolin-3-yl]amide”
“1-methyl-1H-imidazole-4-sulfonic acid [1-(3-methyl-3H-imidazol-4-ylmethyl)-6-phenyl-1,2,3,4-tetrahydro-quinolin-3-yl]-(2-pyrrol-1-yl-ethyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(3-methoxy-propyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(4,4-dioxo-3,4-dihydro-2H-4lambda6-benzo[1,4]oxathin-7-ylmethyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(4-ethanesulfonyl-benzyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(4-fluoro-benzyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(4-methanesulfonyl-benzyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-(5-trifluoromethyl-furan-2-ylmethyl)-amide”
“1-methyl-1H-imidazole-4-sulfonic acid [6-cyano-1-(3-methyl-3H-imidazol-4-ylmethyl)-1,2,3,4-tetrahydro-quinolin-3-yl]-[2-(2-fluoro-phenyl)-ethyl]-amide”
“1-methyl-1H-imidazole-4-sulfonic acid [6-cyano-1-(3-methyl-3H-imidazol-4-ylmethyl)-1,2,3,4-tetrahydro-quinolin-3-yl]-[2-(2-oxo-pyrrolidin-1-yl)-ethyl]-amide”
“1-methyl-1H-imidazole-4-sulfonic acid [6-cyano-1-(3-methyl-3H-imidazol-4-ylmethyl)-1,2,3,4-tetrahydro-quinolin-3-yl]-[2-(4-fluoro-phenyl)-ethyl]-amide”
“1-methyl-1H-imidazole-4-sulfonic acid [6-cyano-1-(3-methyl-3H-imidazol-4-ylmethyl)-1,2,3,4-tetrahydro-quinolin-3-yl]-[4-(1,3-dioxo-1,3-dihydro-isoindol-2-yloxy)-butyl]-amide”
“1-methyl-1H-pyrazole-4-sulfonic acid [6-cyano-1-(3-methyl-3H-imidazol-4-ylmethyl)-1,2,3,4-tetrahydro-quinolin-3-yl]-(2-fluoro-benzyl)-amide”
“1-methyl-N-(2-methylbenzyl)-N-[1-[(1-methyl-1H-imidazol-5-yl)methyl]-6-phenyl-1,2,3,4-tetrahydroquinolin-3-yl]-1H-imidazole-4-sulfonamide”
“1-methyl-N-[1-[(1-methyl-1H-imidazol-5-yl)methyl]-6-phenyl-1,2,3,4-tetrahydroquinolin-3-yl]-1H-imidazole-4-sulfonamide”
“1-methyl-N-[1-[(1-methyl-1H-imidazol-5-yl)methyl]-6-phenyl-1,2,3,4-tetrahydroquinolin-3-yl]-N-(2-methylprop-2-en-1-yl)-1H-imidazole-4-sulfonamide”
“1-methyl-N-[1-[(1-methyl-1H-imidazol-5-yl)methyl]-6-phenyl-1,2,3,4-tetrahydroquinolin-3-yl]-N-prop-2-en-1-yl-1H-imidazole-4-sulfonamide”
“1-methyl-N-[1-[(1-methyl-1H-imidazol-5-yl)methyl]-6-phenyl-1,2,3,4-tetrahydroquinolin-3-yl]-N-prop-2-yn-1-yl-1H-imidazole-4-sulfonamide”
“1-methyl-N-[1-[(1-methyl-1H-imidazol-5-yl)methyl]-6-phenyl-1,2,3,4-tetrahydroquinolin-3-yl]-N-[(5-methylisoxazol-4-yl)methyl]-1H-imidazole-4-sulfonamide”
“1-methyl-N-[1-[(1-methyl-1H-imidazol-5-yl)methyl]-6-phenyl-1,2,3,4-tetrahydroquinolin-3-yl]-N-[2-(methylsulfonyl)ethyl]-1H-imidazol-4-sulfonamide”
“1-methyl-N-[1-[(1-methyl-1H-imidazol-5-yl)methyl]-6-phenyl-1,2,3,4-tetrahydroquinolin-3-yl]-N-[[5-(trifluoromethyl)furan-2-yl]methyl]-1H-imidazole-4-sulfonamide”
“1-methylethyl 4-[([1-[(1-methyl-1H-imidazol-5-yl)methyl]-6-phenyl-1,2,3,4-tetrahydroquinolin-3-yl][(1-methyl-1H-imidazol-4-yl)sulfonyl]amino)methyl]piperidine-1-carboxylate”
“1-methylethyl 4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxylate”
“1-[(3aR,7S,8aS)-3,7-dimethyl-2-oxo-3,3a,4,7,8,8a-hexahydro-2H-cyclohepta[b]furan-6-yl]-3-(1-hydroxyethoxy)butyl acetate”
“1-[(3aR,7S,8aS)-3,7-dimethyl-2-oxo-3,3a,4,7,8,8a-hexahydro-2H-cyclohepta[b]furan-6-yl]butane-1,3-diyl diacetate”
“1-[(3aR,7S,8aS)-7-methyl-3-methylidene-2-oxo-3,3a,4,7,8,8a-hexahydro-2H-cyclohepta[b]furan-6-yl]-3-oxobutyl acetate”
“1-[(3aR,7S,8aS)-7-methyl-3-methylidene-2-oxo-3,3a,4,7,8,8a-hexahydro-2H-cyclohepta[b]furan-6-yl]butane-1,3-diyl diacetate”
10-desmethoxystreptonigrin
“18-oxa-2,5,9,11-tetraazahexacyclo[17.6.2.22,5.113,17.07,11.022,26]triaconta-1(26),7,9,13(28),14,16,19,21,22,24,26-undecaene-16-carbonitrile”
“2,2,2-trifluoroethyl 4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxylate”
2-amino-3-[2-butyl-4-(naphthalen-1-ylcarbonyl)piperazin-1-yl]propane-1-thiol
“2-methylpropyl 4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]piperidine-1-carboxylate”
“2-methylpropyl 4-[[[6-cyano-1-[(1-methyl-1H-imidazol-5-yl)methyl]-1,2,3,4-tetrahydroquinolin-3-yl](pyridin-2-ylsulfonyl)amino]methyl]piperidine-1-carboxylate”
2-oxododecanal
“2-[(2E)-3,7-dimethyl-1-(1-methyl-1H-imidazol-2-yl)octa-2,6-dien-1-yl]butanedioic acid”
“2-[(2E,6E)-3,7,11-trimethyldodeca-2,6,10-trien-1-yl]butanedioic acid”
“2-[[6-cyano-1-[(1-methyl-1H-imidazol-5-yl)methyl]-1,2,3,4-tetrahydroquinolin-3-yl](2-fluorobenzyl)amino]-2-oxoethyl acetate”
“29-oxo-18-oxa-2,6,9,11-tetraazahexacyclo[17.5.3.12,5.113,17.07,11.022,26]nonacosa-7,9,13(28), 14,16,19,21,26-octaene-16-carbonitrile”
“3,4-dihydro-2H-benzo[b][1,4]dioxepine-7-sulfonic acid [6-cyano-1-(3-methyl-3H-imidazol-4-ylmethyl)-1,2,3,4-tetrahydroquinolin-3-yl]-(4-methanesulfonyl-benzyl)-amide”
3-(1H-imidazol-2-yl)-5-methylhex-4-enoic acid
“3-(3-methyl-2-butenyl)-7,11-dimethyldodeca-2(Z),6(E), 10-triene 1-diphosphate”
3-(4-chlorophenyl)-4-cyano-1-methyl-5-[(1-methylethyl)sulfanyl]-1H-pyrrole-2-carboxylic acid
3-(4-chlorophenyl)-4-cyano-5-(cyclohexylsulfanyl)thiophene-2-carboxylic acid
3-(4-chlorophenyl)-4-cyano-5-(ethylsulfanyl)thiophene-2-carboxylic acid
3-(4-chlorophenyl)-4-cyano-5-(isopropylthio)thiophene-2-carboxylic acid
3-(4-chlorophenyl)-4-cyano-5-(methylsulfanyl)thiophene-2-carboxylic acid
3-(4-chlorophenyl)-4-cyano-5-(morpholin-4-ylsulfanyl)thiophene-2-carboxylic acid
3-(4-chlorophenyl)-4-cyano-5-[(1-methylethyl)sulfanyl]furan-2-carboxylic acid
3-(4-chlorophenyl)-4-cyano-5-[(1-methylethyl)sulfanyl]thiophene-2-carboxamide
3-(4-chlorophenyl)-4-cyano-5-[(1-methylethyl)sulfanyl]thiophene-2-carboxylic acid
3-(4-chlorophenyl)-4-cyano-5-[(1-methylpropyl)sulfanyl]thiophene-2-carboxylic acid
3-(4-chlorophenyl)-4-cyano-5-[(2-methylpropyl)sulfanyl]thiophene-2-carboxylic acid
3-(4-chlorophenyl)-4-cyano-N-cyclopropyl-5-[(1-methylethyl)sulfanyl]thiophene-2-carboxamide
3-(biphenyl-3-yl)-4-cyano-5-(propan-2-ylsulfanyl)thiophene-2-carboxylic acid
3-allylfarnesol
3-allylfarnesyl diphosphate
“3-hydroxy-1-[(3aR,7S,8aS)-7-methyl-3-methylidene-2-oxo-3,3a,4,7,8,8a-hexahydro-2H-cyclohepta[b]furan-6-yl]butyl acetate”
“3-methyl-2-[2-oxo-2-(10H-phenothiazin-10-yl)ethyl]indeno[1,2-c]pyrazol-4(2H)-one”
“3-methyl-3H-imidazole-4-sulfonic acid [6-cyano-1-(3-methyl-3H-imidazol-4-ylmethyl)-1,2,3,4-tetrahydro-quinolin-3-yl]-(4-methanesulfonyl-benzyl)-amide”
“3-methyl-thiophene-4-sulfonic acid [6-cyano-1-(3-methyl-3H-imidazol-4-ylmethyl)-1,2,3,4-tetrahydro-quinolin-3-yl]-(4-methanesulfonyl-benzyl)-amide”
“3-oxo-3-[[(2E,6E)-3,7,11-trimethyldodeca-2,6,10-trien-1-yl]oxy]propanoic acid”
3-tert-butylfarnesyl diphosphate
“3-[(tert-butylcarbamoyl-methyl)-(1-methyl-1H-imidazol-4-sulfonyl)-amino]-1-(3-methyl-3H-imidazol-4-ylmethyl)-1,2,3,4-tetrahydro-quinolin-6-carboxylic acid”
“3-[(tert-butylcarbamoyl-methyl)-(1-methyl-1H-imidazol-4-sulfonyl)-amino]-1-(3-methyl-3H-imidazol-4-ylmethyl)-1,2,3,4-tetrahydro-quinolin-6-carboxylic acid benzylamide”
“3-[(tert-butylcarbamoyl-methyl)-(1-methyl-1H-imidazol-4-sulfonyl)-amino]-1-(3-methyl-3H-imidazol-4-ylmethyl)-1,2,3,4-tetrahydro-quinolin-6-carboxylic acid tert-butylamide”
3-[3-[2-([2-[1-(tert-butoxycarbonyl)piperidin-4-yl]-1-([(4-cyanophenyl)[(1-methyl-1H-imidazol-5-yl)methyl]amino]methyl)ethyl]sulfamoyl)benzyl]phenyl]propanoic acid
“3-[[2-(tert-butylamino)-2-oxoethyl][(1-methyl-1H-imidazol-4-yl)sulfonyl]amino]-1-[(1-methyl-1H-imidazol-5-yl)methyl]-N-(1-methylpropyl)-1,2,3,4-tetrahydroquinoline-6-carboxamide”
“3-[[2-(tert-butylamino)-2-oxoethyl][(1-methyl-1H-imidazol-4-yl)sulfonyl]amino]-N,N-diethyl-1-[(1-methyl-1H-imidazol-5-yl)methyl]-1,2,3,4-tetrahydroquinoline-6-carboxamide”
“3-[[2-(tert-butylamino)-2-oxoethyl][(1-methyl-1H-imidazol-4-yl)sulfonyl]amino]-N-(1-methylethyl)-1-[(1-methyl-1H-imidazol-5-yl)methyl]-1,2,3,4-tetrahydroquinoline-6-carboxamide”
“3-[[2-(tert-butylamino)-2-oxoethyl][(1-methyl-1H-imidazol-4-yl)sulfonyl]amino]-N-cyclohexyl-1-[(1-methyl-1H-imidazol-5-yl)methyl]-1,2,3,4-tetrahydroquinoline-6-carboxamide”
“3-[[2-(tert-butylamino)-2-oxoethyl][(1-methyl-1H-imidazol-4-yl)sulfonyl]amino]-N-methyl-1-[(1-methyl-1H-imidazol-5-yl)methyl]-1,2,3,4-tetrahydroquinoline-6-carboxamide”
“4,4-(biphenyldiglyoxaldehyde)”
“4-(3-bromo-8-chloro-6,11-dihydro-5H-benzo[5,6]cyclohepta[1,2-b]pyridin-11-yl)-1-(3-cyclohexylpropanoyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-(4-oxopentanoyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-(5-oxohexanoyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-(6-oxoheptanoyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-(cyclopentylcarbonyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-(ethoxyacetyl)-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-hexanoyl-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-pentanoyl-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-pentanoyl-N-pyridin-3-ylpiperazine-2-carboxamide”
“4-(3-bromo-8-chloro-6,11-dihydro-5H-benzo[5,6]cyclohepta[1,2-b]pyridin-11-yl)-1-[(3,3-dihydroxycyclobutyl)carbonyl]-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-1-[4-(dimethylamino)-4-oxobutanoyl]-N-(pyridin-3-ylmethyl)piperazine-2-carboxamide”
“4-(3-bromo-8-chloro-6,11-dihydro-5H-benzo[5,6]cyclohepta[1,2-b]pyridin-11-yl)-N-(1-oxidopyridin-3-yl)-1-pentanoylpiperazine-2-carboxamide”
“4-(3-bromo-8-chloro-6,11-dihydro-5H-benzo[5,6]cyclohepta[1,2-b]pyridin-11-yl)-N-(1 H-imidazol-4-ylmethyl)-1-pentanoylpiperazine-2-carboxamide”
“4-(3-bromo-8-chloro-6,11-dihydro-5H-benzo[5,6]cyclohepta[1,2-b]pyridin-11-yl)-N-(2,5-dihydro-1H-imidazol-5-ylmethyl)-1-pentanoylpiperazine-2-carboxamide”
“4-(3-bromo-8-chloro-6,11-dihydro-5H-benzo[5,6]cyclohepta[1,2-b]pyridin-11-yl)-N-isoxazol-5-yl-1-pentanoylpiperazine-2-carboxamide”
“4-(3-bromo-8-chloro-6,11-dihydro-5H-benzo[5,6]cyclohepta[1,2-b]pyridin-11-yl)-N-[2-(1H-imidazol-4-yl)ethyl]-1-pentanoylpiperazine-2-carboxamide”
“4-(3-bromo-8-chloro-6,11-dihydro-5H-benzo[5,6]cyclohepta[1,2-b]pyridin-11-yl)-N1-(1-methylethyl)-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-(2-methylpropyl)-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-(cyclohexylmethyl)-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-butyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-cycloheptyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[(3,5-dimethylisoxazol-4-yl)methyl]piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[2-(1-methyl-1H-imidazol-5-yl)ethyl]piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[2-(1H-imidazol-4-yl)ethyl]piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[2-(6-methylpyridin-3-yl)ethyl]piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[3-(1H-imidazol-4-yl)propyl]piperazine-1,2-dicarboxamide”
“4-(3-bromo-8-chloro-6,11-dihydro-5H-benzo[5,6]cyclohepta[1,2-b]pyridin-11-yl)-N1-cyclohexyl-N2-[3-(2-oxopyrrolidin-1-yl)propyl]piperazine-1,2-dicarboxamide”
“4-(3-bromo-8-chloro-6,11-dihydro-5H-benzo[5,6]cyclohepta[1,2-b]pyridin-11-yl)-N1-cyclopentyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-cyclopropyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-propyl-N2-(pyridin-3-ylmethyl)piperazine-1,2-dicarboxamide”
“4-(3-bromo-8-chloro-6,11-dihydro-5H-benzo[5,6]cyclohepta[1,2-b]pyridin-11-yl)-N1-tert-butyl-N2-(pyridin-3-ylmethyl)piperazine-1,2-dicarboxamide”
4-(3-{[(4-cyano-3-naphthalen-1-yl-phenyl)-(3-methyl-3H-imidazol-4-ylmethyl)-amino]-methyl}-phenyl)-pyridine-2-carbonitrile
“4-(4-chlorophenyl)-2-[(1-methylethyl)sulfanyl]-1,3-thiazole-5-carboxylic acid”
4-(4-chlorophenyl)-5-(hydroxyacetyl)-2-[(1-methylethyl)sulfanyl]thiophene-3-carbonitrile
4-(4-chlorophenyl)-5-(methoxyacetyl)-2-[(1-methylethyl)sulfanyl]thiophene-3-carbonitrile
“4-([(2-carboxy-furan-5-sulfonyl)-[6-cyano-1-(3-methyl-3H-imidazol-4-ylmethyl)-1,2,3,4-tetrahydro-quinolin-3-yl]-amino]-methyl)-piperidine-1-carboxylic acid methyl ester”
“4-([(benzo[1,2,5]thiadiazole-4-sulfonyl)-[6-cyano-1-(3-methyl-3H-imidazol-4-ylmethyl)-1,2,3,4-tetrahydro-quinolin-3-yl]-amino]-methyl)-piperidine-1-carboxylic acid methyl ester”
“4-([[6-cyano-1-(3-methyl-3H-imidazol-4-ylmethyl)-1,2,3,4-tetrahydro-quinolin-3-yl]-cyclopropanesulfonyl-amino]-methyl)-piperidine-1-carboxylic acid methyl ester”
“4-([[6-cyano-1-(3-methyl-3H-imidazol-4-ylmethyl)-1,2,3,4-tetrahydro-quinolin-3-yl]-ethanesulfonyl-amino]-methyl)-piperidine-1-carboxylic acid methyl ester”
“4-([[6-cyano-1-(3-methyl-3H-imidazol-4-ylmethyl)-1,2,3,4-tetrahydro-quinolin-3-yl]-methanesulfonylmethanesulfonyl-amino]-methyl)-piperidine-1-carboxylic acid methyl ester”
4-cyano-3-(3-fluorophenyl)-5-(propan-2-ylsulfanyl)thiophene-2-carboxylic acid
4-cyano-3-(4-fluorophenyl)-5-(propan-2-ylsulfanyl)thiophene-2-carboxylic acid
4-cyano-3-(4-methoxyphenyl)-5-(propan-2-ylsulfanyl)thiophene-2-carboxylic acid
“4-cyano-3-(dibenzo[b,d]furan-1-yl)-5-(propan-2-ylsulfanyl)thiophene-2-carboxylic acid”
4-cyano-3-(naphthalen-2-yl)-5-(propan-2-ylsulfanyl)thiophene-2-carboxylic acid
4-cyano-3-[3-(methoxycarbonyl)phenyl]-5-(propan-2-ylsulfanyl)thiophene-2-carboxylic acid
“4-oxo-4-[[(2E,6E)-3,7,11-trimethyldodeca-2,6,10-trien-1-yl]oxy]butanoic acid”
4-[(3′-Chloro-biphenyl-3-ylmethyl)-(3-methyl-3H-imidazol-4-ylmethyl)-amino]-2-naphthalen-1-yl-benzonitrile
4-[(3-cyano-benzyl)-(3-methyl-3H-imidazol-4-ylmethyl)-amino]-2-naphthalen-1-yl-benzonitrile
4-[(3-Methyl-3H-imidazol-4-ylmethyl)-amino]-2-naphthalen-1-yl-benzonitrile
4-[(4-cyano-benzyl)-(3-methyl-3H-imidazol-4-ylmethyl)-amino]-2-naphthalen-1-yl-benzonitrile
“4-[(5-[[4-(10,11-dihydro-5H-dibenzo[a,d][7]annulen-5-yl)azepan-1-yl]methyl]-1H-imidazol-1-yl)methyl]benzonitrile”
“4-[(5-[[4-(10,11-dihydro-5H-dibenzo[a,d][7]annulen-5-yl)piperidin-1-yl]methyl]-1H-imidazol-1-yl)methyl]benzonitrile”
“4-[(5-[[4-(5-oxido-7-propyl-7,12-dihydrodibenzo[c,f][1,2,8]oxathiazocin-12-yl)azepan-1-yl]methyl]-1H-imidazol-1-yl)methyl]benzonitrile”
“4-[(5-[[4-(7-butyl-5-oxido-7,12-dihydrodibenzo[c,f][1,2,8]oxathiazocin-12-yl)azepan-1-yl]methyl]-1H-imidazol-1-yl)methyl]benzonitrile”
“4-[(5-[[4-(7-methyl-5-oxido-7,12-dihydrodibenzo[c,f][1,2,8]oxathiazocin-12-yl)azepan-1-yl]methyl]-1H-imidazol-1-yl)methyl]benzonitrile”
“4-[(5-[[4-(7-methyl-5-oxido-7,12-dihydrodibenzo[c,f][1,2,8]oxathiazocin-12-yl)piperidin-1-yl]methyl]-1H-imidazol-1-yl)methyl]benzonitrile”
“4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]benzoic acid”
4-[Biphenyl-3-ylmethyl-(3-methyl-3H-imidazol-4-ylmethyl)-amino]-2-naphthalen-1-yl-benzonitrile
4-[Hexyl-(3-methyl-3H-imidazol-4-ylmethyl)-amino]-2-naphthalen-1-yl-benzonitrile
4-[Methyl-(3-methyl-3H-imidazol-4-ylmethyl)-amino]-2-naphthalen-1-yl-benzonitrile
“4-[N,N-bis((1H-imidazol-4-yl)methyl)aminomethyl]-2-(1-(o-tolyl))benzoylmethionine trifluoroacetate”
4-[N-(1-H-imidazol-4-yl)methylamino]-2-(2-methoxyphenyl)-benzoylmethionine
4-[N-(1-H-imidazol-4-yl)methylamino]-2-(2-methylphenyl)-benzoylmethionine
4-[N-(1H-imidazol-4-yl)methylamino]-2-phenylbenzoylmethionine
“4-[[(6-cyano-1-pyridin-3-ylmethyl-1,2,3,4-tetrahydro-quinolin-3-yl)-(pyridine-2-sulfonyl)-amino]-methyl]-piperidine-1-carboxylic acid methyl ester”
“4-[[5-([4-[7-(3-morpholin-4-ylpropyl)-5-oxido-7,12-dihydrodibenzo[c,f][1,2,8]oxathiazocin-12-yl]azepan-1-yl]methyl)-1H-imidazol-1-yl]methyl]benzonitrile”
“4-[[[6-cyano-1-(3-methyl-3H-imidazol-4-ylmethyl)-1,2,3,4-tetrahydro-quinolin-3-yl]-(1,5-dimethyl-1H-imidazole-4-sulfonyl)-amino]-methyl]-piperidine-1-carboxylic acid methyl ester”
“4-[[[6-cyano-1-(3-methyl-3H-imidazol-4-ylmethyl)-1,2,3,4-tetrahydro-quinolin-3-yl]-(1,5-dimethyl-1H-imidazole-4-sulfonyl)-amino]-methyl]-piperidine-1-carboxylic acid tert-butyl ester”
“4-[[[6-cyano-1-(3-methyl-3H-imidazol-4-ylmethyl)-1,2,3,4-tetrahydro-quinolin-3-yl]-(2-oxo-2H-chromene-6-sulfonyl)-amino]-methyl]-piperidine-1-carboxylic acid methyl ester”
“4-[[[6-cyano-1-(3-methyl-3H-imidazol-4-ylmethyl)-1,2,3,4-tetrahydro-quinolin-3-yl]-(3,4-dihydro-2H-benzo[b][1,4]dioxepine-7-sulfonyl)-amino]-methyl]-piperidine-1-carboxylic acid methyl ester”
“4-[[[6-cyano-1-(3-methyl-3H-imidazol-4-ylmethyl)-1,2,3,4-tetrahydro-quinolin-3-yl]-(3-methyl-3H-imidazole-4-sulfonyl)-amino]-methyl]-piperidine-1-carboxylic acid methyl ester”
“4-[[[6-cyano-1-(3-methyl-3H-imidazol-4-ylmethyl)-1,2,3,4-tetrahydro-quinolin-3-yl]-(pyridine-2-sulfonyl)-amino]-methyl]-piperidine-1-carboxylic acid ethylamide”
“4-[[[6-cyano-1-(3-methyl-3H-imidazol-4-ylmethyl)-1,2,3,4-tetrahydro-quinolin-3-yl]-(pyridine-2-sulfonyl)-amino]-methyl]-piperidine-1-carboxylic acid isobutyl ester”
“4-[[[6-cyano-1-(3-methyl-3H-imidazol-4-ylmethyl)-1,2,3,4-tetrahydro-quinolin-3-yl]-(pyridine-2-sulfonyl)-amino]-methyl]-piperidine-1-carboxylic acid methyl ester”
“4-[[[6-cyano-1-[(1-methyl-1H-imidazol-5-yl)methyl]-1,2,3,4,6,7-hexahydroquinolin-3-yl](pyridin-2-ylsulfonyl)amino]methyl]-N,N-dimethylpiperidine-1-carboxamide”
“4-[[[6-cyano-1-[(1-methyl-1H-imidazol-5-yl)methyl]-1,2,3,4,6,7-hexahydroquinolin-3-yl](pyridin-2-ylsulfonyl)amino]methyl]-N-(1-methylethyl)piperidine-1-carboxamide”
“4-[[[6-cyano-1-[(1-methyl-1H-imidazol-5-yl)methyl]-1,2,3,4-tetrahydroquinolin-3-yl](pyridin-2-ylsulfonyl)amino]methyl]-N-ethylpiperidine-1-carboxamide”
“5,7-dihydroxy-2-[(5R)-1-hydroxy-5-[[(3S,8aS)-3-hydroxy-5,5,8a-trimethyl-2-methylidenedecahydronaphthalen-1-yl]methyl]-2-methoxy-6-methyl-4-oxo-1,5,8,8a-tetrahydronaphthalen-4a(4H)-yl]-4H-chromen-4-one”
“5,7-dihydroxy-2-[(5R)-1-hydroxy-5-[[(3S,8aS)-3-hydroxy-5,5,8a-trimethyl-2-methylidenedecahydronaphthalen-1-yl]methyl]-6-methyl-2-oxo-1,5,8,8a-tetrahydronaphthalen-4a(2H)-yl]-4H-chromen-4-one”
“5,9,13-trimethyl-8,12-tetradecadiene-2,3-dione”
“5,9-dimethyl-8-decene-2,3-dione”
5-(butylsulfanyl)-3-(4-chlorophenyl)-4-cyanothiophene-2-carboxylic acid
5-acetyl-4-(4-chlorophenyl)-2-[(1-methylethyl)sulfanyl]thiophene-3-carbonitrile
5-methyl-3-(1-methyl-1H-imidazol-2-yl)hex-4-enoic acid
“5-oxo-5-[[(2E,6E)-3,7,11-trimethyldodeca-2,6,10-trien-1-yl]oxy]pentanoic acid”
5-[(3-cyano-benzyl)-(3-methyl-3H-imidazol-4-ylmethyl)-amino]-2′-methyl-biphenyl-2-carbonitrile
5-[(4-cyano-benzyl)-(3-methyl-3H-imidazol-4-ylmethyl)-amino]-2′-methyl-biphenyl-2-carbonitrile
“5-[4-(3-bromo-8-chloro-6,11-dihydro-5H-benzo[5,6]cyclohepta[1,2-b]pyridin-11-yl)-2-[(pyridin-3-ylmethyl)carbamoyl]piperazin-1-yl]-5-oxopentyl acetate”
“6-([6-cyano-1-[(1-methyl-1H-imidazol-5-yl)methyl]-1,2,3,4-tetrahydroquinolin-3-yl][(1-methyl-1H-imidazol-4-yl)sulfonyl]amino)hexanamide”
“6-[(4-hydroxyphenyl)(1H-imidazol-1-yl)methyl]-4-phenyl-1,2-dihydroquinolin-2-ol”
“6-[[6-cyano-1-[(1-methyl-1H-imidazol-5-yl)methyl]-1,2,3,4-tetrahydroquinolin-3-yl](pyridin-2-ylsulfonyl)amino]hexanamide”
“6-[[[6-cyano-1-(3-methyl-3H-imidazol-4-ylmethyl)-1,2,3,4-tetrahydro-quinolin-3-yl]-(pyridine-2-sulfonyl)-amino]-methyl]-pyridine-2-carboxylic acid methyl ester”
“6-[[[6-cyano-1-[(1-methyl-1H-imidazol-5-yl)methyl]-1,2,3,4-tetrahydroquinolin-3-yl](pyridin-2-ylsulfonyl)amino]methyl]pyridine-2-carboxylic acid”
ABT-100
ABT-839
acetylgliotoxin
acetylshikonin
actinoplanic acid A
actinoplanic acid B
alphabeta-dehydrocurvularin
andrastin A
andrastin B
andrastin C
asukamycin
AZD3409
barceloneic acid A
“benzyl 4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxylate”
Biphenyl-3-carboxylic acid (4-cyano-3-naphthalen-1-yl-phenyl)-(3-methyl-3H-imidazol-4-ylmethyl)-amide
Biphenyl-3-sulfonic acid (4-cyano-3-naphthalen-1-yl-phenyl)-(3-methyl-3H-imidazol-4-ylmethyl)-amide
BMS-214662
BMS-339941
BMS-388891
chaetomellic acid A
chaetomellic acid B
citreohybridone A
citreohybridone B
clavaric acid
“CP-225,917”
“CP-263,114”
CVFM
CVIM
cylindrol A
cylindrol A1
Cys-Val-Phe-aminohexanoate
Cys-Val-Phe-Cys
Cys-Val-Phe-Met
Cys-Val-Phe-Phe
dehydrascorbic acid 6-palmitate
deoxyshikonin
desloratadine
“di-tert-butyl 2-[(2E,6E,10E)-1,3,7-trimethyl-1-(1-methyl-1H-imidazol-2-yl)dodeca-2,6,10-trien-1-yl]butanedioate”
DPI-1
econazole
ethyl 3-(4-chlorophenyl)-4-cyano-5-[(1-methylethyl)sulfanyl]thiophene-2-carboxylate
“ethyl 4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]piperidine-1-carboxylate”
“ethyl 4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxylate”
“ethyl 4-[[[6-cyano-1-[(1-methyl-1H-imidazol-5-yl)methyl]-1,2,3,4-tetrahydroquinolin-3-yl](pyridin-2-ylsulfonyl)amino]methyl]piperidine-1-carboxylate”
farnesyltransferase inhibitors I and II
FTI-2148
FTI-2153
FTI-276
FTI-277
fusidienol
fusidienol A
GGTI-297
GGTI-298
gliotoxin
granaticin A
granaticin B
kalafungin
kampanol A
kampanol B
KKSKTKCVIM
KKTKSKCVIM
KTSCVAM
KTSCVFM
KTSCVIA
KTSCVIF
KTSSVIM
kurasoin A
kurasoin B
“L-739,749”
“L-739,750”
“L-739,750”
“L-744,832”
“L-745,631”
“L-778,123”
“L-788,123”
L-Cys-L-Val-L-Leu-L-Ser
L-penicillamine-Ile-Ile-Met
L-penicillamine-Val-Ile-Ala
L-penicillamine-Val-Ile-aminohexanoate
L-penicillamine-Val-Ile-Cys
L-penicillamine-Val-Ile-Gln
L-penicillamine-Val-Ile-homoserine
L-penicillamine-Val-Ile-Met
L-penicillamine-Val-Ile-Phe
L-penicillamine-Val-Val-Met
L778123
loratadine
Lys-Lys-Cys-Val-Ile-Met
manumycin
manumycin A
“methyl 2-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]pyridine-4-carboxylate”
“methyl 4-[([(2-chlorophenyl)sulfonyl][6-cyano-1-[(1-methyl-1H-imidazol-5-yl)methyl]-1,2,3,4-tetrahydroquinolin-3-yl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(1,2-dimethyl-1H-imidazol-4-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(1-methyl-1H-imidazol-2-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]benzoate”
“methyl 4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(1-methyl-1H-pyrazol-4-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(3-fluorophenyl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(3-methylthiophen-2-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(4-methoxyphenyl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][(4-methylthiophen-2-yl)sulfonyl]amino)methyl]piperidine-1-carboxylate”
“methyl 4-[([6-cyano-1-[(1-methyl-1H-imidazol-5-yl)methyl]-1,2,3,4-tetrahydroquinolin-3-yl][[3-(methoxycarbonyl)thiophen-2-yl]sulfonyl]amino)methyl]piperidine-1-carboxylate”
“methyl 4-[([6-cyano-1-[(1-methyl-1H-imidazol-5-yl)methyl]-2-oxo-1,2,3,4-tetrahydroquinolin-3-yl][(1-methyl-1H-imidazol-4-yl)sulfonyl]amino)methyl]piperidine-1-carboxylate”
“methyl 4-[([[4-(acetylamino)phenyl]sulfonyl][6-cyano-1-[(1-methyl-1H-imidazol-5-yl)methyl]-1,2,3,4-tetrahydroquinolin-3-yl]amino)methyl]piperidine-1-carboxylate”
“methyl 4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](dimethylsulfamoyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](methylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](naphthalen-1-ylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](phenylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](propylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](pyridin-3-ylsulfonyl)amino]methyl]piperidine-1-carboxylate”
“methyl 4-[[[6-cyano-1-[(1-methyl-1H-imidazol-5-yl)methyl]-1,2,3,4-tetrahydroquinolin-3-yl](quinolin-8-ylsulfonyl)amino]methyl]piperidine-1-carboxylate”
“methyl 4-[[[6-cyano-1-[1-(1H-imidazol-5-yl)ethyl]-1,2,3,4-tetrahydroquinolin-3-yl](pyridin-2-ylsulfonyl)amino]methyl]piperidine-1-carboxylate”
methyl N-(1-cysteinylpiperidin-4-yl)-N-(phenylcarbonyl)-L-methioninate
methyl N-(1-[[1-(4-cyanobenzyl)-1H-imidazol-4-yl]methyl]piperidin-4-yl)-N-(phenylcarbonyl)-L-isoleucinate
methyl N-(1-[[1-(4-cyanobenzyl)-1H-imidazol-4-yl]methyl]piperidin-4-yl)-N-(phenylcarbonyl)-L-methioninate
methyl N-(1-[[1-(4-cyanobenzyl)-1H-imidazol-4-yl]methyl]piperidin-4-yl)-N-(phenylcarbonyl)-L-phenylalaninate
methyl N-(1-[[1-(4-cyanobenzyl)-1H-imidazol-5-yl]methyl]piperidin-4-yl)-N-(phenylcarbonyl)-L-phenylalaninate
methyl N-(phenylcarbonyl)-N-{1-([1-[4-(trifluoromethyl)benzyl]-1H-imidazol-4-yl]methyl)piperidin-4-yl}-L-methioninate
“methyl N-([2-[(1E,6E,10E)-4,4-bis(ethoxycarbonyl)-7,11,15-trimethylhexadeca-1,6,10,14-tetraen-1-yl]-1-methyl-1H-imidazol-5-yl]methyl)-L-valyl-L-phenylalanyl-L-methioninate”
“methyl N-([2-[(6E,10E)-4,4-bis(ethoxycarbonyl)-7,11,15-trimethylhexadeca-6,10,14-trien-1-yl]-1-methyl-1H-imidazol-5-yl]methyl)-L-valyl-L-phenylalanyl-L-methioninate”
methyl N-([2-[4-ethoxy-2-(ethoxycarbonyl)-4-oxobutyl]-1-methyl-1H-imidazol-5-yl]methyl)-L-valyl-L-phenylalanyl-L-methioninate
methyl N-([2-[4-ethoxy-2-(ethoxycarbonyl)-4-oxobutyl]-1-methyl-1H-imidazol-5-yl]methyl)-L-valyl-Lisoleucyl-L-alaninate
methyl N-([2-[5-ethoxy-3-(methoxycarbonyl)-5-oxopentyl]-1-methyl-1H-imidazol-5-yl]methyl)-L-valyl-L-isoleucyl-L-alaninate
methyl N-([2-[5-ethoxy-3-(methoxycarbonyl)-5-oxopentyl]-1-methyl-1H-imidazol-5-yl]methyl)-L-valyl-L-phenylalanyl-L-methioninate
methyl N-([2-[6-ethoxy-4-(ethoxycarbonyl)-6-oxohexyl]-1-methyl-1H-imidazol-5-yl]methyl)-L-valyl-L-isoleucyl-L-alaninate
methyl N-([2-[6-ethoxy-4-(ethoxycarbonyl)-6-oxohexyl]-1-methyl-1H-imidazol-5-yl]methyl)-L-valyl-L-phenylalanyl-L-methioninate
“methyl N-([3-[methyl(1-methylidene-2,3-disulfanylpropyl)amino]-5-phenyl-2,3-dihydro-1H-1,4-benzodiazepin-1-yl]acetyl)methioninate”
methyl N-benzyl-N-(1-[[1-(4-cyanobenzyl)-1H-imidazol-4-yl]methyl]piperidin-4-yl)-L-methioninate
methyl N-[(6E)-2-benzyl-5-(1-methylethyl)-8-(sulfanylmethyl)dec-6-enoyl]methioninate
methyl N-[1-[(1-benzyl-1H-imidazol-4-yl)methyl]piperidin-4-yl]-N-(phenylcarbonyl)-L-methioninate
“methyl [2-[[6-cyano-1-[(1-methyl-1H-imidazol-5-yl)methyl]-1,2,3,4-tetrahydroquinolin-3-yl](pyridin-2-ylsulfonyl)amino]ethyl]carbamate”
“methyl [3-([6-cyano-1-[(1-methyl-1H-imidazol-5-yl)methyl]-1,2,3,4-tetrahydroquinolin-3-yl][(1-methyl-1H-imidazol-4-yl)sulfonyl]amino)propyl]carbamate”
“methyl [3-[[6-cyano-1-[(1-methyl-1H-imidazol-5-yl)methyl]-1,2,3,4-tetrahydroquinolin-3-yl](pyridin-2-ylsulfonyl)amino]propyl]carbamate”
“methyl [4-[[6-cyano-1-[(1-methyl-1H-imidazol-5-yl)methyl]-1,2,3,4-tetrahydroquinolin-3-yl](pyridin-2-ylsulfonyl)amino]butyl]carbamate”
“methyl [4-[[6-cyano-1-[(1-methyl-1H-imidazol-5-yl)methyl]-1,2,3,4-tetrahydroquinolin-3-yl](pyridin-2-ylsulfonyl)amino]butyl]ethylcarbamate”
“methyl [4-[[6-cyano-1-[(1-methyl-1H-imidazol-5-yl)methyl]-1,2,3,4-tetrahydroquinolin-3-yl](pyridin-2-ylsulfonyl)amino]butyl]methylcarbamate”
“methyl [6-cyano-1-[(1-methyl-1H-imidazol-5-yl)methyl]-1,2,3,4-tetrahydroquinolin-3-yl](2-fluorobenzyl)carbamate”
N′-[(1Z)-1-(3-hydroxy-1-oxo-1H-inden-2-yl)ethylidene]-3-(10H-phenothiazin-10-yl)propanehydrazide
“N,1-dimethyl-N-[1-[(1-methyl-1H-imidazol-5-yl)methyl]-6-phenyl-1,2,3,4-tetrahydroquinolin-3-yl]-1H-imidazole-4-sulfonamide”
N-(1-benzyl-2-[(4-cyanophenyl)[(1-methyl-1H-imidazol-5-yl)methyl]amino]ethyl)-1-methyl-1H-imidazole-4-sulfonamide
N-(1-benzyl-2-[(4-cyanophenyl)[(1-methyl-1H-imidazol-5-yl)methyl]amino]ethyl)-2-methylbenzenesulfonamide
“N-(1-bromoethenyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-(2,1,3-benzothiadiazol-4-ylmethyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-(2,1,3-benzothiadiazol-4-ylmethyl)-N-[6-cyano-1-[(1-methyl-1H-imidazol-5-yl)methyl]-1,2,3,4-tetrahydroquinolin-3-yl]pyridine-2-sulfonamide”
“N-(2,1,3-benzoxadiazol-5-ylmethyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-(2-aminoethyl)-4-([6-cyano-1-[(1-methyl-1H-imidazol-5-yl)methyl]-1,2,3,4-tetrahydroquinolin-3-yl][(1-methyl-1H-imidazol-4-yl)sulfonyl]amino)butanamide”
“N-(2-aminoethyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-(2-aminoethyl)-N-[6-cyano-1-[(1-methyl-1H-imidazol-5-yl)methyl]-1,2,3,4-tetrahydroquinolin-3-yl]pyridine-2-sulfonamide”
“N-(2-chloroprop-2-en-1-yl)-1-methyl-N-[1-[(1-methyl-1H-imidazol-5-yl)methyl]-6-phenyl-1,2,3,4-tetrahydroquinolin-3-yl]-1H-imidazole-4-sulfonamide”
“N-(2-fluorobenzyl)-1-methyl-N-[1-[(1-methyl-1H-imidazol-5-yl)methyl]-6-phenyl-1,2,3,4-tetrahydroquinolin-3-yl]-1H-imidazole-4-sulfonamide”
“N-(3-aminopropyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-(4-bromobenzyl)-N-[6-cyano-1-[(1-methyl-1H-imidazol-5-yl)methyl]-1,2,3,4-tetrahydroquinolin-3-yl]pyridine-2-sulfonamide”
“N-(4-bromophenyl)-4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxamide”
“N-(4-cyanobenzyl)-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-([1-[(4-chlorophenyl)carbonyl]piperidin-4-yl]methyl)-N-[6-cyano-1-[(1-methyl-1H-imidazol-5-yl)methyl]-1,2,3,4,6,7-hexahydroquinolin-3-yl]pyridine-2-sulfonamide”
“N-([2-[(1E,6E,10E)-4-carboxy-4-(ethoxycarbonyl)-7,11,15-trimethylhexadeca-1,6,10,14-tetraen-1-yl]-1-methyl-1H-imidazol-5-yl]methyl)-L-valyl-L-phenylalanyl-L-methionine”
“N-([2-[(6E,10E)-4-carboxy-4-(ethoxycarbonyl)-7,11,15-trimethylhexadeca-6,10,14-trien-1-yl]-1-methyl-1H-imidazol-5-yl]methyl)-L-valyl-L-phenylalanyl-L-methionine”
“N-([2-[(6E,10E)-4-carboxy-4-(ethoxycarbonyl)-7,11,15-trimethylhexadeca-6,10,14-trien-1-yl]-1-methyl-1H-imidazol-5-yl]methyl)-L-valyl-L-phenylalanyl-L-methionine”
“N-([5-[(2R)-2,3-dihydro-1,4-benzodioxin-2-ylmethoxy]-2′-methoxybiphenyl-2-yl]carbonyl)-D-methionine”
“N-([5-[(2R)-2,3-dihydro-1,4-benzodioxin-2-ylmethoxy]-2′-methoxybiphenyl-2-yl]carbonyl)-L-methionine”
“N-([5-[(2R)-2,3-dihydro-1,4-benzodioxin-2-ylmethoxy]-2′-methylbiphenyl-2-yl]carbonyl)-D-methionine”
“N-([5-[(2R)-2,3-dihydro-1,4-benzodioxin-2-ylmethoxy]-2′-methylbiphenyl-2-yl]carbonyl)-L-methionine”
“N-([5-[(2S)-2,3-dihydro-1,4-benzodioxin-2-ylmethoxy]-2′-methoxybiphenyl-2-yl]carbonyl)-D-methionine”
“N-([5-[(2S)-2,3-dihydro-1,4-benzodioxin-2-ylmethoxy]-2′-methoxybiphenyl-2-yl]carbonyl)-L-methionine”
“N-([5-[(2S)-2,3-dihydro-1,4-benzodioxin-2-ylmethoxy]-2′-methylbiphenyl-2-yl]carbonyl)-D-methionine”
“N-([5-[(2S)-2,3-dihydro-1,4-benzodioxin-2-ylmethoxy]-2′-methylbiphenyl-2-yl]carbonyl)-L-methionine”
“N-benzyl-1-methyl-N-[1-[(1-methyl-1H-imidazol-5-yl)methyl]-6-phenyl-1,2,3,4-tetrahydroquinolin-3-yl]-1H-imidazole-4-sulfonamide”
“N-benzyl-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-butyl-N-[6-cyano-1-[(1-methyl-1H-imidazol-5-yl)methyl]-1,2,3,4-tetrahydroquinolin-3-yl]pyridine-2-sulfonamide”
“N-tert-butyl-2-[(1-methyl-1H-imidazole-4-sulfonyl)-[1-(3-methyl-3H-imidazol-4-ylmethyl)-6-phenyl-1,2,3,4-tetrahydro-quinolin-3-yl]-amino]-acetamide”
“N-tert-butyl-4-[[[1-[(1-methyl-1H-imidazol-5-yl)methyl]-6-phenyl-1,2,3,4-tetrahydroquinolin-3-yl](pyridin-2-ylsulfonyl)amino]methyl]piperidine-1-carboxamide”
“N-tert-butyl-4-[[[6-cyano-1-[(1-methyl-1H-imidazol-5-yl)methyl]-1,2,3,4,6,7-hexahydroquinolin-3-yl](pyridin-2-ylsulfonyl)amino]methyl]piperidine-1-carboxamide”
“N-tert-butyl-N2-[1-[(1-methyl-1H-imidazol-5-yl)methyl]-6-phenyl-1,2,3,4-tetrahydroquinolin-3-yl]-N2-(methylsulfonyl)glycinamide”
“N-tert-butyl-N2-[1-[(1-methyl-1H-imidazol-5-yl)methyl]-6-phenyl-1,2,3,4-tetrahydroquinolin-3-yl]-N2-(phenylsulfonyl)glycinamide”
“N-tert-butyl-N2-[1-[(1-methyl-1H-imidazol-5-yl)methyl]-6-phenyl-1,2,3,4-tetrahydroquinolin-3-yl]-N2-(pyridin-2-ylsulfonyl)glycinamide”
“N-tert-butyl-N2-[1-[(1-methyl-1H-imidazol-5-yl)methyl]-6-phenyl-1,2,3,4-tetrahydroquinolin-3-yl]-N2-(thiophen-2-ylsulfonyl)glycinamide”
“N-tert-butyl-N2-[6-cyano-1-(1-methyl-1H-imidazol-5-yl)-1,2,3,4-tetrahydroquinolin-3-yl]-N2-[(1-methyl-1H-imidazol-2-yl)sulfonyl]glycinamide”
“N-tert-butyl-N2-[6-cyano-1-(1H-imidazol-4-yl)-1,2,3,4-tetrahydroquinolin-3-yl]-N2-[(1-methyl-1H-imidazol-2-yl)sulfonyl]glycinamide”
“N-tert-butyl-N2-[6-cyano-1-(2,4-dimethyl-1-1,3-thiazol-5-yl)-1,2,3,4-tetrahydroquinolin-3-yl]-N2-[(1-methyl-1H-imidazol-2-yl)sulfonyl]glycinamide”
“N-tert-butyl-N2-[6-cyano-1-(4-fluorophenyl)-1,2,3,4-tetrahydroquinolin-3-yl]-N2-[(1-methyl-1H-imidazol-2-yl)sulfonyl]glycinamide”
“N-tert-butyl-N2-[6-cyano-1-(5-methylisoxazol-4-yl)-1,2,3,4-tetrahydroquinolin-3-yl]-N2-[(1-methyl-1H-imidazol-2-yl)sulfonyl]glycinamide”
“N-tert-butyl-N2-[6-cyano-1-[(1-methyl-1H-imidazol-5-yl)methyl]-1,2,3,4-tetrahydroquinolin-3-yl]-N2-(pyridin-2-ylsulfonyl)glycinamide”
“N-tert-butyl-N2-[6-cyano-1-[(1-methyl-1H-imidazol-5-yl)methyl]-1,2,3,4-tetrahydroquinolin-3-yl]-N2-(pyridin-3-ylsulfonyl)glycinamide”
“N-tert-butyl-N2-[6-cyano-1-[(1-methyl-1H-imidazol-5-yl)methyl]-1,2,3,4-tetrahydroquinolin-3-yl]-N2-(pyridin-4-ylsulfonyl)glycinamide”
“N-tert-butyl-N2-[6-cyano-1-[(1-methyl-1H-imidazol-5-yl)methyl]-1,2,3,4-tetrahydroquinolin-3-yl]-N2-[(1-methyl-1H-imidazol-2-yl)sulfonyl]glycinamide”
“N-tert-butyl-N2-[6-cyano-1-[(1-methyl-1H-imidazol-5-yl)methyl]-1,2,3,4-tetrahydroquinolin-3-yl]-N2-[(4-fluorophenyl)sulfonyl]glycinamide”
“N-tert-butyl-N2-[6-cyano-1-[2-(1,3-dioxo-1,3-dihydro-2H-isoindol-2-yl)ethyl]-1,2,3,4-tetrahydroquinolin-3-yl]-N2-[(1-methyl-1H-imidazol-2-yl)sulfonyl]glycinamide”
“N-[(1-acetylpiperidin-4-yl)methyl]-N-[6-cyano-1-[(1-methyl-1H-imidazol-5-yl)methyl]-1,2,3,4,6,7-hexahydroquinolin-3-yl]pyridine-2-sulfonamide”
“N-[(1-acetylpiperidin-4-yl)methyl]-N-[6-cyano-1-[(1-methyl-1H-imidazol-5-yl)methyl]-1,2,3,4-tetrahydroquinolin-3-yl]pyridine-2-sulfonamide”
“N-[(1-bromonaphthalen-2-yl)methyl]-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-[(1-bromonaphthalen-2-yl)methyl]-N-[6-cyano-1-[(1-methyl-1H-imidazol-5-yl)methyl]-1,2,3,4-tetrahydroquinolin-3-yl]pyridine-2-sulfonamide”
“N-[(1-butanoylpiperidine-4-yl)methyl]-N-[6-cyano-1-[(1-methyl-1H-imidazol-5-yl)methyl]-1,2,3,4,6,7-hexahydroquinolin-3-yl]pyridine-2-sulfonamide”
“N-[(1-butanoylpiperidin-4-yl)methyl]-N-[6-cyano-1-[(1-methyl-1H-imidazol-5-yl)methyl]-1,2,3,4-tetrahydroquinolin-3-yl]pyridine-2-sulfonamide”
“N-[(2E)-but-2-en-1-yl]-1-methyl-N-[1-[(1-methyl-1H-imidazol-5-yl)methyl]-6-phenyl-1,2,3,4-tetrahydroquinolin-3-yl]-1H-imidazole-4-sulfonamide”
“N-[(3-bromonaphthalen-2-yl)methyl]-N-[6-cyano-1-[(1-methyl-1H-imidazol-5-yl)methyl]-1,2,3,4-tetrahydroquinolin-3-yl]pyridine-2-sulfonamide”
“N-[(5-[[(2R)-2,3-dihydro-1,4-benzodioxin-2-ylmethyl]carbamoyl]-2′-methoxybiphenyl-2-yl)carbonyl]-D-methionine”
“N-[(5-[[(2R)-2,3-dihydro-1,4-benzodioxin-2-ylmethyl]carbamoyl]-2′-methoxybiphenyl-2-yl)carbonyl]-L-methionine”
“N-[(5-[[(2R)-2,3-dihydro-1,4-benzodioxin-2-ylmethyl]carbamoyl]-2′-methylbiphenyl-2-yl)carbonyl]-D-methionine”
“N-[(5-[[(2R)-2,3-dihydro-1,4-benzodioxin-2-ylmethyl]carbamoyl]-2′-methylbiphenyl-2-yl)carbonyl]-L-methionine”
“N-[(5-[[(2S)-2,3-dihydro-1,4-benzodioxin-2-ylmethyl]carbamoyl]-2′-methoxybiphenyl-2-yl)carbonyl]-D-methionine”
“N-[(5-[[(2S)-2,3-dihydro-1,4-benzodioxin-2-ylmethyl]carbamoyl]-2′-methoxybiphenyl-2-yl)carbonyl]-L-methionine”
“N-[(5-[[(2S)-2,3-dihydro-1,4-benzodioxin-2-ylmethyl]carbamoyl]-2′-methylbiphenyl-2-yl)carbonyl]-D-methionine”
“N-[(5-[[(2S)-2,3-dihydro-1,4-benzodioxin-2-ylmethyl]carbamoyl]-2′-methylbiphenyl-2-yl)carbonyl]-L-methionine”
“N-[(6-chloro-2,1,3-benzothiadiazol-5-yl)methyl]-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-[(6-chloro-2,1,3-benzothiadiazol-5-yl)methyl]-N-[6-cyano-1-[(1-methyl-1H-imidazol-5-yl)methyl]-1,2,3,4-tetrahydroquinolin-3-yl]pyridine-2-sulfonamide” N-[(6E)-2-benzyl-5-(1-methylethyl)-8-(sulfanylmethyl)dec-6-enoyl]methionine
“N-[1-(tert-butylamino)ethenyl]-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-[1-[(1-methyl-1H-imidazol-5-yl)methyl]-6-phenyl-1,2,3,4-tetrahydroquinolin-3-yl]pyridine-2-sulfonamide”
N-[2-([2-[(2-amino-3-sulfanylpropyl)amino]-3-methylbutyl]amino)-3-phenylpropyl]methionine
N-[2-([[1-(4-cyanobenzyl)-1H-imidazol-5-yl]acetyl]amino)-3-methylpentyl]-N-(naphthalen-1-ylmethyl)glycylmethionine
“N-[2-chloro-4-(methylsulfonyl)benzyl]-N-[6-cyano-1-[(1-methyl-1H-imidazol-5-yl)methyl]-1,2,3,4-tetrahydroquinolin-3-yl]-1-methyl-1H-imidazole-4-sulfonamide”
“N-[4-[[6-cyano-1-[(1-methyl-1H-imidazol-5-yl)methyl]-1,2,3,4-tetrahydroquinolin-3-yl](2-fluorobenzyl)sulfamoyl]phenyl]acetamide”
“N-[6-bromo-1-[(1-methyl-1H-imidazol-5-yl)methyl]-2-oxo-1,2,3,4-tetrahydroquinolin-3-yl]-1-methyl-1H-imidazole-4-sulfonamide”
“N-[6-bromo-1-[(1-methyl-1H-imidazol-5-yl)methyl]-2-oxo-1,2,3,4-tetrahydroquinolin-3-yl]-1-methyl-1H-imidazole-4-sulfonamide”
“N-[6-cyano-1-(1,2,3-thiadiazol-5-ylmethyl)-1,2,3,4-tetrahydroquinolin-3-yl]-N-(2-fluorobenzyl)-1-methyl-1H-imidazole-4-carboxamide”
“N-[6-cyano-1-(1H-imidazol-5-ylmethyl)-1,2,3,4-tetrahydroquinolin-3-yl]-N-(2-fluorobenzyl)-1-methyl-1H-imidazole-4-carboxamide”
“N-[6-cyano-1-(3-methyl-3H-imidazol-4-ylmethyl)-1,2,3,4-tetrahydro-quinolin-3-yl]-2-dimethylamino-N-(2-fluoro-benzyl)-acetamide”
“N-[6-cyano-1-(3-methyl-3H-imidazol-4-ylmethyl)-1,2,3,4-tetrahydro-quinolin-3-yl]-N-(2-fluoro-benzyl)-3-methylsulfanylpropionamide”
“N-[6-cyano-1-(3-methyl-3H-imidazol-4-ylmethyl)-1,2,3,4-tetrahydro-quinolin-3-yl]-N-(2-fluoro-benzyl)-4-methoxy-benzenesulfonamide”
“N-[6-cyano-1-(3-methyl-3H-imidazol-4-ylmethyl)-1,2,3,4-tetrahydro-quinolin-3-yl]-N-(4-methanesulfonyl-benzyl)-benzenesulfonamide”
“N-[6-cyano-1-[(1-methyl-1H-imidazol-5-yl)methyl]-1,2,3,4,6,7-hexahydroquinolin-3-yl]-N-([1-[(1-methyl-1H-imidazol-4-yl)sulfonyl]piperidin-4-yl]methyl)pyridine-2-sulfonamide”
“N-[6-cyano-1-[(1-methyl-1H-imidazol-5-yl)methyl]-1,2,3,4,6,7-hexahydroquinolin-3-yl]-N-[(1-propanoylpiperidin-4-yl)methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(2-methylacryloyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(2-methylpropanoyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(3,3-dimethylbutanoyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(cyclopentylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(cyclopropylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(ethylsulfonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(furan-3-ylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(methylsulfonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(phenylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(piperidin-1-ylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(pyrrolidin-1-ylcarbonyl)piperidin-4-yl]methyl]pyridine-2-sulfonamide”
“N-[6-cyano-1-[(1-methyl-1H-imidazol-5-yl)methyl]-1,2,3,4,6,7-hexahydroquinolin-3-yl]-N-[[1-(thiophen-3-ylcarbonyl)piperidin-4-yl]methyl]pyridine-2-sulfonamide”
“N-[6-cyano-1-[(1-methyl-1H-imidazol-5-yl)methyl]-1,2,3,4-tetrahydroquinolin-3-yl]-1-methyl-N-(1-methylethenyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(2-methylbenzyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(2-phenylethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(2-piperidin-1-ylethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(2-thiophen-3-ylethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(piperidin-4-ylmethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(pyridin-2-ylmethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(pyridin-3-ylmethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(quinolin-7-ylmethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-(thiophen-3-ylmethyl)-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[(5-phenyl-1,3-oxazol-4-yl)methyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[2-(1H-pyrrol-1-yl)ethyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[2-(trifluoromethyl)benzyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[2-[2-(trifluoromethyl)phenyl]ethyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[4-(1H-1,2,4-triazol-1-yl)benzyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[4-(1H-pyrazol-1-yl)benzyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[4-(1H-pyrrol-1-yl)benzyl]-1H-imidazole-4-sulfonamide”
“N-[6-cyano-1-[(1-methyl-1H-imidazol-5-yl)methyl]-1,2,3,4-tetrahydroquinolin-3-yl]-1-methyl-N-[4-(methylsulfonyl)benzyl]-1H-pyrazole-4-sulfonamide”
“N-[6-cyano-1-[(1-methyl-1H-imidazol-5-yl)methyl]-1,2,3,4-tetrahydroquinolin-3-yl]-1-methyl-N-[[5-(trifluoromethyl)furan-2-yl]methyl]-1H-imidazole-4-sulfonamide”
“N-[6-cyano-1-[(1-methyl-1H-imidazol-5-yl)methyl]-1,2,3,4-tetrahydroquinolin-3-yl]-3-fluoro-N-[4-(methylsulfonyl)benzyl]benzenesulfonamide”
“N-[6-cyano-1-[(1-methyl-1H-imidazol-5-yl)methyl]-1,2,3,4-tetrahydroquinolin-3-yl]-4-methoxy-N-[4-(methylsulfonyl)benzyl]benzenesulfonamide”
“N-[6-cyano-1-[(1-methyl-1H-imidazol-5-yl)methyl]-1,2,3,4-tetrahydroquinolin-3-yl]-N-(2-ethoxybenzyl)-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-(2-fluorobenzyl)-1-methyl-1H-imidazole-4-carboxamide”
“N-[6-cyano-1-[(1-methyl-1H-imidazol-5-yl)methyl]-1,2,3,4-tetrahydroquinolin-3-yl]-N-(2-fluorobenzyl)-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-(2-fluorobenzyl)-2,1,3-benzothiadiazole-4-sulfonamide”
“N-[6-cyano-1-[(1-methyl-1H-imidazol-5-yl)methyl]-1,2,3,4-tetrahydroquinolin-3-yl]-N-(2-fluorobenzyl)-2-methoxyacetamide”
“N-[6-cyano-1-[(1-methyl-1H-imidazol-5-yl)methyl]-1,2,3,4-tetrahydroquinolin-3-yl]-N-(2-fluorobenzyl)-3-(methylsulfanyl)propanamide”
“N-[6-cyano-1-[(1-methyl-1H-imidazol-5-yl)methyl]-1,2,3,4-tetrahydroquinolin-3-yl]-N-(2-fluorobenzyl)-N′,N′-dimethylsulfamide”
“N-[6-cyano-1-[(1-methyl-1H-imidazol-5-yl)methyl]-1,2,3,4-tetrahydroquinolin-3-yl]-N-(2-fluorobenzyl)acetamide”
“N-[6-cyano-1-[(1-methyl-1H-imidazol-5-yl)methyl]-1,2,3,4-tetrahydroquinolin-3-yl]-N-(2-fluorobenzyl)benzenesulfonamide”
“N-[6-cyano-1-[(1-methyl-1H-imidazol-5-yl)methyl]-1,2,3,4-tetrahydroquinolin-3-yl]-N-(2-fluorobenzyl)cyclopropanecarboxamide”
“N-[6-cyano-1-[(1-methyl-1H-imidazol-5-yl)methyl]-1,2,3,4-tetrahydroquinolin-3-yl]-N-(2-fluorobenzyl)ethanesulfonamide”
“N-[6-cyano-1-[(1-methyl-1H-imidazol-5-yl)methyl]-1,2,3,4-tetrahydroquinolin-3-yl]-N-(2-fluorobenzyl)methanesulfonamide”
“N-[6-cyano-1-[(1-methyl-1H-imidazol-5-yl)methyl]-1,2,3,4-tetrahydroquinolin-3-yl]-N-(2-fluorobenzyl)propane-1-sulfonamide”
“N-[6-cyano-1-[(1-methyl-1H-imidazol-5-yl)methyl]-1,2,3,4-tetrahydroquinolin-3-yl]-N-(2-fluorobenzyl)pyridine-2-sulfonamide”
“N-[6-cyano-1-[(1-methyl-1H-imidazol-5-yl)methyl]-1,2,3,4-tetrahydroquinolin-3-yl]-N-(2-methylbenzyl)pyridine-2-sulfonamide”
“N-[6-cyano-1-[(1-methyl-1H-imidazol-5-yl)methyl]-1,2,3,4-tetrahydroquinolin-3-yl]-N-(3-methoxypropyl)pyridine-2-sulfonamide”
“N-[6-cyano-1-[(1-methyl-1H-imidazol-5-yl)methyl]-1,2,3,4-tetrahydroquinolin-3-yl]-N-(5-methoxypentyl)pyridine-2-sulfonamide”
“N-[6-cyano-1-[(1-methyl-1H-imidazol-5-yl)methyl]-1,2,3,4-tetrahydroquinolin-3-yl]-N-(pyridin-2-ylmethyl)pyridine-2-sulfonamide”
“N-[6-cyano-1-[(1-methyl-1H-imidazol-5-yl)methyl]-1,2,3,4-tetrahydroquinolin-3-yl]-N-(pyridin-2-ylsulfonyl)glycine”
“N-[6-cyano-1-[(1-methyl-1H-imidazol-5-yl)methyl]-1,2,3,4-tetrahydroquinolin-3-yl]-N-(pyridin-3-ylmethyl)pyridine-2-sulfonamide”
“N-[6-cyano-1-[(1-methyl-1H-imidazol-5-yl)methyl]-1,2,3,4-tetrahydroquinolin-3-yl]-N-(quinolin-8-ylmethyl)pyridine-2-sulfonamide”
“N-[6-cyano-1-[(1-methyl-1H-imidazol-5-yl)methyl]-1,2,3,4-tetrahydroquinolin-3-yl]-N-[(1-methyl-1H-imidazol-4-yl)sulfonyl]glycine”
“N-[6-cyano-1-[(1-methyl-1H-imidazol-5-yl)methyl]-1,2,3,4-tetrahydroquinolin-3-yl]-N-[(2-phenyl-2H-1,2,3-triazol-4-yl)methyl]pyridine-2-sulfonamide”
“N-[6-cyano-1-[(1-methyl-1H-imidazol-5-yl)methyl]-1,2,3,4-tetrahydroquinolin-3-yl]-N-[(3,5-dimethylisoxazol-4-yl)methyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[(4-phenyl-1,3-oxazol-5-yl)methyl]pyridine-2-sulfonamide”
“N-[6-cyano-1-[(1-methyl-1H-imidazol-5-yl)methyl]-1,2,3,4-tetrahydroquinolin-3-yl]-N-[(5-methyl-3-phenylisoxazol-4-yl)methyl]pyridine-2-sulfonamide”
“N-[6-cyano-1-[(1-methyl-1H-imidazol-5-yl)methyl]-1,2,3,4-tetrahydroquinolin-3-yl]-N-[(5-methylisoxazol-3-yl)methyl]pyridine-2-sulfonamide”
“N-[6-cyano-1-[(1-methyl-1H-imidazol-5-yl)methyl]-1,2,3,4-tetrahydroquinolin-3-yl]-N-[2-(1,3-dioxo-1,3-dihydro-2H-isoindol-2-yl)ethyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[2-(1,3-dioxo-1,3-dihydro-2H-isoindol-2-yl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(1H-imidazol-1-yl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(1H-indol-3-yl)ethyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[2-(1H-pyrrol-1-yl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(2,3-dihydro-1,4-benzodioxin-5-yloxy)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(2,3-dihydro-1H-indol-1-yl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(2,5-dihydrothiophen-3-yl)ethyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[2-(2-fluorophenyl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(4-fluorophenyl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(5,5-dimethyl-1,3-dioxan-2-yl)ethyl]pyridine-2-sulfonamide”
“N-[6-cyano-1-[(1-methyl-1H-imidazol-5-yl)methyl]-1,2,3,4-tetrahydroquinolin-3-yl]-N-[2-(dimethylamino)ethyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[2-(trifluoromethyl)benzyl]pyridine-2-sulfonamide”
“N-[6-cyano-1-[(1-methyl-1H-imidazol-5-yl)methyl]-1,2,3,4-tetrahydroquinolin-3-yl]-N-[3-(1H-1,2,4-triazol-1-yl)benzyl]pyridine-2-sulfonamide”
“N-[6-cyano-1-[(1-methyl-1H-imidazol-5-yl)methyl]-1,2,3,4-tetrahydroquinolin-3-yl]-N-[3-(1H-pyrazol-1-yl)benzyl]pyridine-2-sulfonamide”
“N-[6-cyano-1-[(1-methyl-1H-imidazol-5-yl)methyl]-1,2,3,4-tetrahydroquinolin-3-yl]-N-[3-(1H-pyrrol-1-yl)benzyl]pyridine-2-sulfonamide”
“N-[6-cyano-1-[(1-methyl-1H-imidazol-5-yl)methyl]-1,2,3,4-tetrahydroquinolin-3-yl]-N-[3-(dimethylamino)propyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[4-(1,3-dioxo-1,3-dihydro-2H-isoindol-2-yl)butyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[4-(1,3-dioxo-1,3-dihydro-2H-isoindol-2-yl)butyl]pyridine-2-sulfonamide”
“N-[6-cyano-1-[(1-methyl-1H-imidazol-5-yl)methyl]-1,2,3,4-tetrahydroquinolin-3-yl]-N-[4-(3,4-dimethyl-2,6-dioxo-3,6-dihydropyrimidin-1 (2H)-yl)butyl]pyridine-2-sulfonamide”
“N-[6-cyano-1-[(1-methyl-1H-imidazol-5-yl)methyl]-1,2,3,4-tetrahydroquinolin-3-yl]-N-[4-[(1,3-dioxo-1,3-dihydro-2H-isoindol-2-yl)oxy]butyl]-1-methyl-1H-imidazole-4-sulfonamide”
“N-[6-cyano-1-[(1-methyl-1H-imidazol-5-yl)methyl]-1,2,3,4-tetrahydroquinolin-3-yl]-N-[4-[(1,3-dioxo-1,3-dihydro-2H-isoindol-2-yl)oxy]butyl]pyridine-2-sulfonamide”
“N-[6-cyano-1-[(1-methyl-1H-imidazol-5-yl)methyl]-1,2,3,4-tetrahydroquinolin-3-yl]-N-[[5-(trifluoromethyl)furan-2-yl]methyl]pyridine-2-sulfonamide”
“N-[6-cyano-1-[(1-methyl-1H-imidazol-5-yl)methyl]-2-oxo-1,2,3,4-tetrahydroquinolin-3-yl]-1-methyl-1H-imidazole-4-sulfonamide”
“N-[6-cyano-1-[(1-methyl-1H-imidazol-5-yl)methyl]-2-oxo-1,2,3,4-tetrahydroquinolin-3-yl]-1-methyl-1H-imidazole-4-sulfonamide”
“N-[6-cyano-1-[(1-methyl-1H-imidazol-5-yl)methyl]-2-oxo-1,2,3,4-tetrahydroquinolin-3-yl]-1-methyl-N-[4-(methylsulfonyl)benzyl]-1H-imidazole-4-sulfonamide”
“N-[6-cyano-1-[(1-methyl-1H-imidazol-5-yl)methyl]-2-oxo-1,2,3,4-tetrahydroquinolin-3-yl]-N-[4-(methylsulfonyl)benzyl]pyridine-2-sulfonamide”
“N-[[2-(2,3-dicarboxypropyl)-1-methyl-1H-imidazol-5-yl]methyl]-L-valyl-L-isoleucyl-L-alanine”
“N-[[2-(2,3-dicarboxypropyl)-1-methyl-1H-imidazol-5-yl]methyl]-L-valyl-L-phenylalanyl-L-methionine”
“N-[[2-(3,4-dicarboxybutyl)-1-methyl-1H-imidazol-5-yl]methyl]-L-valyl-L-isoleucyl-L-alanine”
“N-[[2-(3,4-dicarboxybutyl)-1-methyl-1H-imidazol-5-yl]methyl]-L-valyl-L-phenylalanyl-L-methionine”
“N-[[2-(4,5-dicarboxypentyl)-1-methyl-1H-imidazol-5-yl]methyl]-L-valyl-L-phenylalanyl-L-methionine”
“N-[[2-(4,5-dicarboxypentyl)-1-methyl-1H-imidazol-5-yl]methyl]-L-valyl-Lisoleucyl-L-alanine”
“N-{6-cyano-1-[(1-methyl-1H-imidazol-5-yl)methyl]-1,2,3,4-tetrahydroquinolin-3-yl}-N-(3-methoxypropyl)-1-methyl-1H-imidazole-4-sulfonamide”
“N2-[1-(2-aminoethyl)-6-cyano-1,2,3,4-tetrahydroquinolin-3-yl]-N-tert-butyl-N2-[(1-methyl-1H-imidazol-2-yl)sulfonyl]glycinamide”
“N2-[1-[2-(acetylamino)-4-methyl-1,3-thiazol-5-yl]-6-cyano-1,2,3,4-tetrahydroquinolin-3-yl]-N-tert-butyl-N2-[(1-methyl-1H-imidazol-2-yl)sulfonyl]glycinamide”
“N2-[6-cyano-1-[(1-methyl-1H-imidazol-5-yl)methyl]-1,2,3,4-tetrahydroquinolin-3-yl]-N2-(pyridin-2-ylsulfonyl)glycinamide”
Na-[(3S)-3-([[1-(4-cyanobenzyl)-1H-imidazol-5-yl]methyl]amino)-4-phenylbutanoyl]-L-phenylalaninamide
nanaomycin A
nanaomycin D
NH2-KTKCVFM
O-methylthysanone
oreganic acid
penicillic acid
pepticinnamin A
pepticinnamin B
pepticinnamin C
pepticinnamin D
pepticinnamin E
pepticinnamin F
Phenylglyoxal
preussomerin D
preussomerin G
“pyridine-2-sulfonic acid (1-benzenesulfonyl-piperidin-4-ylmethyl)-[6-cyano-1-(3-methyl-3H-imidazol-4-ylmethyl)-1,2,3,4-tetrahydro-quinolin-3-yl]-amide”
“pyridine-2-sulfonic acid [6-cyano-1-(3-methyl-3H-imidazol-4-ylmethyl)-1,2,3,4-tetrahydro-quinolin-3-yl]-(1-methanesulfonyl-piperidin-4-ylmethyl)-amide”
“pyridine-2-sulfonic acid [6-cyano-1-(3-methyl-3H-imidazol-4-ylmethyl)-1,2,3,4-tetrahydro-quinolin-3-yl]-(2-pyrazol-1-ylethyl)-amide”
“pyridine-2-sulfonic acid [6-cyano-1-(3-methyl-3H-imidazol-4-ylmethyl)-1,2,3,4-tetrahydro-quinolin-3-yl]-(4-fluoro-benzyl)-amide”
“pyridine-2-sulfonic acid [6-cyano-1-(3-methyl-3H-imidazol-4-ylmethyl)-1,2,3,4-tetrahydro-quinolin-3-yl]-(4-methanesulfonyl-benzyl)-amide”
“pyridine-2-sulfonic acid [6-cyano-1-(3-methyl-3H-imidazol-4-ylmethyl)-1,2,3,4-tetrahydro-quinolin-3-yl]-piperidin-4-ylmethylamide”
“pyridine-2-sulfonic acid [6-cyano-1-(3-methyl-3H-imidazol-4-ylmethyl)-1,2,3,4-tetrahydro-quinolin-3-yl]-[1-(2,2-dimethylpropionyl)-piperidin-4-ylmethyl]-amide”
“pyridine-2-sulfonic acid [6-cyano-1-(3-methyl-3H-imidazol-4-ylmethyl)-1,2,3,4-tetrahydro-quinolin-3-yl]-[1-(3,3,3-trifluoropropionyl)-piperidin-4-ylmethyl]-amide”
“pyridine-2-sulfonic acid [6-cyano-1-(3-methyl-3H-imidazol-4-ylmethyl)-1,2,3,4-tetrahydro-quinolin-3-yl]-[2-(2-trifluoromethyl-phenyl)-ethyl]-amide”
R115777
RPR113228
rupatadine
SCH-37370
SCH-44342
SCH207278
SCH58450
SCH66336
sclerotiorin
shikonine
Sodium deoxycholate
spiculisporic acid
TAN-1813
“tert-butyl (2Z,4E,8E)-5,9,13-trimethyl-3-(1-methyl-1H-imidazol-2-yl)tetradeca-2,4,8,12-tetraenoate”
“tert-butyl 4-(3-bromo-8-chloro-6,11-dihydro-5H-benzo[5,6]cyclohepta[1,2-b]pyridin-11-yl)-2-[(pyridin-3-ylmethyl)carbamoyl]piperazine-1-carboxylate”
tert-butyl 4-(3-[(4-cyanophenyl)[(1-methyl-1H-imidazol-5-yl)methyl]amino]-2-[[(1-methyl-1H-imidazol-4-yl)sulfonyl]amino]propyl)piperidine-1-carboxylate
tert-butyl 4-(3-[(4-cyanophenyl)[(1-methyl-1H-imidazol-5-yl)methyl]amino]-2-[[(2-methylphenyl)sulfonyl]amino]propyl)piperidine-1-carboxylate
“tert-butyl 4-[([1-[(1-methyl-1H-imidazol-5-yl)methyl]-6-phenyl-1,2,3,4-tetrahydroquinolin-3-yl][(1-methyl-1H-imidazol-4-yl)sulfonyl]amino)methyl]piperidine-1-carboxylate”
“tert-butyl 4-[([6-cyano-1-[(1-methyl-1H-imidazol-5-yl)methyl]-1,2,3,4-tetrahydroquinolin-3-yl][(1-methyl-1H-imidazol-4-yl)sulfonyl]amino)methyl]piperidine-1-carboxylate”
“tert-butyl 4-[[[6-cyano-1-[(1-methyl-1H-imidazol-5-yl)methyl]-1,2,3,4-tetrahydroquinolin-3-yl](pyridin-2-ylsulfonyl)amino]methyl]piperidine-1-carboxylate”
“tert-butyl N-[6-cyano-1-[(1-methyl-1H-imidazol-5-yl)methyl]-1,2,3,4-tetrahydroquinolin-3-yl]-N-(phenylcarbonyl)glycinate”
“tert-butyl N-[6-cyano-1-[(1-methyl-1H-imidazol-5-yl)methyl]-1,2,3,4-tetrahydroquinolin-3-yl]-N-[(1-methyl-1H-imidazol-4-yl)sulfonyl]glycinate”
“tert-butyl N-[6-cyano-1-[(1-methyl-1H-imidazol-5-yl)methyl]-1,2,3,4-tetrahydroquinolin-3-yl]-N-[(5-methylisoxazol-3-yl)carbonyl]glycinate”
“tert-butyl [2-([1-[(1-methyl-1H-imidazol-5-yl)methyl]-6-phenyl-1,2,3,4-tetrahydroquinolin-3-yl][(1-methyl-1H-imidazol-4-yl)sulfonyl]amino)ethyl]carbamate”
“tert-butyl [2-([6-cyano-1-[(1-methyl-1H-imidazol-5-yl)methyl]-1,2,3,4-tetrahydroquinolin-3-yl][(1-methyl-1H-imidazol-4-yl)sulfonyl]amino)ethyl]carbamate”
“tert-butyl [2-[[6-cyano-1-[(1-methyl-1H-imidazol-5-yl)methyl]-1,2,3,4-tetrahydroquinolin-3-yl](pyridin-2-ylsulfonyl)amino]ethyl]carbamate”
“tert-butyl [3-[[6-cyano-1-[(1-methyl-1H-imidazol-5-yl)methyl]-1,2,3,4-tetrahydroquinolin-3-yl](pyridin-2-ylsulfonyl)amino]propyl]carbamate”
“tert-butyl [3-[[6-cyano-1-[(1-methyl-1H-imidazol-5-yl)methyl]-1,2,3,4-tetrahydroquinolin-3-yl](pyridin-2-ylsulfonyl)amino]propyl]methylcarbamate”
“tert-butyl [4-[[6-cyano-1-[(1-methyl-1H-imidazol-5-yl)methyl]-1,2,3,4-tetrahydroquinolin-3-yl](pyridin-2-ylsulfonyl)amino]butyl]carbamate”
“tert-butyl [4-[[6-cyano-1-[(1-methyl-1H-imidazol-5-yl)methyl]-1,2,3,4-tetrahydroquinolin-3-yl](pyridin-2-ylsulfonyl)amino]butyl]ethylcarbamate”
“tert-butyl [4-[[6-cyano-1-[(1-methyl-1H-imidazol-5-yl)methyl]-1,2,3,4-tetrahydroquinolin-3-yl](pyridin-2-ylsulfonyl)amino]butyl]methylcarbamate”
tetrahydroquinoline
thysanone
tipifarnib
ubenimex
UCF116-A
UCF116-B
UCF76-A
UCF76-B
UCF76-C
valinoctin A
valinoctin B
WS5995-C
zaragozic acid A
“[(1-methyl-1H-imidazole-4-sulfonyl)-[1-(3-methyl-3H-imidazol-4-ylmethyl)-6-phenyl-1,2,3,4-tetrahydro-quinolin-3-yl]-amino]-acetic acid methyl ester”
“[(2E,6E)-3,7,11-trimethyldodeca-2,6,10-trien-1-ylidene]propanedioic acid”
“[(2E,6E)-3,7,11-trimethyldodeca-2,6,10-trien-1-yl]propanedioic acid”
“[(4E,8E)-5,9,13-trimethyltetradeca-4,8,12-trienoyl]sulfamic acid”
“[2-(4-[[[6-cyano-1-(3-methyl-3H-imidazol-4-ylmethyl)-1,2,3,4-tetrahydro-quinolin-3-yl]-(pyridine-2-sulfonyl)-amino]-methyl]-piperidin-1-yl)-2-oxo-ethyl]-carbamic acid tert-butyl ester”
“[3-(1-methyl-1H-imidazol-2-yl)propyl][(2E,6E)-3,7,11-trimethyldodeca-2,6,10-trien-1-yl]propanedioic acid”
[3-(4-chlorophenyl)-4-cyano-5-[(1-methylethyl)sulfanyl]thiophen-2-yl]methyl acetate
“[3-[[6-cyano-1-(3-methyl-3H-imidazol-4-ylmethyl)-1,2,3,4-tetrahydro-quinolin-3-yl]-(pyridine-2-sulfonyl)-amino]-propyl]-methyl-carbamic acid methyl ester”
“[[(2E,4E,8E)-5,9,13-trimethyltetradeca-2,4,8,12-tetraenoyl]amino]propanedioic acid”
“[[(2E,6E)-3,7,11-trimethyldodeca-2,6,10-trien-1-yl]oxy]propanedioic acid”
“[[(4E,8E)-5,9,13-trimethyltetradeca-4,8,12-trienoyl]amino]propanedioic acid”
“[[1-(3-benzyl-3H-imidazol-4-ylmethyl)-6-cyano-1,2,3,4-tetrahydro-quinolin-3-yl]-(1-methyl-1H-imidazole-4-sulfonyl)-amino]-acetic acid tert-butyl ester”
Miconazole

Another aspect of this disclosure includes the method of any preceding claim, further comprising administering an inhibitor of protein geranylgeranyl transferase (also known as GGTase). Specific GGTase inhibitors include:

EC 2.5.1.59 Geranylgeranyl Transferase Inhibitors
“(2R,3R,4S,5R)-2-(3,4-dichlorophenyl)-4-(hexylsulfanyl)-1-[(4-methylphenyl)sulfonyl]-5-propylpyrrolidine-3-carboxylic acid”
“(2R,3R,4S,5R)-2-(4-bromophenyl)-1-[(4-methylphenyl)sulfonyl]-4-(pentylsulfanyl)-5-propylpyrrolidine-3-carboxylic acid”
“(2R,3R,4S,5R)-2-(4-bromophenyl)-4-(hexylsulfanyl)-1-[(4-methylphenyl)sulfonyl]-5-propylpyrrolidine-3-carboxylic acid”
“(2R,3R,4S,5R)-2-(4-bromophenyl)-4-[(4-methoxyphenyl)sulfanyl]-1-[(4-methylphenyl)sulfonyl]-5-propylpyrrolidine-3-carboxylic acid”
“(2R,3R,4S,5R)-2-(4-bromophenyl)-5-ethyl-4-(hexylsulfanyl)-1-[(4-methylphenyl)sulfonyl]pyrrolidine-3-carboxylic acid”
“(2R,3R,4S,5R)-2-(4-bromophenyl)-5-hexyl-4-(hexylsulfanyl)-1-[(4-methylphenyl)sulfonyl]pyrrolidine-3-carboxylic acid”
“(2R,3R,4S,5R)-4-[(3-tert-butoxy-3-oxopropyl)sulfanyl]-2-(3-chlorophenyl)-5-(cyclopentylmethyl)-1-[(4-methylphenyl)sulfonyl]pyrrolidine-3-carboxylic acid”
“(2R,3R,4S,5R)-4-[(3-tert-butoxy-3-oxopropyl)sulfanyl]-2-(4-chlorophenyl)-1-[(4-chlorophenyl)sulfonyl]-5-(cyclopentylmethyl)pyrrolidine-3-carboxylic acid”
“(2R,3R,4S,5R)-4-[(3-tert-butoxy-3-oxopropyl)sulfanyl]-2-(4-chlorophenyl)-5-(cyclopentylmethyl)-1-(phenylsulfonyl)pyrrolidine-3-carboxylic acid”
“(2S,5R)-5-ethyl-2-(4-fluorophenyl)-1-tosyl-2,5-dihydro-1H-pyrrole-3-carboxylic acid”
“(2S,5S)-5-tert-butyl-2-(4-chlorophenyl)-1-[(2-methylphenyl)sulfonyl]-2,5-dihydro-1H-pyrrole-3-carboxylic acid”
“(2S,5S)-5-tert-butyl-2-(4-chlorophenyl)-1-[(2-methylphenyl)sulfonyl]-2,5-dihydro-1H-pyrrole-3-carboxylic acid”
“(2S,6S)-2,6-bis(4-chlorophenyl)-1-[(2-methylphenyl)sulfonyl]-1,2,5,6-tetrahydropyridine-3-carboxylic acid”
“(2S,6S)-6-(4-fluorophenyl)-1-[(4-methylphenyl)sulfonyl]-2-phenyl-1,2,5,6-tetrahydropyridine-3-carboxylic acid”
“(S)—N-(4-(3,4-dichlorophenoxy)benzyl)-6-(1H-indol-3-yl)piperazine-2,5-dione”
“(S)—N-(4-(3-chlorophenoxy)benzyl)-6-(1H-indol-3-yl)piperazine-2,5-dione”
“1-phosphono-(E,E,E)-geranylgeraniol”
“1-{2-[3-(1H-imidazol-4-ylmethyl)-2,4-dioxo-3,4-dihydro-2H-quinazolin-1-yl]-acetylamino}-cyclohexanecarboxylic acid”
“1-{2-[3-(1H-imidazol-4-ylmethyl)-2,4-dioxo-3,4-dihydro-2H-quinazolin-1-yl]-acetylamino}-cyclohexanecarboxylic acid methyl ester”
11-aminoundecylcarbonyl-L-cysteinyl-L-valyl-L-isoleucyl-L-leucine
2-aryl-4-aminobenzoic acid
“2-[3-(1H-imidazol-4-ylmethyl)-2,4-dioxo-3,4-dihydro-2H-quinazolin-1-yl]-N-(3-methyl-butyl)-acetamide”
“2-{2-[3-(1H-imidazol-4-ylmethyl)-2,4-dioxo-3,4-dihydro-2H-quinazolin-1-yl]acetylamino}-4-methyl-pentanoic acid methyl ester”
“2-{2-[3-(1H-imidazol-4-ylmethyl)-2,4-dioxo-3,4-dihydro-2H-quinazolin-1-yl]-acetylamino}-3-phenyl-propionic acid”
“2-{2-[3-(1H-imidazol-4-ylmethyl)-2,4-dioxo-3,4-dihydro-2H-quinazolin-1-yl]-acetylamino}-3-phenyl-propionic acid methyl ester”
“2-{2-[3-(1H-imidazol-4-ylmethyl)-2,4-dioxo-3,4-dihydro-2H-quinazolin-1-yl]-acetylamino}-4-methyl-pentanoic acid”
“2-{2-[3-(1H-imidazol-4-ylmethyl)-2,4-dioxo-3,4-dihydro-2H-quinazolin-1-yl]-acetylamino}-4-methyl-pentanoic acid methyl ester”
“2-{2-[3-(1H-imidazol-4-ylmethyl)-2,4-dioxo-3,4-dihydro-2H-quinazolin-1-yl]-acetylamino}-4-methylsulfanyl-butyric acid”
“2-{2-[3-(1H-imidazol-4-ylmethyl)-2,4-dioxo-3,4-dihydro-2H-quinazolin-1-yl]-acetylamino}-4-methylsulfanyl-butyric acid methyl ester”
3-(4′-farnesyloxy-3′-methoxyphenyl)-2-trans propenoic acid
3-(4′-farnesyloxy-3′-OH-phenyl)-2-trans propenoic acid
3-(4′-geranyloxy-3′-methoxyphenyl)-2-trans propenoic acid
3-(4′-geranyloxy-3′-methoxyphenyl)-2-trans propenoic acid ethyl ester
3-(4′-geranyloxy-3′-OH-phenyl)-2-trans propenoic acid
3-(4′-geranyloxy-3′-OH-phenyl)-2-trans propenoic acid ethyl ester
3-(4′-isopentenyloxy-3′-OH-phenyl)-2-trans propenoic acid
3-aza-geranylgeranyl-diphosphate
3-chloro-N-[2-oxo-2-[2-[[1-phenyl-3-(4-propoxyphenyl)pyrazol-4-yl]methylidene]hydrazinyl]ethyl]benzamide
4-[[([5-[(4-ethylphenoxy)methyl]-4-(1-phenylethyl)-4H-pyrazol-3-yl]sulfanyl)acetyl]amino]benzamide
“4-[[2-[[5-(2-methoxyphenyl)-4-phenethyl-1,2,4-triazol-3-yl]sulfanyl]acetyl]amino]benzamide”
“4-[[2-[[5-[(4-ethylphenoxy)methyl]-4-(1-phenylethyl)-1,2,4-triazol-3-yl]sulfanyl]acetyl]amino]benzamide”
auraptene
boropinic acid
collinin
Cys-3-(aminomethyl)benzoic acid-Leu
Cys-Val-Phe-Leu
diethyl dicarbonate
GGTI-2151
GGTI-2154
GGTI-297
GGTI-298
GGTI-DU40
“L-778,123”
N-(12-ammoniododecanoyl)-D-cysteinyl-L-valyl-L-isoleucyl-L-leucine trifluoroacetate
“N-(12-[[(3-[[(3R)-3-ammonio-4-phenylbutyl]oxy]-4,5-bis[[(3S)-3-ammonio-4-phenylbutyl]oxy]phenyl)carbonyl]amino]dodecanoyl)-L-cysteinyl-L-valyl-L-isoleucyl-L-leucine tris(trifluoroacetate)”
“N-(2,5-dichlorophenyl)-N′-[[3-(4-methylphenyl)-1-phenylpyrazol-4-yl]methylideneamino]oxamide”
“N-(4-[[(3-[[(3R)-3-ammonio-4-phenylbutyl]oxy]-4,5-bis[[(3S)-3-ammonio-4-phenylbutyl]oxy]phenyl)carbonyl]amino]butanoyl)-L-cysteinyl-L-valyl-L-isoleucyl-L-leucine tris(trifluoroacetate)”
N-benzyl-2-[(2-chlorobenzyl)[[5-(4-methylphenyl)-2H-tetrazol-2-yl]acetyl]amino]butanamide
N-benzyl-2-[(2-chlorophenyl)methyl-[2-[5-(4-methylphenyl)tetrazol-2-yl]acetyl]amino]butanamide
“N-[(E)-1-(benzylcarbamoyl)-2-[5-(3,4-dichlorophenyl)furan-2-yl]ethenyl]-4-methylbenzamide”
“N-[12-([[3,4,5-tris(3-ammoniopropoxy)phenyl]carbonyl]amino)dodecanoyl]-L-cysteinyl-L-valyl-L-isoleucyl-L-leucine tris(trifluoroacetate)”
N-[2-(benzylamino)-2-oxoethyl]-2-[5-(4-chlorophenyl)tetrazol-2-yl]-N-(4-propan-2-ylphenyl)acetamide
“N-[3-(benzylamino)-1-[5-(3,4-dichlorophenyl)furan-2-yl]-3-oxoprop-1-en-2-yl]-4-methylbenzamide”
“N-[6-(3,4,5-tris(3-amino-1-propoxy)benzoylamino)-undecylcarbonyl]-L-cysteinyl-L-valyl-L-isoleucyl-L-leucine”
“N-[6-(3,4,5-tris(3-amino-4-phenyl-1-butoxy)benzoylamino)-hexylcarbonyl]-L-cysteinyl-L-valyl-L-isoleucyl-L-leucine trifluoroacetate”
“N-[6-(3,4,5-tris(3-amino-4-phenyl-1-butoxy)benzoylamino)-propylcarbonyl]-L-cysteinyl-L-valyl-L-isoleucyl-L-leucine”
“N-[6-(3,4,5-tris(3-amino-4-phenyl-1-butoxy)benzoylamino)-undecylcarbonyl]-L-cysteinyl-L-valyl-L-isoleucyl-L-leucine”
NPFREKKFFCAIL
PD-083176
Phenylglyoxal
Thr-Lys-Cys-Val-Ile-Leu
Thr-Lys-Cys-Val-Ile-Met
umbelliprenine
“[3-(1H-imidazol-4-ylmethyl)-2,4-dioxo-3,4-dihydro-2H-quinazolin-1-yl]-acetic acid benzyl ester”
“{2-[3-(1H-imidazol-4-ylmethyl)-2,4-dioxo-3,4-dihydro-2H-quinazolin-1-yl]-acetylamino}-acetic acid”
“{2-[3-(1H-imidazol-4-ylmethyl)-2,4-dioxo-3,4-dihydro-2H-quinazolin-1-yl]-acetylamino}-acetic acid methyl ester”
L-cysteinyl-L-valyl-L-isoleucyl-L-leucine

In another aspect, in a similar fashion to protein prenylation is a method of any preceding claim, further comprising administering an inhibitor of the farnesyl-diphosphate farnesyl transferase (also known as Squalene synthase or SQS) enzyme or enzymatic step that diverges from of the isoprenoid biosynthetic pathway. This would act to prevent the production of cholesterol and other sterols and isoprenoids that are important to cellular response to stress. Inhibition of this pathway can allow for apoptosis in stressed cells that play a role in pyrophosphate presentation to the immune system.

In particular, protein farnesyl transfer inhibitors include:

EC 2.5.1.21 Farnesyl-Diphosphate Farnesyltransferase or Squalene Synthase Inhibitors
“(1-[[(1R,5S)-7-chloro-5-(2-chlorophenyl)-1-(2-methylpropyl)-2-oxo-1,2,4,5-tetrahydro-3H-3-benzazepin-3-yl]acetyl]piperidin-4-yl)acetic acid”
“(1-[[(3R,5S)-1-[3-(acetyloxy)-2,2-dimethylpropyl]-7-chloro-5-(2,3-dimethoxyphenyl)-2-oxo-1,2,3,5-tetrahydro-4,1-benzoxazepin-3-yl]acetyl]piperidin-4-yl)acetic acid”
“(1-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]piperidin-4-yl)acetic acid”
“(1-[[(6S)-8-chloro-6-(2,3-dimethoxyphenyl)-1-(propan-2-yl)-6,10b-dihydro-1H,4H-[2]benzoxepino[4,5-c][1,2]oxazol-4-yl]acetyl]piperidin-4-yl)acetic acid”
“(1R,5S)-7-chloro-5-(2-methoxyphenyl)-1-(2-methylpropyl)-3-[2-oxo-2-(piperidin-1-yl)ethyl]-1,3,4,5-tetrahydro-2H-3-benzazepin-2-one”
“(2E)-3-[(1R,5S)-3-(carboxymethyl)-7-chloro-5-(2-chlorophenyl)-2-oxo-2,3,4,5-tetrahydro-1H-3-benzazepin-1-yl]-2-methylprop-2-enoic acid”
(3R)-3-[[3-(benzyloxy)phenyl]ethynyl]-1-azabicyclo[2.2.2]octan-3-ol
“(3S)-1-(3-((4R,6S)-8-chloro-6-(2,3-dimethoxyphenyl)-4H,6H-pyrrolo[1,2-a][4,1]benzoxazepin-4-yl)propanoyl)-3-piperidine carboxylic acid”
“(3S)-1-[2-[(4R,6S)-8-chloro-6-(2,3-dimethoxyphenyl)-4H,6H-pyrrolo[1,2-a][4,1]benzoxazepin-4-yl]acetyl]-3-piperidinecarboxylic acid”
“1,3-diallyl-2-[3-(isopropylamino)propoxy]-9H-carbazole”
“1-(2-[2-[(4R,6S)-8-chloro-6-(2,3-dimethoxyphenyl)-4H,6H-pyrrolo[1,2-a][4,1]benzoxazepin-4-yl]ethyl]-2H-1,2,3,4-tetrazol-5-yl)cyclopropanecarboxylic acid”
“1-(3-((4R,6S)-8-chloro-6-(2,3-dimethoxyphenyl)-4H,6H-pyrrolo[1,2-a][4,1]benzoxazepin-4-yl)propanoyl)-3-azetidine carboxylic acid”
1-allyl-2-(3-anilinopropoxy)-9H-carbazole
1-allyl-2-[3-(benzylamino)propoxy]-9H-carbazole
1-allyl-2-[3-(benzylamino)propoxy]-9H-carbazole hydrochloride
1-allyl-2-[3-(cyclohexylamino)propoxy]-9H-carbazole
1-allyl-2-[3-(cyclopropylamino)propoxy]-9H-carbazole
1-allyl-2-[3-(isobutylamino)propoxy]-9H-carbazole
1-allyl-2-[3-(isopropylamino)propoxy]-9H-carbazole
1-allyl-2-[3-(isopropylamino)propoxy]-9H-xanthen-9-one
“1-[2-[(4R,6S)-8-chloro-6-(2,3-dimethoxyphenyl)-4H,6H-pyrrolo[1,2-a][4,1]benzoxazepin-4-yl]acetyl]-4-piperidinecarboxylic acid”
“1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazole-4-carboxylic acid”
“1-[2-[(4R,6S)-8-chloro-6-(2,3-dimethoxyphenyl)-4H,6H-pyrrolo[1,2-a][4,1]benzoxazepin-4-yl]ethyl]-1H-pyrazole-4-carboxylic acid”
“1-[[(1R,5R)-7-chloro-5-(2,3-dimethoxyphenyl)-1-(2-methylpropyl)-2-oxo-1,2,4,5-tetrahydro-3H-3-benzazepin-3-yl]acetyl]piperidine-4-carboxylic acid”
“1-[[(1R,5S)-7-chloro-5-(2,3-dimethoxyphenyl)-1-(2-methylpropyl)-2-oxo-1,2,4,5-tetrahydro-3H-3-benzazepin-3-yl]acetyl]piperidine-4-carboxylic acid”
“1-[[(1R,5S)-7-chloro-5-(2-chlorophenyl)-1-(2-methylpropyl)-2-oxo-1,2,4,5-tetrahydro-3H-3-benzazepin-3-yl]acetyl]piperidine-4-carboxylic acid”
“1-[[(1R,5S)-7-chloro-5-(2-methoxyphenyl)-1-(2-methylpropyl)-2-oxo-1,2,4,5-tetrahydro-3H-3-benzazepin-3-yl]acetyl]piperidine-4-carboxylic acid”
“1-[[(1S,5S)-7-chloro-5-(2,3-dimethoxyphenyl)-1-(2-methylpropyl)-2-oxo-1,2,4,5-tetrahydro-3H-3-benzazepin-3-yl]acetyl]piperidine-4-carboxylic acid”
“1-[[(6S)-8-chloro-6-(2,3-dimethoxyphenyl)-1-(propan-2-yl)-6,10b-dihydro-1H,4H-[2]benzoxepino[4,5-c][1,2]oxazol-4-yl]acetyl]piperidine-4-carboxylic acid”
“2-(1-(3-[(4R,6S)-8-chloro-6-(2,3-dimethoxyphenyl)-4H,6H-pyrrolo[1,2-a][4,1]benzoxazepin-4-yl]propanoyl)-3-azetidinyl)acetic acid”
“2-(1-[2-[(4R,6S)-8-chloro-6-(2,3-dimethoxyphenyl)-4H,6H-pyrrolo[1,2-a][4,1]benzoxazepin-4-yl]acetyl]-4-piperidinyl)acetic acid”
“2-(1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazol-4-yl)acetic acid”
“2-(1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazol-5-yl)acetic acid”
“2-(1-[2-[(4R,6S)-8-chloro-6-(2,3-dimethoxyphenyl)-4H,6H-pyrrolo[1,2-a][4,1]benzoxazepin-4-yl]ethyl]-1H-pyrazol-3-yl)acetic acid”
“2-(1-[2-[(4R,6S)-8-chloro-6-(2,3-dimethoxyphenyl)-4H,6H-pyrrolo[1,2-a][4,1]benzoxazepin-4-yl]ethyl]-1H-pyrazol-4-yl)acetic acid”
“2-(1-[2-[(4R,6S)-8-chloro-6-(2,3-dimethoxyphenyl)-4H,6H-pyrrolo[1,2-a][4,1]benzoxazepin-4-yl]ethyl]-1H-pyrazol-5-yl)acetic acid”
“2-(1-[2-[(4S,6R)-8-chloro-6-(2,3-dimethoxyphenyl)-4H,6H-pyrrolo[1,2-a][4,1]benzoxazepin-4-yl]acetyl]-4-piperidinyl)acetic acid”
“2-(1-[2-[8-chloro-6-(2,3-dimethoxyphenyl)-4H,6H-pyrrolo[1,2-a][4,1]benzoxazepin-4-yl]acetyl]-4-piperidinyl)acetic acid”
“2-(1-[3-[(4R,6S)-8-chloro-6-(2,3-dimethoxyphenyl)-4H,6H-pyrrolo[1,2-a][4,1]benzoxazepin-4-yl]propanoyl]-4-piperidinyl)acetic acid”
“2-(2-[2-[(4R,6S)-8-chloro-6-(2,3-dimethoxyphenyl)-4H,6H-pyrrolo[1,2-a][4,1]benzoxazepin-4-yl)ethyl]-2H-1,2,3,4-tetrazol-5-yl]-2-methylpropanoic acid”
“2-(2-[2-[(4R,6S)-8-chloro-6-(2,3-dimethoxyphenyl)-4H,6H-pyrrolo[1,2-a][4,1]benzoxazepin-4-yl)ethyl]-2H-1,2,3,4-tetrazol-5-yl]acetic acid”
“2-(2-[2-[(4R,6S)-8-chloro-6-(2,3-dimethoxyphenyl)-4H,6H-pyrrolo[1,2-a][4,1]benzoxazepin-4-yl]ethyl]-1H-1,2,3,4-tetrazol-5-yl)acetic acid”
“2-(4-[2-[(4R,6S)-8-chloro-6-(2,3-dimethoxyphenyl)-4H,6H-pyrrolo[1,2-a][4,1]benzoxazepin-4-yl]acetyl]-1-piperazinyl)acetic acid”
“2-(4-[3-[(4R,6S)-8-chloro-6-(2,3-dimethoxyphenyl)-4H,6H-pyrrolo[1,2-a][4,1]benzoxazepin-4-yl]propanoyl]-2-oxo-1-piperazinyl)acetic acid”
“2-([2-[(4R,6S)-8-chloro-6-(2,3-dimethoxyphenyl)-4H,6H-pyrrolo[1,2-a][4,1]benzoxazepin-4-yl]acetyl]amino)acetic acid”
“2-[(1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazol-4-yl)methoxy]-2-ethylbutanoic acid”
“2-[(1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazol-4-yl)methoxy]-2-methylpropanoic acid”
“2-[(1-[2-[(4R,6S)-8-chloro-6-(2,3-dimethoxyphenyl)-4H,6H-pyrrolo[1,2-a][4,1]benzoxazepin-4-yl]ethyl]-1H-1,2,3-triazol-4-yl)methoxy]acetic acid”
“2-[(1-[3-[(4R,6S)-8-chloro-6-(2,3-dimethoxyphenyl)-4H,6H-pyrrolo[1,2-a][4,1]benzoxazepin-4-yl]propanoyl]-4-piperidinyl)methoxy]acetic acid”
“2-[(1-[3-[(4R,6S)-8-chloro-6-(2,3-dimethoxyphenyl)-4H,6H-pyrrolo[1,2-a][4,1]benzoxazepin-4-yl]propanoyl]-4-piperidinyl)oxy]acetic acid”
“2-[(2-[2-[(4R,6S)-8-chloro-6-(2,3-dimethoxyphenyl)-4H,6H-pyrrolo[1,2-a][4,1]benzoxazepin-4-yl]ethyl]-2H-1,2,3,4-tetrazol-5-yl)methoxy]-2-methylpropanoic acid”
“2-[(6S)-8-chloro-6-(2,3-dimethoxyphenyl)-1-(propan-2-yl)-6,10b-dihydro-1H,4H-[2]benzoxepino[4,5-c][1,2]oxazol-4-yl]-1-[(3R)-3-hydroxypyrrolidin-1-yl]ethanone”
“2-[1,8-dichloro-6-(2,3-dimethoxyphenyl)-4H,6H-pyrrolo[1,2-a][4,1]benzoxazepin-4-yl]acetic acid”
“2-[2-[(4R,6S)-8-chloro-6-(2,3-dimethoxyphenyl)-4H,6H-pyrrolo[1,2-a][4,1]benzoxazepin-4-yl]ethyl]-2H-1,2,3,4-tetrazol-5-carboxylic acid”
2-[3-(isopropylamino)propoxy]-1-ethyl-9H-carbazole
2-[3-(isopropylamino)propoxy]-9H-carbazole
“2-[8-chloro-6-(1-naphthyl)-4H,6H-pyrrolo[1,2-a][4,1]benzoxazepin-4-yl]acetic acid”
“2-[8-chloro-6-(2,3-dichlorobenzoyl)-4H,6H-pyrrolo[1,2-a][4,1]benzoxazepin-4-yl]acetic acid”
“2-[8-chloro-6-(2,3-dihydro-1,4-benzodioxin-5-yl)-4H,6H-pyrrolo[1,2-a][4,1]benzoxazepin-4-yl]acetic acid”
“2-[8-chloro-6-(2,3-dimethoxyphenyl)-1-(4-morpholinylmethyl)-4H,6H-pyrrolo[1,2-a][4,1]benzoxazepin-4-yl]acetic acid”
“2-[8-chloro-6-(2,3-dimethoxyphenyl)-1-[(dimethylamino)methyl]-4H,6H-pyrrolo[1,2-a][4,1]benzoxazepin-4-yl]acetic acid”
“2-[8-chloro-6-(2,3-dimethoxyphenyl)-4H,6H-pyrrolo[1,2-a][4,1]benzoxazepin-4-yl]acetic acid”
2-[[(2Z)-2-(1-azabicyclo(2.2.2)oct-3-ylidene)-2-fluoroethyl]oxy]-9H-carbazole
“3-(2-[2-[(4R,6S)-8-chloro-6-(2,3-dimethoxyphenyl)-4H,6H-pyrrolo[1,2-a][4,1]benzoxazepin-4-yl]ethyl]-2H-1,2,3,4-tetrazol-5-yl)propanoic acid”
3-(4-quinolin-6-ylphenyl)quinuclidin-3-ol
“3-(biphenyl-4-yl)-2,3-dehydroquinuclidine”
3-(biphenyl-4-yl)-3-hydroxyquinuclidine
3-(biphenyl-4-yl)-4′[(t-butyldimethylsilyl)oxy]-3-hydroxyquinuclidine
“3-(biphenyl-4-yl-4′-hydroxy)-2,3-dehydroquinuclidine”
3-(biphenyl-4-ylmethyl)-1-azabicyclo[2.2.2]oct-2-ene
“3-([2-[(4R,6S)-8-chloro-6-(2,3-dimethoxyphenyl)-4H,6H-pyrrolo[1,2-a][4,1]benzoxazepin-4-yl]acetyl]amino)benzoic acid”
“3-([2-[(4R,6S)-8-chloro-6-(2,3-dimethoxyphenyl)-4H,6H-pyrrolo[1,2-a][4,1]benzoxazepin-4-yl]acetyl]amino)propionic acid”
“3-C-carboxy-2,4-dideoxy-2-dodec-11-en-1-ylpentaric acid”
“3-C-carboxy-2,4-dideoxy-2-dodecylpentaric acid”
“3-[(1-[3-[(4R,6S)-8-chloro-6-(2,3-dimethoxyphenyl)-4H,6H-pyrrolo[1,2-a][4,1]benzoxazepin-4-yl]propanoyl]-4-piperidinyl)oxy]propanoic acid”
“3-[(4R,6S)-8-chloro-6-(2,3-dimethoxyphenyl)-4H,6H-pyrrolo[1,2-a][4,1]benzoxazepin-4-yl]propionic acid”
3-[[4-(benzyloxy)phenyl]ethynyl]-1-azabicyclo[2.2.2]oct-2-ene
“4-[3-[(4R,6S)-8-chloro-6-(2,3-dimethoxyphenyl)-4H,6H-pyrrolo[1,2-a][4,1]benzoxazepin-4-yl]propanoyl]-2-morpholine carboxylic acid”
“6-([[(1R,5S)-7-chloro-5-(2-chlorophenyl)-1-(2-methylpropyl)-2-oxo-1,2,4,5-tetrahydro-3H-3-benzazepin-3-yl]acetyl]amino)hexanoic acid”
BMS 187745
BMS-188494
chlorogenic acid
CP-294838
CP-295697
deoxycholate
E5700
EP2302
EP2306
ER-27856
ER-28448
ER119884
ethyl 4-(1-azabicyclo[2.2.2]oct-2-en-3-yl)benzoate
“ethyl [(1R,5S)-7-chloro-5-(2-chlorophenyl)-1-(2-methylpropyl)-2-oxo-1,2,4,5-tetrahydro-3H-3-benzazepin-3-yl]acetate”
fenofibrate
Guanidinium chloride
lapaquistat
“methyl 2-[8-chloro-6-(2,3-dimethoxyphenyl)-4H,6Hpyrrolo[1,2-a][4,1]benzoxazepin-4-yl]acetate”
N-ethylmaleimide
N-isopropyl-biphenyloxypropylamine
“N-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]-L-aspartic acid”
RPR107393
squalestatin
T-91485
TAK-475
YM-53601
zaragozic acid
zaragozic acid A
“[(1R,5R)-7-chloro-1-(2-methylpropyl)-2-oxo-5-phenyl-1,2,4,5-tetrahydro-3H-3-benzazepin-3-yl]acetic acid”
“[(1R,5S)-1-benzyl-7-chloro-5-(2-chlorophenyl)-2-oxo-1,2,4,5-tetrahydro-3H-3-benzazepin-3-yl]acetic acid”
“[(1R,5S)-5-(2-bromophenyl)-7-chloro-1-(2-methylpropyl)-2-oxo-1,2,4,5-tetrahydro-3H-3-benzazepin-3-yl]acetic acid”
“[(1R,5S)-5-(2-chlorophenyl)-1-(2-methylpropyl)-2-oxo-1,2,4,5-tetrahydro-3H-3-benzazepin-3-yl]acetic acid”
“[(1R,5S)-5-(2-chlorophenyl)-7-fluoro-1-(2-methylpropyl)-2-oxo-1,2,4,5-tetrahydro-3H-3-benzazepin-3-yl]acetic acid”
“[(1R,5S)-5-(2-chlorophenyl)-7-methyl-1-(2-methylpropyl)-2-oxo-1,2,4,5-tetrahydro-3H-3-benzazepin-3-yl]acetic acid”
“[(1R,5S)-7-chloro-5-(2,3-dimethoxyphenyl)-1-(2-methylpropyl)-2-oxo-1,2,4,5-tetrahydro-3H-3-benzazepin-3-yl]acetic acid”
“[(1R,5S)-7-chloro-5-(2-chlorophenyl)-1-(2-hydroxy-2-methylpropyl)-2-oxo-1,2,4,5-tetrahydro-3H-3-benzazepin-3-yl]acetic acid”
“[(1R,5S)-7-chloro-5-(2-chlorophenyl)-1-(2-methylpropyl)-2-oxo-1,2,4,5-tetrahydro-3H-3-benzazepin-3-yl]acetic acid”
“[(1R,5S)-7-chloro-5-(2-methoxyphenyl)-1-(2-methylpropyl)-2-oxo-1,2,4,5-tetrahydro-3H-3-benzazepin-3-yl]acetic acid”
“[(4R,6R)-8-chloro-6-(2,3-dimethoxyphenyl)-1-(propan-2-yl)-6,10b-dihydro-1H,4H-[2]benzoxepino[4,5-c][1,2]oxazol-4-yl]acetic acid”
“[(6S)-8-chloro-6-(2,3-dimethoxyphenyl)-1-(propan-2-yl)-6,10b-dihydro-1H,4H-[2]benzoxepino[4,5-c][1,2]oxazol-4-yl]acetic acid”

Further aspects of this disclosure include a method of treating a disorder selected from the group consisting of an immune disorder, inflammatory disorder, or autoimmune disease, comprising administering to a patient in need thereof a therapeutically effective amount of a compound or combination of compounds of any preceding claim in order to ameliorate a symptom of the disorder.

In certain embodiments, the autoimmune disorder is ankylosing spondylitis, arthritis, rheumatoid arthritis, osteoarthritis, Chagas disease, dermatomyositis, diabetes mellitus type 1, endometriosis, Goodpasture's syndrome, Graves' disease, Guillain-Barré syndrome, Hashimoto's thyroiditis disease, Hidradenitis suppurativa, Kawasaki disease, IgA nephropathy, Idiopathic thrombocytopenic purpura, inflammatory bowel disease, Celiac's disease, Crohn's disease, eosinophilic gastroenteritis, ulcerative colitis, collagenous colitis, lymphocytic colitis, ischemic colitis, diversion colitis, Behcet's syndrome, infective colitis, indeterminate colitis interstitial cystitis, lupus, systemic lupus erythematosus, discoid lupus, drug-induced lupus, neonatal lupus, mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, temporal arteritis (also known as giant cell arteritis), vasculitis, vitiligo, Wegener's granulomatosis, alopecia areata, sarcoidosis, Addison's disease, or autoimmune hemolytic anemia.

Given the potential inability to accurately measure pyrophosphates in a subject, several other biomarkers that are normally associated with autoimmune disorders and diseases can be described. This include the use of IL-17, TNFα, RORγ, γδ lymphocytes, Vγ9Vδ2 lymphocytes, Vitamin D or other acceptable biomarkers that correlate with autoimmune disease or a particular autoimmune disease. Therefore additional aspects of this disclosure are a method of reducing (or increasing, in the case of Vitamin D) these markets in a subject with an autoimmune disease, comprising administering to a subject in need thereof an effective amount of a compound containing any one or more of compounds described in any preceding claims to reduce (increase for Vitamin D) the amount of any of these markers in a subject.

As used herein, the terms “subject” and “patient” are used interchangeable and refer to organisms to be treated by the methods of the present invention. Such organisms preferably include, but are not limited to, mammals and, most preferably, humans. Therefore another aspect is a method of any of the preceding claims wherein the subject is a human.

Aspects of this disclosure which describe methods of therapeutic delivery include a method of any preceding claim, wherein the agent is administered orally, intravenously, intramuscularly, subcutaneously, or transdermally. Additionally, the description of a method of any preceding claim, wherein any combination of two or more agents are taken simultaneously (concurrently) or at different times. An effective amount can be administered in one or more administrations, applications or dosages and is not intended to be limited to a particular formulation or administration route.

Another aspect of this disclosure is that one or more compounds of the disclosure may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H2O.

Additional embodiments include a compound derived from one or more compounds of any preceding claim and a pharmaceutically acceptable carrier. As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW, wherein W is C1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present disclosure compounded with a suitable cation such as Na+, NH4+, and NW4 (wherein W is a Ci-4 alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et ah, Camille G. (eds.) Handbook of Pharmaceutical Salts. Properties, Selection and Use. (2002) Zurich: Wiley-VCH; S. Berge et ah, Journal of Pharmaceutical Sciences (1977) 66(1) 1-19; P. Gould, International J. of Pharmaceutics (1986) 33 201-217; Anderson et ah, The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Additional embodiments of the invention include:
A. The use of a pharmaceutical formulation that inhibits the 2-C-methyl-D-erythritol 4-phosphate/1-deoxy-D-xylulose 5-phosphate pathway of isoprenoid biosynthesis to treat or to prevent autoimmune disease.
B. The use a pharmaceutical formulation of two or more compounds to treat or to prevent autoimmune disease where one compound inhibits the 3-hydroxy-3-methyl-glutaryl-CoA reductase enzyme in the mevalonate dependent pathway of isoprenoid biosynthesis (a statin belonging to the ATC class C10) and the other compound(s) are selected from the group consisting of: bisphosphonates (ATC class M05B), antibiotics (ATC classes J01 and J04), antiprotozoal (ATC class P01), immunosuppressants (ATC class L04), or corticosteroids for systemic use (ATC class H02).
C. The use of a pharmaceutical formulation of two or more compounds to treat or to prevent autoimmune disease where one compound inhibits farnesyl diphosphate synthases (a bisphosphonate belonging to the ATC class M05B) and the other compound(s) are selected from the group consisting of: statins (ATC class C10), antibiotics (ATC classes J01 and J04), antiprotozoal (ATC class P01), non-antibody immunosuppressants (ATC class L04), or corticosteroids for systemic use (ATC class H02).
D. The use of Embodiment A that includes a second or more pharmaceutical compound(s) belonging to the statin class of drugs (ATC class C10), bisphosphonates (ATC class M05B), antibiotics (ATC classes J01 and J04), antiprotozoal (ATC class P01), immunosuppressants (ATC class L04), or corticosteroids for systemic use (ATC class H02).
E. The use of Embodiment A that inhibits the 1-deoxy-D-xylulose-5-phosphate reductoisomerase enzymatic step of the 2-C-methyl-D-erythritol 4-phosphate/1-deoxy-D-xylulose 5-phosphate pathway of isoprenoid biosynthesis.
F. The use of Embodiment E that is fosmidomycin or a derivative of fosmidomycin (also known as 3-(Formyl-hydroxy-amino)propylphosphonic acid).
G. Any of the foregoing embodiments, where the autoimmune disease of is ankylosing spondylitis, arthritis, rheumatoid arthritis, osteoarthritis, Chagas disease, dermatomyositis, diabetes mellitus type 1, endometriosis, Goodpasture's syndrome, Graves' disease, Guillain-Barré syndrome, Hashimoto's thyroiditis disease, Hidradenitis suppurativa, Kawasaki disease, IgA nephropathy, Idiopathic thrombocytopenic purpura, inflammatory bowel disease, Celiac's disease, Crohn's disease, eosinophilic gastroenteritis, ulcerative colitis, collagenous colitis, lymphocytic colitis, ischemic colitis, diversion colitis, Behcet's syndrome, infective colitis, indeterminate colitis interstitial cystitis, lupus, systemic lupus erythematosus, discoid lupus, drug-induced lupus, neonatal lupus, mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, temporal arteritis (also known as giant cell arteritis), vasculitis, vitiligo, Wegener's granulomatosis, alopecia areata, sarcoidosis, Addison's disease, or autoimmune hemolytic anemia.

As indicated above, compounds to be administered to a patient are desirably in the form of a pharmaceutical composition. Accordingly, the invention provides pharmaceutical compositions comprising a therapeutic agent formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or one of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### DEFINITIONS

To facilitate an understanding of the present invention, a number of terms and phrases are defined below.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. Cycloalkyl groups can be fused to other cycloalkyl, aryl, or heterocyclyl groups. In certain embodiments, the cycloalkyl group is not substituted, i.e., it is unsubstituted.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic ring systems having two or more carbocyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. In certain embodiments, the aromatic ring is substituted at one or more ring positions with halogen, alkyl, hydroxyl, or alkoxyl. In certain other embodiments, the aromatic ring is not substituted, i.e., it is unsubstituted.

The term “aralkyl” refers to an alkyl group substituted with an aryl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted.

The term “heteroaralkyl” refers to an alkyl group substituted with a heteroaryl group.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. In certain embodiments, the heterocyclcyl group is not substituted, i.e., it is unsubstituted.

The term “heterocycloalkyl” is art-recognized and refers to a saturated heterocyclyl group as defined above.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; Or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R, may be joined to form a 3- to 12-membered ring, such as a 3- to 10-membered ring or a 5- to 6-membered ring.

The term “alkanoyl” as used herein refers to a radical —O—CO-alkyl.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating an autoimmune disorder, comprising administering to a patient in need thereof a therapeutically effective amount of an isoprenoid pathway inhibitor to treat the autoimmune disorder.

2. The method of claim 1, wherein the isoprenoid pathway inhibitor is a methyl-erythritol phosphate pathway (MEP pathway) inhibitor.

3. The method of claim 1, wherein the isoprenoid pathway inhibitor is a mevalonate pathway inhibitor.

4. The method of claim 2, further comprising administering a mevalonate pathway inhibitor.

5. A method of reducing the amount of a pyrophosphate selected from Isopentenyl Pyrophosphate (IPP), (E)-4-Hydroxy-3-methyl-but-2-enyl pyrophosphate (HMBpp), or Farnesyl Pyrophosphate in a patient suffering from an autoimmune disorder, comprising administering to a patient in need thereof an effective amount of an agent that directly or indirectly reduces the amount of a pyrophosphate selected from Isopentenyl Pyrophosphate (IPP), (E)-4-Hydroxy-3-methyl-but-2-enyl pyrophosphate (HMBpp), or Farnesyl Pyrophosphate in the patient.

6. The method of claim 5, wherein the agent is an isoprenoid biosynthetic pathway inhibitor.

7. The method of claim 6, wherein the isoprenoid pathway inhibitor is a methyl-erythritol phosphate pathway (MEP pathway) inhibitor.

8. The method of claim 6, wherein the isoprenoid pathway inhibitor is a mevalonate pathway inhibitor.

9. The method of claim 7 or claim 8, further comprising administering a mevalonate pathway inhibitor.

10. The method of any preceding claim, wherein any MEP pathway inhibitor inhibits the 1-deoxy-D-xylulose 5-phosphate synthase enzyme or enzymatic step in the MEP pathway.

11. The method of any preceding claim, wherein any MEP pathway inhibitor inhibits the 1-deoxy-D-xylulose 5-phosphate reductase (also known as the DOXP reductase, Dxr, or IspC) enzyme or enzymatic step in the MEP pathway.

12. The method of any preceding claim, wherein any MEP pathway inhibitor inhibits the 2-C-methyl-D-erythritol 2,4-cyclodiphosphate synthase enzyme or enzymatic step in the in the MEP pathway.

13. The method of any preceding claim, wherein any MEP pathway inhibitor inhibits the farnesyl diphosphate synthase enzyme or enzymatic step in the in the MEP pathway.

14. The method of any preceding claim, wherein any MEP pathway inhibitor inhibits biosynthesis of (E)-4-Hydroxy-3-methyl-but-2-enyl pyrophosphate.

15. The method of any preceding claim, wherein any MEP pathway inhibitor inhibits biosynthesis of Isopentenyl pyrophosphate.

16. The method of any preceding claim, wherein any MEP pathway inhibitor inhibits biosynthesis of farnesyl pyrophosphate.

17. The method of any one of claims 1-9, wherein any MEP pathway inhibitor is fosmidomycin.

18. The method of any one of claims 1-9, wherein any MEP pathway inhibitor is a fosmidomycin derivative.

19. The method of any one of claims 1-9, wherein any MEP pathway inhibitor is a thiazolo (3,2-a) pyrimidine.

20. The method of any one of claims 1-9, wherein any MEP pathway inhibitor is a bisphosphonate.

21. The method of any one of claims 1-9, wherein any MEP pathway inhibitor is Fosmidomycin, FR900098, Etidronate, Clodronate, Tiludronate, Pamidronate, Neridronate, Olpadronate, Alendronate, Ibandronate, Risedronate, or Zoledronate.

22. The method of any one of claims 1-21, wherein any MVA pathway inhibitor inhibits the 3-hydroxy-3-methyl-glutaryl-CoA reductase (also known as HMG-CoA reductase or HMGCR) enzyme or enzymatic step in the MVA pathway.

23. The method of any one of claims 1-21, wherein any MVA pathway inhibitor inhibits the farnesyl diphosphate synthase (also known as FPPS or FDPS) enzyme or enzymatic step in the in the MVA pathway.

24. The method of any one of claims 1-21, wherein any MVA pathway inhibitor inhibits biosynthesis of Isopentenyl Pyrophosphate (IPP).

25. The method of any one of claims 1-21, wherein any MVA pathway inhibitor inhibits biosynthesis of Farnesyl Pyrophosphate.

26. The method of any one of claims 1-21, wherein any MVA pathway inhibitor is a statin.

27. The method of any one of claims 1-21, wherein any MVA pathway inhibitor is a bisphosphonate.

28. The method of any one of claims 1-21, wherein any MVA pathway inhibitor is Etidronate, Clodronate, Tiludronate, Pamidronate, Neridronate, Olpadronate, Alendronate, Ibandronate, Risedronate, Zoledronate, Atorvastatin, Cerivastatin, Fluvastatin, Lovastatin, Mevastatin, Pitavastatin, Pravastatin, Rosuvastatin or Simvastatin.

29. The method of any preceding claim, further comprising administering an anti-microbial agent.

30. The method of claim 29, wherein the anti-microbial agent is an antibacterial.

31. The method of claim 29, wherein the anti-microbial agent is an antifungal.

32. The method of claim 29, wherein the anti-microbial agent is an anti-mycobacterial.

33. The method of claim 29, wherein the anti-microbial agent is an anti-parasitic.

34. The method of claim 29, wherein the anti-microbial agent is an anti-protozoal.

35. The method of claim 29, wherein the anti-microbial agent is an anti-helmintic.

36. The method of claim 29, wherein the anti-microbial agent is a tetracycline.

37. The method of claim 29, wherein the anti-microbial agent is a sulfonamide.

38. The method of claim 29, wherein the anti-microbial agent is an anti-folate.

39. The method of claim 29, wherein the anti-microbial agent is a macrolide.

40. The method of claim 29, wherein the anti-microbial agent is a lincoamide.

41. The method of claim 29, wherein the anti-microbial agent is an artemisinin.

42. The method of claim 29, wherein the anti-microbial agent has efficacy against parasites.

43. The method of claim 29, wherein the anti-microbial agent has efficacy against gram negative bacteria.

44. The method of claim 29, wherein the anti-microbial agent is clarithromycin, azithromycin, clindamycin, lincomycin, rapamycin, atovaquone, proguanil, methotrexate, pyrimethamine, trimethoprim, artemisinin, artesunate, artemether, chloroquine, hydroxychloroquine, primequine, amodiaquine, mefloquine, tetracycline, doxycycline, or minocycline.

45. The method of any preceding claim, further comprising administering an anti-inflammatory agent.

46. The method of claim 45, wherein the anti-inflammatory agent is a corticosteroid.

47. The method of claim 45, wherein the anti-inflammatory agent is a purine synthesis inhibitor.

48. The method of claim 45, wherein the anti-inflammatory agent is a pyrimidine synthesis inhibitor.

49. The method of claim 45, wherein the anti-inflammatory agent is an anti-folate compound.

50. The method of claim 45, wherein the anti-inflammatory agent is an mTOR inhibitor.

51. The method of claim 45, wherein the anti-inflammatory agent has antagonistic efficacy against IL-17 or the IL-17 receptor.

52. The method of claim 45, wherein the anti-inflammatory agent has antagonistic efficacy against TNFα or the TNFα receptor

53. The method of claim 45, wherein the anti-inflammatory agent has antagonistic efficacy against IL-6 or the IL-6 receptor

54. The method of claim 45, wherein the anti-inflammatory agent has antagonistic efficacy against IL-23 or the IL-23 receptor

55. The method of claim 45, wherein the anti-inflammatory agent has antagonistic efficacy against the ICOS receptor or the CD-28 receptor

56. The method of claim 45, wherein the anti-inflammatory agent has agonist efficacy towards the CTLA-4 receptor.

57. The method of claim 45, wherein the anti-inflammatory agent has agonist efficacy towards the PD-1 receptor.

58. The method of claim 45, wherein the anti-inflammatory agent has agonist efficacy towards Sphingosine-1-phosphate or the S1P receptor.

59. The method of claim 45, wherein the anti-inflammatory agent is prednisone, mycophenolic acid, azathioprine, leflunomide, teriflunomide, methotrexate, rapamycin, adalimumab, afelimomab, certolizumab pegol, golimumab, infliximab, nerelimomab, abatacept, belatacept, etanercept, pegsunercept, aflibercept, alefacept, rilonacept or fingolimod.

60. The method of any preceding claim, further comprising administering an inhibitor of the protein farnesyl transferase (also known as FTase) enzyme or enzymatic step that diverges from of the isoprenoid biosynthetic pathway.

61. The method of claim 60, wherein the protein farnesyl transferase is Manumycin A, Lonafarnib, Tipifarnib, FTI-276, or FTI-277.

62. The method of any preceding claim, further comprising administering an inhibitor of the protein geranylgeranyl transferase (also known as GGTase) enzyme or enzymatic step that diverges from of the isoprenoid biosynthetic pathway.

63. The method of claim 62, wherein the protein geranylgeranyl transferase is GGTI-297 or GGTI-298.

64. The method of any preceding claim, further comprising administering an inhibitor of the farnesyl-diphosphate farnesyl transferase (also known as Squalene synthase or SQS) enzyme or enzymatic step that diverges from of the isoprenoid biosynthetic pathway.

65. The method of claim 64, wherein the farnesyl-diphosphate farnesyl transferase is zaragozic acid, TAK-475, RPR 107393.

66. A method of reducing the amount of IL-17 in a subject with an autoimmune disorder, comprising administering to a subject in need thereof an effective amount of an isoprenoid pathway inhibitor to reduce the amount of IL-17 in a subject.

67. A method of reducing the amount of TNFα in a subject with an autoimmune disorder, comprising administering to a subject in need thereof an effective amount of an isoprenoid pathway inhibitor to reduce the amount of TNFα in a subject.

68. A method of reducing the activity of RORγ defined lymphocytes in a subject with an autoimmune disorder, comprising administering to a subject in need thereof an effective amount an isoprenoid pathway inhibitor to reduce the amount of RORγ defined lymphocytes in a subject.

69. A method of reducing the activity of γδ lymphocytes in a subject with an autoimmune disorder, comprising administering to a subject in need thereof an effective amount of an isoprenoid pathway inhibitor to reduce the amount of γδ lymphocytes in a subject.

70. A method of reducing the activity of Vγ9Vδ2 lymphocytes in a subject with an autoimmune disorder, comprising administering to a subject in need thereof an effective amount of an isoprenoid pathway inhibitor to reduce the amount of Vγ9Vδ2 lymphocytes in a subject

71. A method of increasing the presence of Vitamin D in a subject with an autoimmune disorder, comprising administering to a subject in need thereof an effective amount of an isoprenoid pathway inhibitor to increase the amount of Vitamin D in a subject

72. The method of any one of claims 66-71, wherein the isoprenoid pathway inhibitor is a methyl-erythritol phosphate pathway (MEP pathway) inhibitor.

73. The method of any one of claims 66-71, wherein the isoprenoid pathway inhibitor is a mevalonate pathway inhibitor.

74. The method of claim 72 or claim 73, further comprising administering a mevalonate pathway inhibitor.

75. The method of any of the preceding claims wherein the subject is a human.

76. The method of any preceding claim, wherein the autoimmune disorder is ankylosing spondylitis, arthritis, rheumatoid arthritis, osteoarthritis, Chagas disease, dermatomyositis, diabetes mellitus type 1, endometriosis, Goodpasture's syndrome, Graves' disease, Guillain-Barré syndrome, Hashimoto's thyroiditis disease, Hidradenitis suppurativa, Kawasaki disease, IgA nephropathy, Idiopathic thrombocytopenic purpura, inflammatory bowel disease, Celiac's disease, Crohn's disease, eosinophilic gastroenteritis, ulcerative colitis, collagenous colitis, lymphocytic colitis, ischemic colitis, diversion colitis, Behcet's syndrome, infective colitis, indeterminate colitis interstitial cystitis, lupus, systemic lupus erythematosus, discoid lupus, drug-induced lupus, neonatal lupus, mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, temporal arteritis (also known as giant cell arteritis), vasculitis, vitiligo, Wegener's granulomatosis, alopecia areata, sarcoidosis, Addison's disease, or autoimmune hemolytic anemia.

77. The method of any preceding claim, wherein the autoimmune disorder is ankylosing spondylitis, arthritis, rheumatoid arthritis, osteoarthritis, Behcet's syndrome, multiple sclerosis, psoriasis, or psoriatic arthritis.
